(19) United States
(12) Patent Application Publication
(10) Pub. No.: US 2026/0060956 A1
(43) Pub. Date: Mar. 5, 2026

(54) TREATING PANCREATIC CANCER

(71) Applicant: Immuneering Corporation, Cambridge, MA (US)

(72) Inventors: Brett Matthew Hall, San Diego, CA (US); Peter John King, San Diego, CA (US); Praveen Nair, San Diego, CA (US)

(21) Appl. No.: 19/318,266

(22) Filed: Sep. 3, 2025

Related U.S. Application Data
(60) Provisional application No. 63/690,489, filed on Sep. 4, 2024.
(60) Provisional application No. 63/690,485, filed on Sep. 4, 2024.
(60) Provisional application No. 63/693,481, filed on Sep. 11, 2024.
(60) Provisional application No. 63/693,465, filed on Sep. 11, 2024.
(60) Provisional application No. 63/707,934, filed on Oct. 16, 2024.
(60) Provisional application No. 63/718,226, filed on Nov. 8, 2024.
(60) Provisional application No. 63/742,365, filed on Jan. 6, 2025.
(60) Provisional application No. 63/823,526, filed on Jun. 13, 2025.

(51) Int. Cl. A61K 31/37; A61K 31/282; A61K 31/337; A61K 31/4745; A61K 31/513; A61K 31/7068; A61P 35/00

(52) U.S. Cl. CPC A61K 31/37; A61K 31/282; A61K 31/337; A61K 31/4745; A61K 31/513; A61K 31/7068; A61P 35/00

(57) ABSTRACT

Methods of treating pancreatic cancer comprising the administration of a therapeutically effective amount of dual-MEK inhibitor or a pharmaceutically acceptable salt thereof, wherein the dual-MEK inhibitor is administered to the patient in certain dose amounts. Some methods relate to treating pancreatic ductal adenocarcinoma (PDAC). Some methods relate to treating PDAC with one or more additional chemotherapy agents in combination with the dual-MEK inhibitor.

### INCORPORATION BY REFERENCE TO ANY PRIORITY APPLICATIONS

This patent application claims the benefit of, and priority to, U.S. Provisional Patent Application No. 63/690,489, filed Sep. 4, 2024; U.S. Provisional Patent Application No. 63/693,481, filed Sep. 11, 2024; U.S. Provisional Patent Application No. 63/707,934, filed Oct. 16, 2024; U.S. Provisional Patent Application No. 63/718,226, filed Nov. 8, 2024; U.S. Provisional Patent Application No. 63/690,485, filed Sep. 4, 2024; U.S. Provisional Patent Application No. 63/693,465, filed Sep. 11, 2024 U.S. Provisional Patent Application No. 63/742,365, filed Jan. 6, 2025; and U.S. Provisional Patent Application No. 63/823,526, filed Jun. 13, 2025; each of which are incorporated by reference herein in their entirety.

### TECHNICAL FIELD

The present disclosure relates to the fields of chemistry and medicine. More particularly, the present disclosure relates to methods of treatment of certain forms of cancer with MEK inhibitor therapies.

### BACKGROUND

Pancreatic cancer has one of the highest mortality rates among all cancers. Pancreatic ductal adenocarcinoma (PDAC) is one of the most malignant cancers, and numerous therapeutic approaches have been explored based on drug response findings from pancreatic cancer cells tested in culture models. The majority of PDAC tumors are driven by KRAS mutation-driven activation of MAPK signaling, thereby highlighting MEK as an important candidate target for therapeutic intervention in PDAC patients.

The standard first-line treatment for treating pancreatic cancer is gemcitabine (e.g., GEMZAR®), which was approved by the Food and Drug Administration (“FDA”) in 1996. Albumin bound paclitaxel (e.g., Abraxane®) in combination with gemcitabine was found to be well tolerated in advanced pancreatic cancer in a Phase I/II study and showed evidence of antitumor activity. Chemotherapy with one or more of 5-fluorouracil (5-FU) and gemcitabine has been shown to prolong survival in pancreatic cancer. Combination therapies including folinic acid (leucovorin or levoleucovorin), 5-fluorouracil, and irinotecan (FOLFIRI), folinic acid, 5-fluorouracil, irinotecan and oxaliplatin (FOLFIRINOX), or, less commonly, a combination of folinic acid, 5-fluorouracil, and oxaliplatin (FOLFOX) are also used to treat some pancreatic cancers. Thus, there is an urgent need for improvements in, and effective alternatives to, current therapies for pancreatic cancer.

### SUMMARY OF THE DISCLOSURE

Provided herein are methods of treating pancreatic cancer comprising administering to a subject in need thereof with a once-daily dose of atebimetinib (Compound A), or a pharmaceutically acceptable salt thereof:

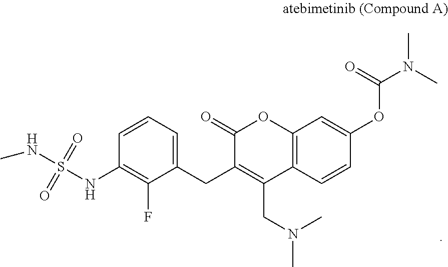

Atebimetinib (Compound A) is chemically defined with a molecular weight of 506.55 g/mol and a molecular formula of C₂₃H₂₇FN₄O₆S, and can be chemically named as 4-((dimethylamino)methyl)-3-(2-fluoro-3-((N-methylsulfamoyl)amino)benzyl)-2-oxo-2H-chromen-7-yl dimethylcarbamate. A method for preparing Compound A is provided in Example 1.

Methods of treating pancreatic cancer with atebimetinib disclosed herein provided a 94% overall survival (OS) at 6 months in first-line (1L) PDAC pancreatic cancer patients treated with 320 mg of atebimetinib administered po QD in combination with modified gemcitabine/nab-paclitaxel (mGnP) (i.e., 1,000 mg/m² gemcitabine+125 mg/m² nab-paclitaxel on days 1 & 15 of a treatment cycle, once every 4 weeks) (N=34). This is about 40% greater than the estimated benchmark 6-month OS (67%) for the standard of care treatment in this population (full dose and schedule gemcitabine/nab-paclitaxel treatment) based on the published results from the MPACT study (Pivotal Ph3 Study MPACT 2013 NEJM (PMID: 24131140) per 2024 JAMA Nichetti, et al. 7(1):e2350756).

In some embodiments, the cancer is pancreatic ductal adenocarcinoma (PDAC). In some embodiments, a method of treating pancreatic ductal adenocarcinoma (PDAC) comprises the step of administering 240 mg or 320 mg of Compound A once per day to a patient in need thereof. In some embodiments, a method of treating PDAC comprises orally administering (po) Compound A or a pharmaceutically acceptable salt thereof once per day (QD) to a patient in need thereof, in a dose providing a total of 240 mg of Compound A once per day (QD) to a patient in need thereof, as Compound A or a pharmaceutically acceptable salt thereof. In some embodiments, a method of treating PDAC comprises orally administering (po) Compound A or a pharmaceutically acceptable salt thereof once per day (QD) to a patient in need thereof, in a dose providing a total of 320 mg of Compound A once per day (QD) to a patient in need thereof, as Compound A or a pharmaceutically acceptable salt thereof.

In some embodiments, a method of treating pancreatic ductal adenocarcinoma (PDAC) comprises the step of administering 240 mg of Compound A once per day to a patient in need thereof as a monotherapy to treat the PDAC. In some embodiments, a method of treating pancreatic ductal adenocarcinoma (PDAC) comprises the step of administering 320 mg of Compound A once per day to a patient in need thereof as a monotherapy to treat the PDAC. In some embodiments, the method comprises administering 240 mg of Compound A once per day for 28 days in combination with: (a) one or more chemotherapy agents selected from the group consisting of gemcitabine, capecitabine, docetaxel and nab-paclitaxel, or (b) one or more chemotherapy agents selected from the group consisting of oxaliplatin, 5-fluorouracil and irinotecan. In some embodiments, the method comprises administering 320 mg of Compound A once per day for 28 days in combination with: (a) one or more chemotherapy agents selected from the group consisting of gemcitabine, capecitabine, docetaxel and nab-paclitaxel, or (b) one or more chemotherapy agents selected from the group consisting of oxaliplatin, 5-fluorouracil and irinotecan.

In some embodiments, a method of treating PDAC comprises orally administering (po) Compound A or a pharmaceutically acceptable salt thereof once per day (QD) to a patient in need thereof, in a dose providing a total of 240 mg of Compound A once per day (QD) to a patient in need thereof, as Compound A or a pharmaceutically acceptable salt thereof, as a monotherapy to treat the PDAC. In some embodiments, a method of treating PDAC comprises orally administering (po) Compound A or a pharmaceutically acceptable salt thereof once per day (QD) to a patient in need thereof, in a dose providing a total of 320 mg of Compound A once per day (QD) to a patient in need thereof, as Compound A or a pharmaceutically acceptable salt thereof, as a monotherapy to treat the PDAC.

In some embodiments, a method of treating pancreatic ductal adenocarcinoma (PDAC) comprises the steps of administering to a patient in need thereof: oral administration of 240 mg or 320 mg of Compound A once per day, in combination with a modified gemcitabine+nab-paclitaxel (mGnP) regimen where the mGnP regimen is administered on Day 1 and Day 15 of each 28-day cycle as follows: nab-paclitaxel 125 mg/m² via IV infusion over 30 min, followed immediately by gemcitabine 1000 mg/m² via IV infusion over 30 min.

In some embodiments, a method of treating pancreatic ductal adenocarcinoma (PDAC) comprises the steps of administering to a patient in need thereof: oral administration of 240 mg or 320 mg of Compound A once per day, in combination with a modified FOLFIRINOX (mFFX) regimen where the mFFX regimen is administered on Day 1 and Day 15 of each 28-day cycle for up to 6 cycles, as follows: oxaliplatin 85 mg/m² via IV infusion over 2 hours, folinic acid (leucovorin) 400 mg/m² via IV infusion over 2 hours, irinotecan 150 mg/m² via IV infusion over 90 minutes, and fluorouracil 2400 mg/m² continuous IV infusion given over 46 hours beginning Day 1 and ending on Day 3.

In some embodiments, the cancer optionally has a KRAS mutation such as a G12V, G12D, G12R, or G12H mutation.

In some embodiments, an oral dosage form of Compound A is provided. In some embodiments, an oral dosage form such as a tablet or capsule can contain Compound A or a pharmaceutically acceptable salt thereof as the API and one or more excipients such as a dry binder, tablet disintegrant, an absorbent, filler or diluent, a lubricant, anti-adherent, binder, thickening agent, viscosity-increasing agent, coating agent and film former. In some embodiments, an oral dosage form such as a tablet or capsule can contain 20 mg, 120 mg or 160 mg of Compound A or a pharmaceutically acceptable salt thereof as the API and one or more intra-granulation excipients such as a dry binder, tablet disintegrant, an absorbent, filler or diluent, a lubricant, anti-adherent, binder, thickening agent, viscosity-increasing agent, coating agent and film former and one or more extra granulation excipients such as additional Compound A API granulate and a lubricant or film forming agent. In some embodiments, an oral dosage form such as a tablet or capsule can contain 20 mg, 120 mg or 160 mg of Compound A or a pharmaceutically acceptable salt thereof as the API and one or more intra-granulation excipients such as microcrystalline cellulose, crospovidone Type A, and hydroxypropyl cellulose, and one or more extra-granulation excipients such as API granulate of Compound A and magnesium stearate. In some embodiments, an oral dosage form such as a tablet or capsule can contain about 20% w/w of Compound A or a pharmaceutically acceptable salt thereof as the API and about 60-70% w/w microcrystalline cellulose, about 5% w/w crospovidone Type A, and about 10% w/w hydroxypropyl cellulose, and one or more extra-granulation excipients such as API granulate of Compound A (95-99.5% w/w) and magnesium stearate (0.5-5% w/w).

### BRIEF DESCRIPTION OF THE DRAWINGS

FIGS. 1A and 1B are graphs summarizing results obtained from preclinical testing of Compound A in xenograft tumor models (tumor volume) for pancreatic cancer (KRAS-G12C and KRAS-G12V).

FIG. 2A are graphs showing data from the head-to-head comparison of Compound A+/−sotorasib (AMG-510) using a MIA PaCa-2 Xenograft Tumor Model (Tumor Volume).

FIG. 2B are graphs showing data from the head-to-head comparison of Compound A, sotorasib (AMG-510) and adagrasib using a CAPAN-2 Xenograft Tumor Model (Tumor Volume).

FIG. 3 is graph showing the mapping of biomarker sensitive patient populations (GENIE) for pancreatic cancer.

FIG. 4A is a graph showing PK/PD (pERK) for Compound A from patient 3 after receiving 160 mg Compound A administered orally (po) once daily (QD) (cycle 1, day 15).

FIG. 4B is a graph showing PK/PD (pMEK) for Compound A from patient 3 after receiving 160 mg Compound A administered orally (po) once daily (QD) (cycle 1, day 15).

FIG. 4C is a graph showing PK/PD (pERK) for Compound A from patient 4 after receiving 160 mg Compound A administered orally (po) once daily (QD) (cycle 1, day 15).

FIG. 4D is a graph showing PK/PD (pMEK) for Compound A from patient 4 after receiving 160 mg Compound A administered orally (po) once daily (QD) (cycle 1, day 15).

FIG. 5A is a graph showing PK/PD (pERK) for Compound A from patient 1 after receiving 40 mg Compound A administered orally (po) once daily (QD) (cycle 1, day 1).

FIG. 5B is a graph showing PK/PD (pMEK) for Compound A from patient 1 after receiving 40 mg Compound A administered orally (po) once daily (QD) (cycle 1, day 1).

FIG. 6A is a graph showing PK/PD (pERK) for Compound A from patients after receiving 80 mg Compound A administered orally (po) once daily (QD) (cycle 1, day 1).

FIG. 6B is a graph showing PK/PD (pMEK) for Compound A from patient 2 after receiving 80 mg Compound A administered orally (po) once daily (QD) (cycle 1, day 1).

FIG. 7A is a graph showing PK/PD (pERK) for Compound A from patient 5 after receiving 320 mg administered orally (po) once daily (QD) po (cycle 1, day 1).

FIG. 7B is a graph showing PK/PD (pMEK) for Compound A from patient 5 after receiving 320 mg administered orally (po) once daily (QD) po (cycle 1, day 1).

FIG. 7C is a graph showing PK/PD (pERK) for Compound A from patient 5 after receiving 320 mg administered orally (po) once daily (QD) po (cycle 1, day 15).

FIG. 7D is a graph showing PK/PD (pMEK) for Compound A from patient 5 after receiving 320 mg administered orally (po) once daily (QD) po (cycle 1, day 15).

FIG. 8A is a graph showing PK/PD (pERK) for Compound A from patient 6 after receiving 320 mg administered orally (po) once daily (QD) po (cycle 1, day 1).

FIG. 8B is a graph showing PK/PD (pMEK) for Compound A from patient 6 after receiving 320 mg administered orally (po) once daily (QD) po (cycle 1, day 1).

FIG. 8C is a graph showing PK/PD (pERK) for Compound A from patient 6 after receiving 320 mg administered orally (po) once daily (QD) po (cycle 1, day 15).

FIG. 8D is a graph showing PK/PD (pMEK) for Compound A from patient 6 after receiving 320 mg administered orally (po) once daily (QD) po (cycle 1, day 15).

FIG. 9A is a graph showing PK/PD (pERK) for Compound A from patient 7 after receiving 320 mg administered orally (po) once daily (QD) po (cycle 1, day 1).

FIG. 9B is a graph showing PK/PD (pMEK) for Compound A from patient 7 after receiving 320 mg administered orally (po) once daily (QD) po (cycle 1, day 1).

FIG. 9C is a graph showing PK/PD (pERK) for Compound A from patient 7 after receiving 320 mg administered orally (po) once daily (QD) po (cycle 1, day 15).

FIG. 9D is a graph showing PK/PD (pMEK) for Compound A from patient 7 after receiving 320 mg administered orally (po) once daily (QD) po (cycle 1, day 15).

FIG. 10A is a graph showing plasma concentration (PK) for Compound A from patient 13 after receiving 240 mg Compound A administered orally (po) once daily (QD) at cycle 1, day 1 and at cycle 1, day 15.

FIG. 10B is a graph showing PK/PD (pERK) for Compound A from patient 13 after receiving 240 mg Compound A administered orally (po) once daily (QD) at cycle 1, day 1 and at cycle 1, day 15.

FIG. 10C is a graph showing plasma concentration (PK) for Compound A from patient 16 after receiving 320 mg Compound A administered orally (po) once daily (QD) at cycle 1, day 1 and at cycle 1, day 15.

FIG. 10D is a graph showing PK/PD (pERK) for Compound A from patient 16 after receiving 320 mg Compound A administered orally (po) once daily (QD) at cycle 1, day 1 and at cycle 1, day 15.

FIG. 11A is a schematic diagram of an end-to-end translational modeling approach in support of Compound A.

FIG. 11B is a table providing certain Phase 1 clinical trial summary parameters for Compound A.

FIG. 12A is a graph showing 3D-TGA Compound A dose responses calculated for sensitive tumor models.

FIG. 12B is a graph showing 3D-TGA Compound A dose responses calculated for intermediate tumor models.

FIG. 12C is a graph showing 3D-TGA Compound A dose responses calculated for resistant tumor models.

FIG. 13 is a schematic depicting examples of various therapeutic applications of Compound A both as monotherapy and Compound A in combination with other therapies, for the treatment of certain RAS and RAF mutations.

FIG. 14A are dose response curves obtained in the humanized 3D Tumor Growth Assay (3D-TGA) with Compound A±gemcitabine with MIA PaCa-2 cells.

FIG. 14B are dose response curves obtained in the humanized 3D Tumor Growth Assay (3D-TGA) with Compound A±gemcitabine with Pane 03-27 (KRAS G12V) cells.

FIG. 14C are dose response curves obtained in the humanized 3D Tumor Growth Assay (3D-TGA) with Compound A±gemcitabine with Panc1 (KRAS-G12D) cells.

FIG. 15A are dose response curves obtained in the humanized 3D Tumor Growth Assay (3D-TGA) with Compound A±paclitaxel with MIA PaCa-2 cells.

FIG. 15B are dose response curves obtained in the humanized 3D Tumor Growth Assay (3D-TGA) with Compound A±paclitaxel with Panc 03-27 (KRAS G12V) cells.

FIG. 15C are dose response curves obtained in the humanized 3D Tumor Growth Assay (3D-TGA) with Compound A±paclitaxel with Panc1 (KRAS-G12D) cells.

FIG. 16 illustrates a distribution of RAS^{mut} across patients in the GENIE (v13.0) database.

FIG. 17A illustrates mutation profiles across pancreatic cancer indications in GENIE (v13) patient database.

FIG. 17B illustrates mutation profiles across colorectal cancer indications in GENIE (v13) patient database.

FIG. 18A illustrates a representative subset of compound A dose responses for sensitive (IC₅₀ <1 micromolar) cell lines shown in the table below the graph.

FIG. 18B illustrates a representative subset of compound A dose responses for intermediate (IC₅₀ ≥1 and >25% reduction at 10 micromolar) cell lines shown in the table below the graph.

FIG. 18C illustrates a representative subset of compound A dose responses for resistant (not susceptible or intermediate, and otherwise resistant in 3D-TGA response) cell lines shown in the table below the graph.

FIG. 19 illustrates a table of humanized 3D-TGA response based on tumor tissue and RAS mutation status.

FIG. 20 illustrates a table of KRAS G12 variant and Associated 3D-TGA response category.

FIG. 21A is a graph showing the RAS mutations reported at enrollment (N=41) for phase 1 human clinical trial patients treated with Compound A.

FIG. 21B is a graph showing cancer diagnosis for the patients reported at enrollment (N=41) for phase 1 human clinical trial patients treated with Compound A.

FIG. 22 is a graph showing pharmacokinetic (PK) and pharmacodynamic (PD) data obtained from patients treated with Compound A.

FIG. 23 is a graph of model-predicted response for patient profiles in pancreatic adenocarcinoma (PAAD) in GENIE 15.0.

FIG. 24A is a graph showing data obtained from an evaluation of Compound A (A), gemcitabine (GEM) or nab-paclitaxel (PAC) individually or in the specified combinations, as compared to vehicle using a MIA PaCa-2 KRAS G12C mutant xenograft tumor model in athymic nude mice (Tumor Volume).

FIG. 24B is a graph showing a magnified portion of the graph in FIG. 24A, showing data obtained from an evaluation of Compound A (A), gemcitabine (GEM) or nab-paclitaxel (PAC) individually or in the specified combinations, as compared to vehicle using a MIA PaCa-2 KRAS G12C mutant xenograft tumor model in athymic nude mice (Tumor Volume).

FIG. 25A is a graph showing data obtained from an evaluation of Compound A (A), and 5-fluorouracil (5-FU) individually or in combination, as compared to vehicle using a MIA PaCa-2 KRAS G12C mutant xenograft tumor model in athymic nude mice (Tumor Volume).

FIG. 25B is a graph showing a magnified portion of the graph in FIG. 25A, showing data obtained from an evaluation of Compound A (A), and 5-fluorouracil (5-FU) individually or in combination, as compared to vehicle using a MIA PaCa-2 KRAS G12C mutant xenograft tumor model in athymic nude mice (Tumor Volume).

FIG. 26A is a graph showing the results form an enrichment analysis of recurrently-mutated genes under continuous treatment in protein-protein interaction hubs for nab-paclitaxel, showing the number of mice per treatment group and a scatter plot of the corresponding odds ratio.

FIG. 26B is a graph showing the results form an enrichment analysis of recurrently-mutated genes under continuous treatment in protein-protein interaction hubs for gemcitabine, showing the number of mice per treatment group and a scatter plot of the corresponding odds ratio.

FIG. 26C is a graph showing the results form an enrichment analysis of recurrently-mutated genes under continuous treatment in protein-protein interaction hubs for 5-fluorouracil, showing the number of mice per treatment group and a scatter plot of the corresponding odds ratio.

FIG. 26D is a graph showing the results form an enrichment analysis of recurrently-mutated genes under continuous treatment in protein-protein interaction hubs for Compound A, showing the number of mice per treatment group and a scatter plot of the corresponding odds ratio.

FIG. 26E is a graph showing the results form an enrichment analysis of recurrently-mutated genes under continuous treatment in protein-protein interaction hubs for a combination of Compound A, gemcitabine and nab-paclitaxel, showing the number of mice per treatment group and a scatter plot of the corresponding odds ratio.

FIG. 26F is a graph showing the results form an enrichment analysis of recurrently-mutated genes under continuous treatment in protein-protein interaction hubs for a combination of Compound A, and 5-fluorouracil (5-FU), showing the number of mice per treatment group and a scatter plot of the corresponding odds ratio.

FIG. 27 is a graph showing 3D-TGA Compound A dose responses calculated for PaCaDD-137 cells.

FIG. 28A is a bar graph showing RECIST SLD (mm) for target lesions from a patient in a Phase 1 clinical trial treated with Compound A.

FIG. 28B is a graph showing CA-19-9 measurements from a patient in a Phase 1 clinical trial treated with Compound A.

FIG. 28C is a graph showing ctDNA measurements for KRAS G12V from a patient in a Phase 1 clinical trial treated with Compound A.

FIG. 29A is a bar graph showing RECIST SLD (mm) for target lesions from a patient in a Phase 1 clinical trial treated with Compound A.

FIG. 29B is a graph showing CA-19-9 measurements from a patient in a Phase 1 clinical trial treated with Compound A.

FIG. 29C is a graph showing ctDNA measurements for KRAS G12D and NRAS G13D from a patient in a Phase 1 clinical trial treated with Compound A.

FIG. 30A is a bar graph showing RECIST SLD (mm) for target lesions from a patient in a Phase 1 clinical trial treated with Compound A.

FIG. 30B is a graph showing CA-19-9 measurements from a patient in a Phase 1 clinical trial treated with Compound A.

FIG. 30C is a graph showing ctDNA measurements for KRAS G12D from a patient in a Phase 1 clinical trial treated with Compound A.

FIG. 31A is a bar graph showing RECIST SLD (mm) for target lesions from a patient in a Phase 1 clinical trial treated with Compound A.

FIG. 31B is a graph showing CA-19-9 measurements from a patient in a Phase 1 clinical trial treated with Compound A.

FIG. 31C is a graph showing ctDNA measurements for KRAS G12D from a patient in a Phase 1 clinical trial treated with Compound A.

FIG. 32 is a graph of the best percentage change in RECIST SLD for patients having pancreatic cancer harboring certain G12 or GNAS mutations treated with 240 mg QD or 320 mg QD of Compound A (po) in combination with modified gemcitabine/nab-paclitaxel in the Phase 2 clinical trial results disclosed herein.

FIG. 33A is a graph of the best percentage change in RECIST SLD for patients having pancreatic cancer harboring certain G12 mutations treated with 240 mg QD or 320 mg QD of Compound A (po) in combination with modified FOLFIRINOX in the Phase 2 clinical trial results disclosed herein.

FIG. 33B is a graph of the CA-19-9 levels from patients having pancreatic cancer harboring certain G12 mutations treated with 240 mg QD or 320 mg QD of Compound A (po) in combination with modified FOLFIRINOX in the Phase 2 clinical trial results disclosed herein.

FIG. 34 is a graph of the best percentage change in RECIST SLD for patients having pancreatic cancer harboring certain G12 mutations treated with 320 mg QD as a monotherapy in the Phase 2 clinical trial results disclosed herein.

FIG. 35A is a graph of overall survival (OS) probability by month in a human clinical trial of atebimetinib (320 mg QD) in the treatment of first line pancreatic (PDAC) patients, in combination with modified gemcitabine and nab-paclitaxel treatment, compared to third party published benchmark OS data obtained from different patients in the Phase 3 MPACT clinical trial.

FIG. 35B is a graph of the overall survival (OS) data from patients treated with atebimetinib in FIG. 35A compared to third party published benchmark OS data obtained from other published clinical trial data.

FIG. 36A is a graph showing the progression free survival probability by month in a human clinical trial of atebimetinib (320 mg QD) in the treatment of first line pancreatic (PDAC) patients, in combination with modified gemcitabine and nab-paclitaxel treatment.

FIG. 36B is a graph showing the progression free survival (PFS) probability by month in a human clinical trial of atebimetinib (320 mg QD) combined with modified gemcitabine and nab-paclitaxel treatment in the treatment of first line pancreatic (PDAC) patients, as shown in FIG. 36A, compared to third party published benchmark PFS probability data obtained from different patients in the Phase 3 MPACT clinical trial.

FIG. 36C is a graph of the progression free survival (PFS) data from patients treated with atebimetinib in FIG. 36A compared to third party published benchmark PFS probability data obtained from other published clinical trial data.

### DETAILED DESCRIPTION

In some embodiments, methods of therapeutically treating pancreatic cancer are provided, where the methods comprise administering a dual-MEK inhibitor to a subject in need thereof. In some aspects, the present disclosure provides methods for treating pancreatic ductal adenocarcinoma (PDAC) by administering a dual-MEK inhibitor characterized by a high maximum plasma concentration (Cmax) and a short plasma drug half-life. In some embodiments, the method includes sequential doses that are administered at a dose interval (i.e., time between doses) that is greater than the plasma drug half-life. In some embodiments, the dual-MEK inhibitor is Compound A, or a pharmaceutically acceptable salt thereof.

In some embodiments, the method of treating pancreatic cancer comprises administering Compound A,

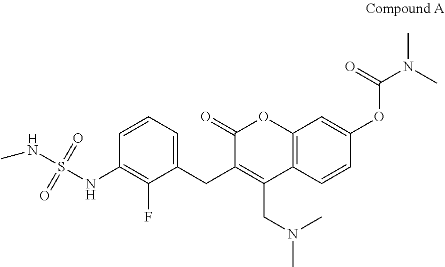

or a pharmaceutically acceptable salt thereof, once-daily to a patient in need thereof. In some embodiments, the patient is administered a dose of about 240 mg to about 320 mg of Compound A once-daily to the patient in need thereof.

Compound A is chemically defined with a molecular weight of 506.55 g/mol and a molecular formula of C₂₃H₂₇FN₄O₆S, and can be chemically named as 4-((dimethylamino)methyl)-3-(2-fluoro-3-((N-methylsulfamoyl)amino)benzyl)-2-oxo-2H-chromen-7-yl dimethylcarbamate. The method for synthesizing Compound A is detailed in Example 1.

Compound A is a dual MEK1/2 inhibitor useful to inhibit the activation of ERK while preventing RAF-mediated pathway reactivation. Compound A is a potent, non-ATP competitive selective dual MEK inhibitor with the biochemical inhibitory IC₅₀ for activated ERK of less than 100 nM across several cancer disease types, with the most sensitive being predominantly melanoma and pancreatic cancer cell lines that harbor activating MAPK pathway mutations. Corresponding reductions in pMEK were observed in cells treated with 100 nM Compound A, confirming that Compound A was not sensitive to the MAPK pathway feedback reactivation events, such as CRAF-bypass, that are a known limitation of the first generation MEK inhibitors currently in clinical use. Compound A has demonstrated potent inhibition of cell proliferation, and has been shown to have in vivo anti-tumor activity in mouse syngeneic and human xenograft models of activating MAPK pathway mutations in KRAS, NRAS, HRAS, BRAF, NF1, and KSR.

In primary pharmacodynamic assays, Compound A inhibited pERK across multiple tumor cells with half maximal inhibitory concentrations (IC₅₀) values ranging from 24.2 nM to 83.2 nM, with the most sensitive models being predominantly melanoma and pancreatic cancer cell lines that harbor activating MAPK pathway mutations in K isomer of rat sarcoma (KRAS), N isomer of rat sarcoma (NRAS), H isomer of rat sarcoma (HRAS), BRAF, neurofibromatosis type 1 (NF1), and/or kinase suppressor of Ras (KSR). Corresponding reductions in pMEK were observed with treatment of cells with 100 nM Compound A, confirming that Compound A was not sensitive to the MAPK pathway feedback reactivation events in RAS mutant models, such as CRAF-bypass.

Compound A resists MAPK pathway reactivation by reducing phosphorylation (activation) of MEK, enhancing its ability to target RAS mutant cells, where CRAF-bypass is particularly active. Compound A's unique pharmacokinetic profile allows it to disrupt the MAPK pathway in a deep but cyclic cadence. Deep, cyclic inhibition is a novel strategy for targeting MEK that is intended to take advantage of the fact that tumor cells with activating MAPK mutations are addicted to constant MAPK pathway signaling, while healthy cells are not. Deep, cyclic inhibition is designed to prevent tumor cells from sustaining proliferative signaling, while improving overall tolerability by reducing the impact on healthy cells relative to chronic ablation that is foundational in first and second generation MEK inhibitors. Compound A was designed to exploit tumor addiction to MAPK signaling while sparing healthy cells that utilize but are not addicted to the same pathway.

In some embodiments, methods of treating pancreatic cancer include treating PDAC with a RAS mutation. In some embodiments, the RAS mutation is KRAS-mutated PDAC. In some embodiments, the cancer is pancreatic cancer with a KRAS G12C mutation. In some embodiments, the cancer is pancreatic cancer with a G12D mutation. In some embodiments, the cancer is pancreatic cancer with a G12V mutation. Compound A has been evaluated in a wide range of human and murine solid tumor models, including those with activating MAPK pathway mutations in KRAS, NRAS, HRAS, BRAF, NF1, and KSR. Among others, Compound A was investigated in MIA PaCa-2 (KRAS^{G12C} PDAC), and Capan-2 (KRAS^{G12V} PDAC) mouse tumor models at various dose ranges. Significant tumor reduction was observed at all doses and in all models, lending evidence that Compound A activity is not dependent upon specific RAS mutations or RAS paralogues.

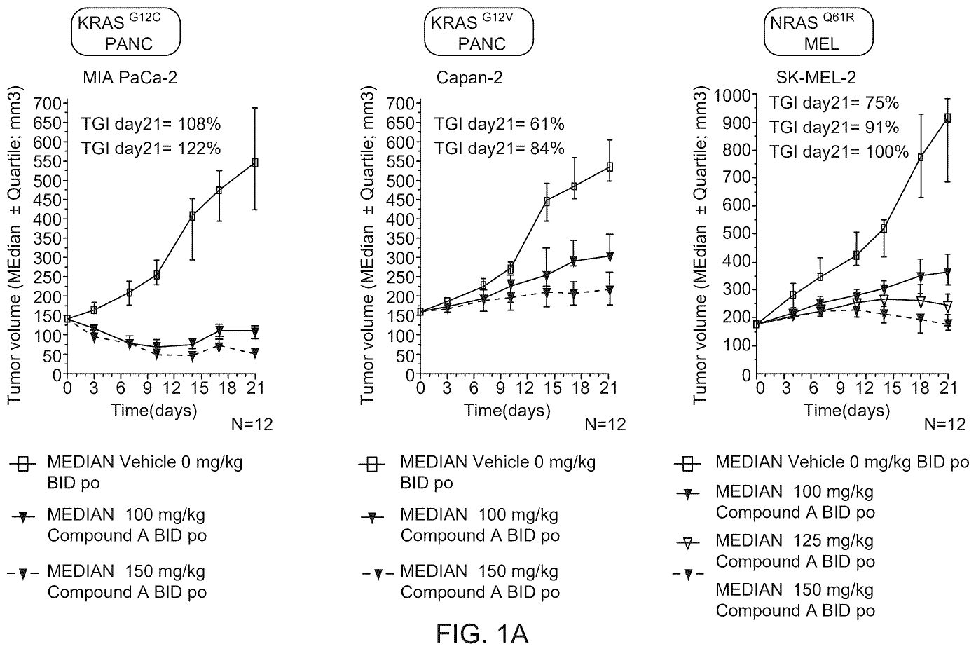

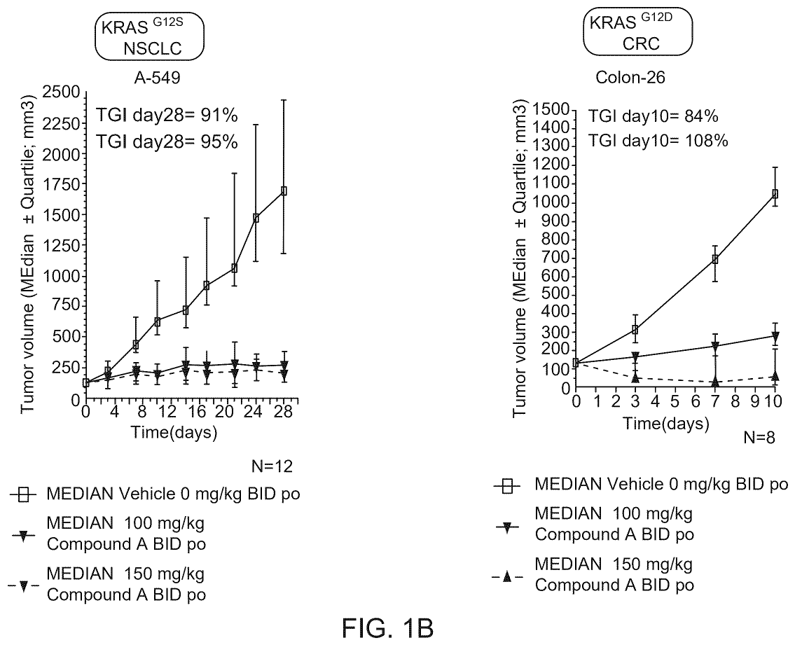

In head-to-head nonclinical studies, Compound A was compared to certain current FDA-approved MEK, BRAF and KRAS^{G12C} inhibitors in KRAS-, NRAS-, and BRAF-mutated solid tumor rodent models representing lung, colon, pancreas, and skin cancers; in each study, it demonstrated tumor stasis or regression while maintaining body weight. For instance, Compound A (100 and 150 mg/kg BID) was compared to the commercially available KRAS inhibitors sotorasib (30 and 100 mg/kg QD) and adagrasib (30 and 100 mg/kg QD) in the Capan-2 (KRAS^{G12D}) model, with Compound A showing efficacy in a non-KRAS^{G12C} model. In the KRAS^{G12C} MIA Paca-2 pancreatic model, similar tumor growth inhibition was observed between Compound A and sotorasib, and tumor regressions with improved durability of response (DoR) were demonstrated when Compound A and sotorasib were used in combination. FIGS. 1A and 1B are a summary of results obtained from preclinical testing of Compound A in various xenograft tumor models, for pancreatic cancer (KRAS-G12C and KRAS-G12V). Compound A was well tolerated with a median body weight loss ≤3-6% of baseline at top doses. Maximum effective dose range in mice (plasma t1/2=1.3 hours) is 100 mg/kg to 150 mg/kg BID po.

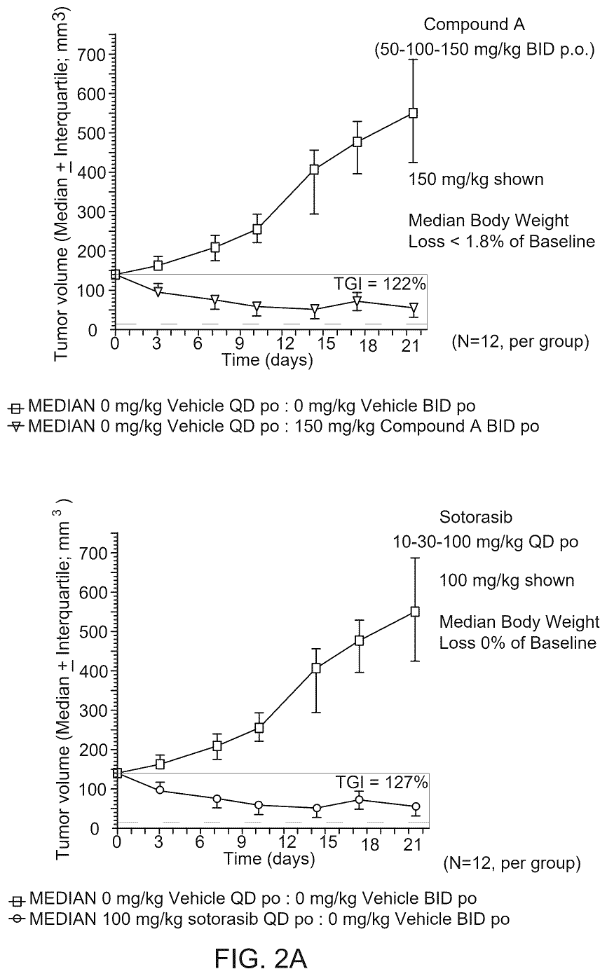

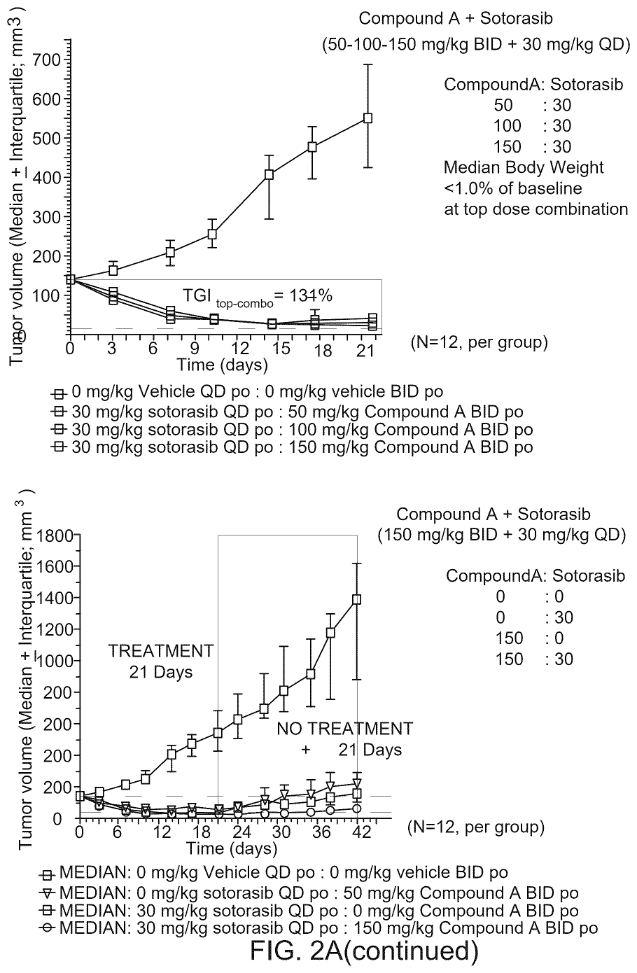

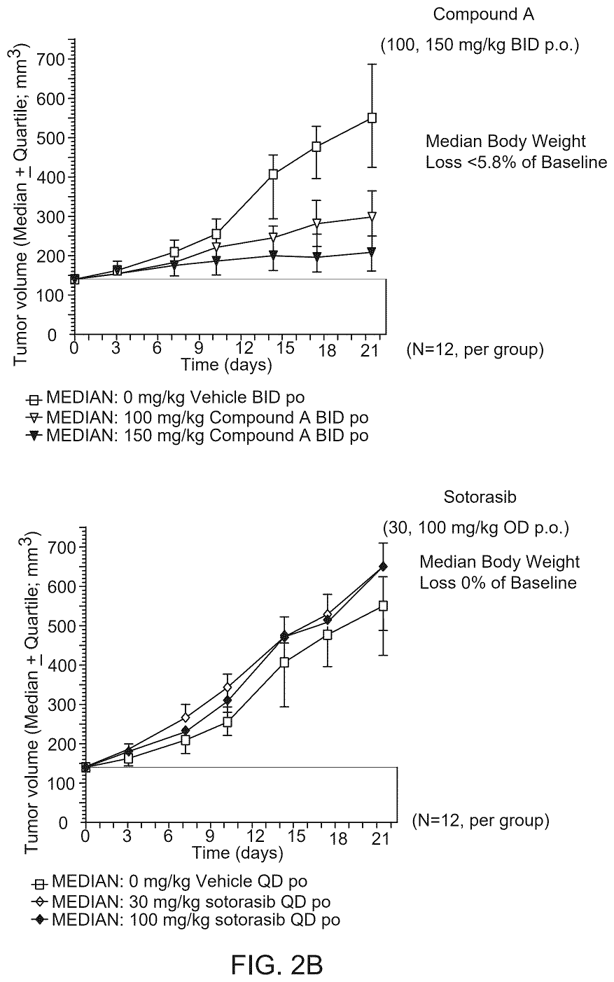

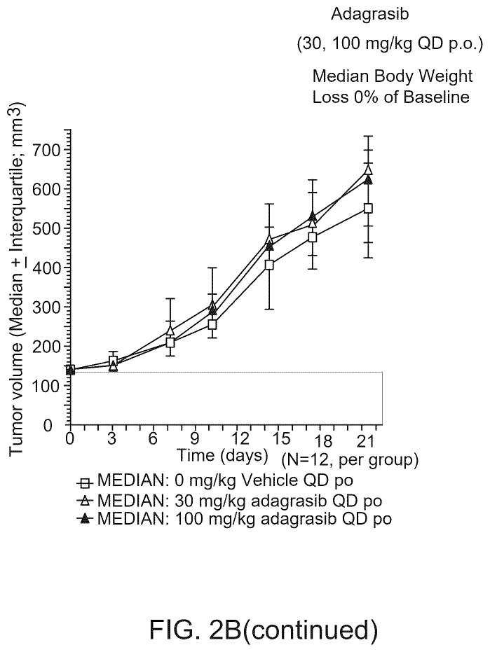

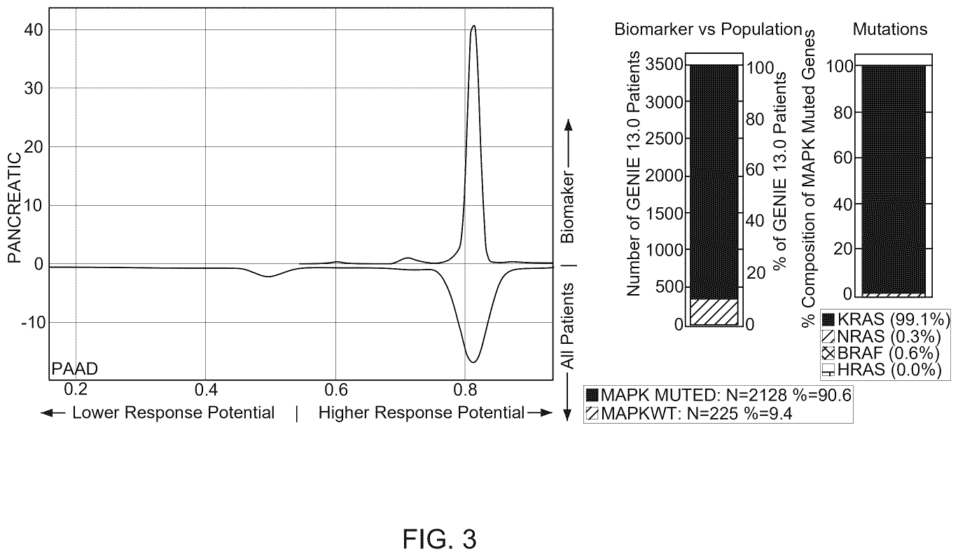

Compound A was evaluated head-to-head against sotorasib (AMG-510) and adagrasib alone for 21 days in the Capan-2 (KRAS G12V) Pancreatic Xenograft Tumor Model in Athymic Nude Mice. FIG. 2A shows data from the head-to-head Comparison of Compound A+/−sotorasib (AMG-510) using a MIA PaCa-2 Xenograft Tumor Model: Tumor Volume. As shown in FIG. 2B, Compound A demonstrated tumor regression as compared to sotorasib or adagrasib, with insignificant BWL. FIG. 3 is graph showing the mapping of biomarker sensitive patient populations (GENIE) for pancreatic cancer.

In some embodiments, Compound A or a pharmaceutically acceptable salt thereof is administered to the patient once daily in a dose amount providing 160-320 mg of Compound A to treat PDAC. In some embodiments, Compound A or a pharmaceutically acceptable salt thereof is administered to the patient once daily in a dose amount providing 160-320 mg of Compound A to treat KRAS mutated PDAC. In some embodiments, Compound A or a pharmaceutically acceptable salt thereof is administered to the patient once daily in a dose amount providing 160, 240 or 320 mg of Compound A to treat PDAC. In some embodiments, Compound A or a pharmaceutically acceptable salt thereof is administered to the patient once daily in a dose amount providing 160, 240 or 320 mg of Compound A to treat KRAS mutated PDAC. In some embodiments, Compound A or a pharmaceutically acceptable salt thereof is administered to the patient once daily in a dose amount providing 160 mg of Compound A to treat PDAC. In some embodiments, Compound A or a pharmaceutically acceptable salt thereof is administered to the patient once daily in a dose amount providing 240 mg of Compound A to treat KRAS mutated PDAC. In some embodiments, the dual-MEK inhibitor is Compound A or a pharmaceutically acceptable salt thereof is administered to the patient once daily in a dose amount providing 240 mg of Compound A to treat PDAC. In some embodiments, the dual-MEK inhibitor is Compound A or a pharmaceutically acceptable salt thereof is administered to the patient once daily in a dose amount providing 160 mg of Compound A to treat KRAS mutated PDAC. In some embodiments, Compound A or a pharmaceutically acceptable salt thereof is administered to the patient once daily in a dose amount providing 320 mg of Compound A to treat PDAC. In some embodiments, Compound A or a pharmaceutically acceptable salt thereof is administered to the patient once daily in a dose amount providing 320 mg of Compound A to treat KRAS mutated PDAC.

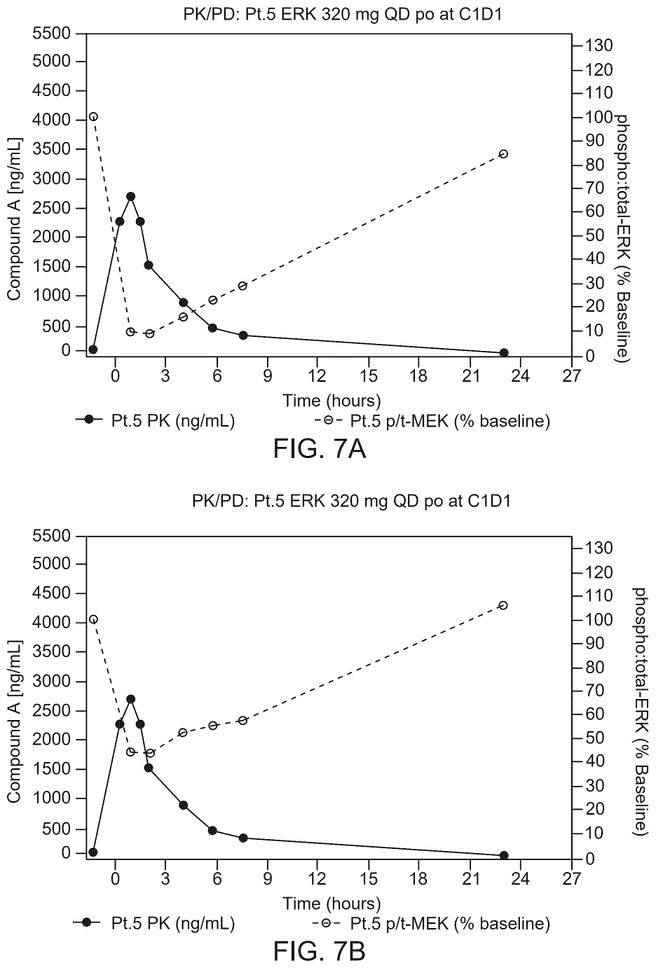

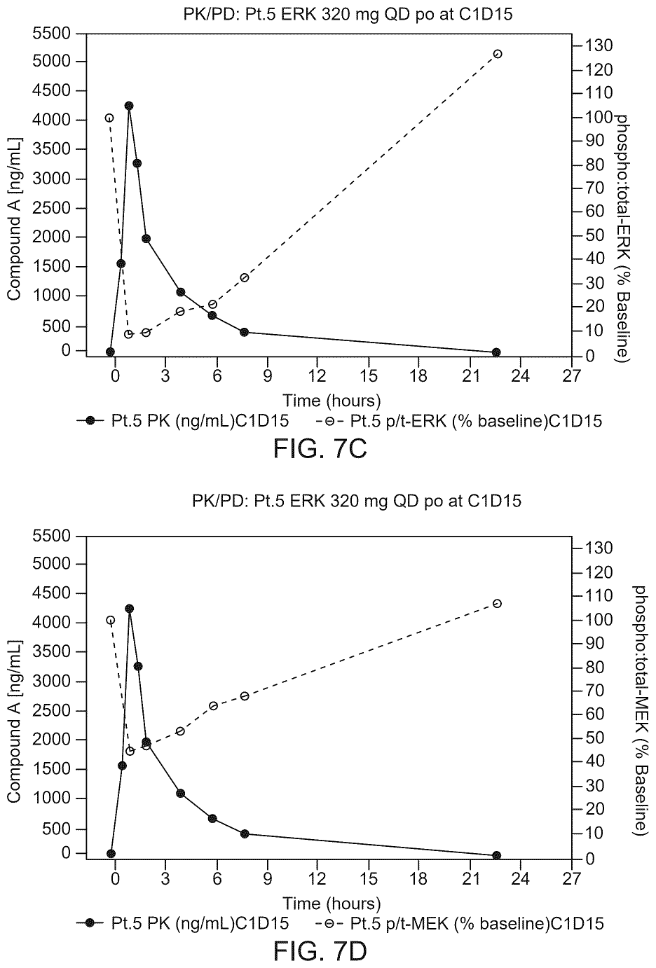

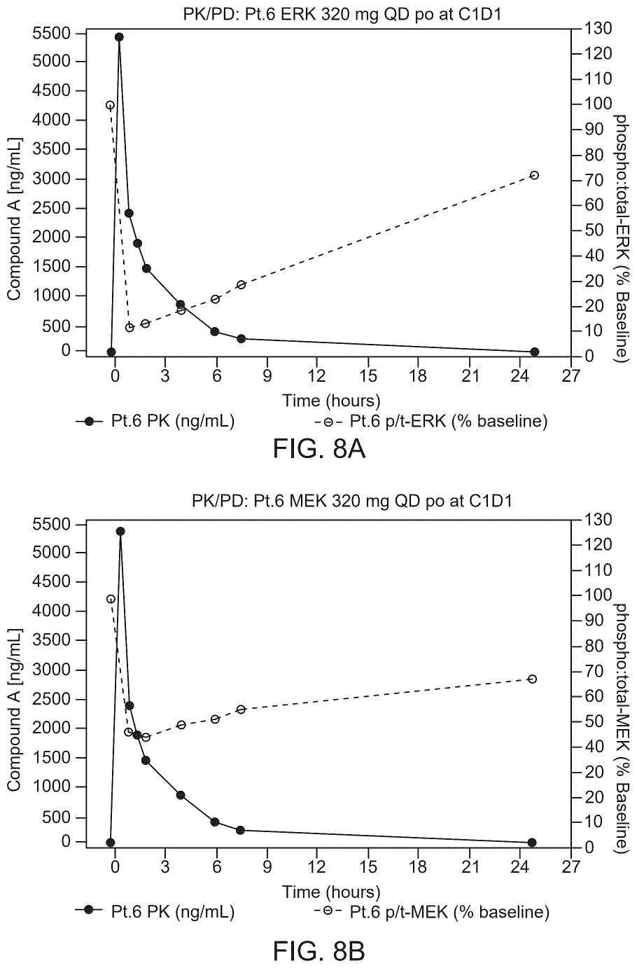

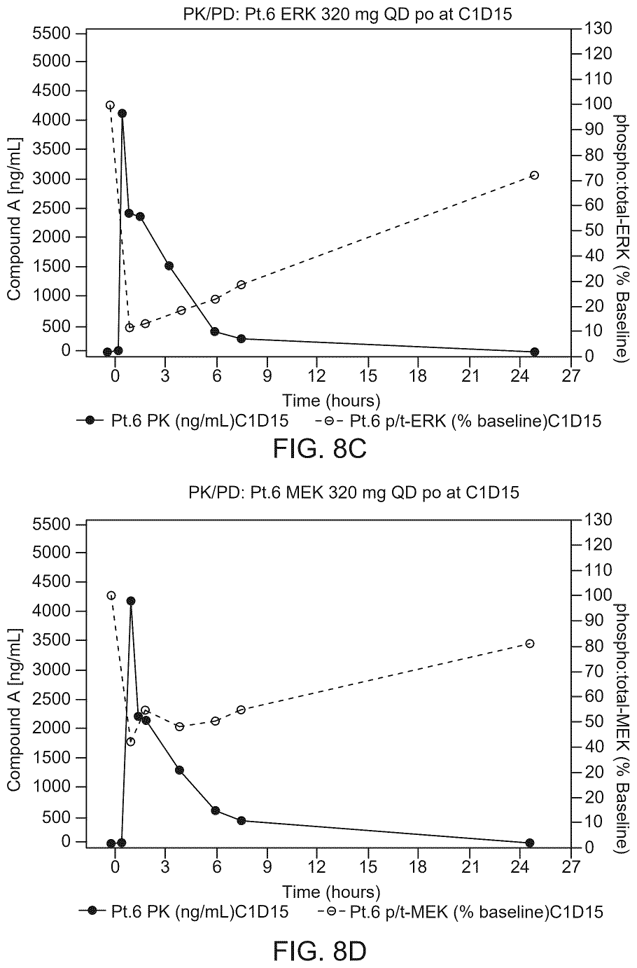

Compound A was administered to patients to treat pancreatic cancer. FIGS. 7A-7D show the PK/PD curves for pERK and pMEK obtained from a patient diagnosed with KRAS-G12D pancreatic cancer received 320 mg of Compound A once daily, for 15 days in cycle 1. Table 1 provides the corresponding half-life measured at day 1 and day 15. FIG. 7A is a graph showing PK/PD (pERK) for Compound A from patient 5 after receiving 320 mg administered orally (po) once daily (QD) (cycle 1, day 1). FIG. 7B is a graph showing PK/PD (pMEK) for Compound A from patient 5 after receiving 320 mg administered orally (po) once daily (QD) (cycle 1, day 1). FIG. 7C is a graph showing PK/PD (pERK) for Compound A from patient 5 after receiving 320 mg administered orally (po) once daily (QD) (cycle 1, day 15). FIG. 7D is a graph showing PK/PD (pMEK) for Compound A from patient 10 after receiving 320 mg administered orally (po) once daily (QD) (cycle 1, day 15). FIGS. 8A-8D are PK/PD curves for pERK and pMEK obtained from a patient diagnosed with KRAS-G12V pancreatic cancer who received 320 mg of Compound A once daily, for 15 days in cycle 1. Table 5 provides the corresponding half-life measured at day 1 and day 15. FIG. 8A is a graph showing PK/PD (pERK) for Compound A from patient 6 after receiving 320 mg administered orally (po) once daily (QD) (cycle 1, day 1). FIG. 8B is a graph showing PK/PD (pMEK) for Compound A from patient 6 after receiving 320 mg administered orally (po) once daily (QD) (cycle 1, day 1). FIG. 8C is a graph showing PK/PD (pERK) for Compound A from patient 6 after receiving 320 mg administered orally (po) once daily (QD) (cycle 1, day 15). FIG. 8D is a graph showing PK/PD (pMEK) for Compound A from patient 6 after receiving 320 mg administered orally (po) once daily (QD) (cycle 1, day 15).

In one aspect, a method of treating pancreatic adenocarcinoma includes administering to the patient in need thereof Compound A in combination with, before or after, a gemcitabine-based regimen as provided in Table 1.

TABLE 1
Chemotherapy Regimen combinations with Compound A

| | | Gemcitabine-based Regimen |
|---|---|---|
| | | (if fixed dose infusion rate not |
| | | utilized, administer gemcitabine |
| Regimen | Compound A | 1,000 mg/m² over 30 minutes) |
| 1 | Compound A 320 mg | Gemcitabine 600-750 mg/m² IV on Days 1, |
| | QD (or 240 mg | 8, 15 (fixed dose infusion rate of 10 mg/m²/ |
| | reduced dose) PO | minute preferred) |
| | | Repeat every 28 days |
| 2 | Compound A 320 mg | Gemcitabine 600-750 mg/m² IV on Day 1 |
| | QD (or 240 mg | (fixed dose infusion rate of 10 mg/m2/ |
| | reduced dose) PO | min preferred) |
| | | Cisplatin 30 mg/m² IV over 60 minutes |
| | | on Day 1 |
| | | Repeat every 14 days |
| | | (The preferred doublet for tumors with |
| | | germline BRCA mutations) |
| 3 | Compound A 320 mg | Gemcitabine 600-750 mg/m² IV on Days 1 |
| | QD (or 240 mg | and 8 (fixed dose infusion rate of 10 mg/m²/ |
| | reduced dose) PO | minute preferred) |
| | | Capecitabine 1,500-1,800 mg/m²/day PO |
| | | divided twice daily on Days 1-14 |
| | | Repeat every 21 days |
| 4 | Compound A 320 mg | (dosing from ESPAC-4 in the adjuvant setting) |
| | QD (or 240 mg | Gemcitabine 1,000 mg/m2 IV over 30 minutes |
| | reduced dose) PO | weekly on Days 1, 8, and 156 |
| | | Capecitabine 1,660 mg/m2/day PO divided |
| | | twice daily on Days 1-216 |
| | | Repeat every 28 days |
| 5 | Compound A 320 mg | Good performance status: |
| | QD (or 240 mg | Paclitaxel protein-bound 100-125 mg/m² IV |
| | reduced dose) PO | on Days 1, 8, 15 |
| | | Gemcitabine 600-750 mg/m² IV on Days 1, 8, |
| | | 15 (fixed dose infusion rate of 10 mg/m²/min |
| | | preferred) |
| | | Repeat every 28 days |
| | | Average performance status: |
| | | Paclitaxel protein-bound 125-175 mg/m² IV |
| | | on Day 1 |
| | | Gemcitabine 600-750 mg/m² IV on Day 1 |
| | | (fixed dose infusion rate of 10 mg/m²/ |
| | | min preferred) |
| | | Repeat every 14 days |
| 6 | Compound A 320 mg | Gemcitabine 300-400 mg/m² IV on Days 4 |
| | QD (or 240 mg | and 11 (fixed dose infusion rate of 10 mg/ |
| | reduced dose) PO | m²/minute preferred) |
| | | Docetaxel 30-40 mg/m² IV on Days 4 and 11 |
| | | Capecitabine 1,000 mg/m²/day PO divided |
| | | twice daily on Days 1-14 |
| | | Repeat every 21 days |
| 7 | Compound A 320 mg | Gemcitabine 600-750 mg/m² IV on Day 1 |
| | QD (or 240 mg | (fixed dose infusion rate of 10 mg/m²/ |
| | reduced dose) PO | minute preferred) |
| | | Oxaliplatin 85 mg/m² IV over 2 hours on Day 1 |
| | | Repeat every 14 days |

Methods of treatment may include the administration of Compound A in combination with oxaliplatin and/or fluorouracil for the treatment of pancreatic adenocarcinoma in a patient in need thereof. In one aspect, a method of treating pancreatic adenocarcinoma includes administering to the patient in need thereof Compound A in combination with, before or after, a fluoropyrimidine-based regimen in Table 2A, a FOLFIRINOX regimen in Table 2B or Table 2C.

TABLE 2A
Chemotherapy Regimen combinations with Compound A

| Regimen | Compound A | Fluoropyrimidine-based Regimen |
|---|---|---|
| 1 | Compound A 320 mg | mFOLFOX 6 |
| | QD (or 240 mg | Oxaliplatin 85 mg/m² IV over 2 hours |
| | reduced dose) PO | on Day 1 |
| | | Leucovorin 400 mg/m² IV over 2 hours |
| | | on Day 18 |
| | | Fluorouracil 400 mg/m² IV bolus on Day 18, |
| | | then fluorouracil 2,400 mg/m2 IV continuous |
| | | infusion over 46 hours |
| | | Repeat every 14 days |
| 2 | Compound A 320 mg | XELOX or CapeOx |
| | QD (or 240 mg | Capecitabine 1,500-1,800 mg/m² PO divided |
| | reduced dose) PO | twice daily on Days 1-14, then |
| | | Oxaliplatin 85-100 mg/m² IV over 2 hours |
| | | on Day 1 |
| | | Repeat every 21 days |
| 3 | Compound A 320 mg | FOLFIRINOX |
| | QD (or 240 mg | Oxaliplatin 75-85 mg/m² IV over 2 hours |
| | reduced dose) PO | on Day 1 |
| | | Irinotecan 125-180 mg/m² IV over 90 minutes |
| | | on Day 1 |
| | | Leucovorin 400 mg/m² IV over 2 hours on |
| | | Day 17 |
| | | Fluorouracil 400 mg/m² IV bolus on Day 17, |
| | | then fluorouracil 2,400 mg/m² IV continuous |
| | | infusion over 46 hours |
| | | Repeat every 14 days |
| 4 | Compound A 320 mg | Liposomal irinotecan (Onivyde ®) plus |
| | QD (or 240 mg | 5-fluorouracil |
| | reduced dose) PO | Liposomal irinotecan 70 mg/m² IV over 90 |
| | | minutes on Day 1 (For patients with known |
| | | homozygous UGT1A1*28 allele reduce the |
| | | initial starting dose to 50 mg/m²) |
| | | Leucovorin 400 mg/m² IV over 2 hours on |
| | | Day 1 |
| | | Fluorouracil 400 mg/m² IV bolus on Day 1, |
| | | then Fluorouracil 2,400 mg/m² IV continuous |
| | | infusion over 46 hours |
| | | Repeat every 14 days |

TABLE 2B
FOLFIRINOX (irinotecan, oxaliplatin and fluorouracil) in combination
with Compound A (e.g., 320 mg QD PO or 240 mg QD PO)

| Day | Drug | Dose | | Route | Diluent & Rate |
|---|---|---|---|---|---|
| Day 1 | Glucose 5% | 500 | ml | Infusion | Fast Running for Line Flush |
| | Ondansetron | 8 | mg | Oral | |
| | Dexamethasone | 8 | mg | Oral/Slow bolus/15 min infusion | |
| | Oxaliplatin | 85 | mg/m² | IV infusion | 250 ml glucose 5% over 2 hours |
| | Calcium Folinate | 200 | mg/m² | IV Infusion | 250 ml Glucose 5% over 2 hours. |
| | (Folinic Acid) | | | | After 30 min. start Irinotecan |
| | Irinotecan | 180 | mg/m² | IV Infusion | Over 90 minutes in 250 ml Glucose |
| | | | | | 5% alongside folinic acid |
| | Glucose 5% | | | | 250 ml line flush |
| | 5 Fluorouracil | 400 | mg/m² | IV bolus | Over 5 minutes |
| | 5 Fluorouracil | 2400 | mg/m² | via infusor | Sodium chloride 0.9% over |
| | | | | device | 46 hours |

An example of a modified FOLFIRINOX dosing schedule is provided in Table 8C. Oxaliplatin is administered in 250 mL glucose 5% over 2 hours. This is infused concurrently with calcium folinate in 250 mL glucose 5% over 2 hours. The line should then be flushed with glucose 5%.

TABLE 2C
modified FOLFIRINOX (14 day cycle frequency,
for up to 12 cycles) in combination with Compound
A (e.g., 320 mg QD PO or 240 mg QD PO)

| Day | Drug | Dose | | Route |
|---|---|---|---|---|
| 1 | Calcium folinate | 350 | mg | IV infusion |
| 1 | Oxaliplatin | 85 | mg/m2 | IV infusion |
| 1 | Irinotecan | 150 | mg/m2 | IV infusion |
| 1-2 | Fluorouracil | 2400 | mg/m2 | IV infusion |
| (46 hours) | | | | over 46 hours |

Methods of treatment may include the administration of Compound A in combination with chemoradiation for the treatment of pancreatic adenocarcinoma in a patient in need thereof. In one aspect, a method of treating pancreatic adenocarcinoma includes administering to the patient in need thereof Compound A in combination with, before or after, chemoradiation-based regimen as provided in Table 3.

TABLE 3
Chemoradiation Regimen combinations with Compound A

| Regimen | Compound A | Chemoradiation Regimen |
|---|---|---|
| 1 | Compound A 320 mg | Long course chemoradiation |
| | QD (or 240 mg | Total dose 50 Gy in 25 fractions or |
| | reduced dose) PO | 50.4 Gy in 28 fractions |
| | | Concurrent capecitabine 1,650 mg/m² |
| | | Concurrent gemcitabine 300-400 mg/m² IV given |
| | | at fixed dose infusion once weekly (if fixed |
| | | dose infusion rate of 10 mg/m²/minute not |
| | | utilized, administer gemcitabine over 30 minutes) |
| 2 | Compound A 320 mg | Short course chemoradiation |
| | QD (or 240 mg | Total dose 30 Gy in 10 fractions |
| | reduced dose) PO | Concurrent capecitabine 1,650 mg/m² PO in two |
| | | divided doses on each day of radiation or |
| | | Concurrent gemcitabine 300-400 mg/m² IV given |
| | | at fixed rate dose infusion once weekly (if fixed |
| | | dose infusion rate of 10 mg/m²/minute not |
| | | utilized, administer gemcitabine over 30 minutes) |
| 3 | Compound A 320 mg | Hypofractionated chemoradiation |
| | QD (or 240 mg | Total dose 60-67.5 Gy in 15 fractions |
| | reduced dose) PO | Concurrent capecitabine 1,650 mg/m² PO in |
| | | two divided doses on each day of radiation |
| | | Requires image guidance |
| 4 | Compound A 320 mg | Total dose 33-40 Gy in 5 fractions |
| | QD (or 240 mg reduced | Usually requires fiducials |
| | dose) PO | Requires daily image guidance |

The oral administration of various doses of atebimetinib (Compound A) administered orally (po) once daily (QD) for the treatment of pancreatic cancer patients was evaluated in a Phase 1 and a Phase 2a human clinical trial. Atebimetinib was administered to treat patients diagnosed with pancreatic cancer at doses including 240 mg po QD and 320 mg po QD, in combination with a modified gemcitabine+nab-paclitaxel (mGnP) regimen where the mGnP regimen is administered on Day 1 and Day 15 of each 28-day cycle as follows: nab-paclitaxel 125 mg/m² via IV infusion over 30 minutes, followed immediately by gemcitabine 1,000 mg/m² via IV infusion over 30 minutes. Results are discussed below.

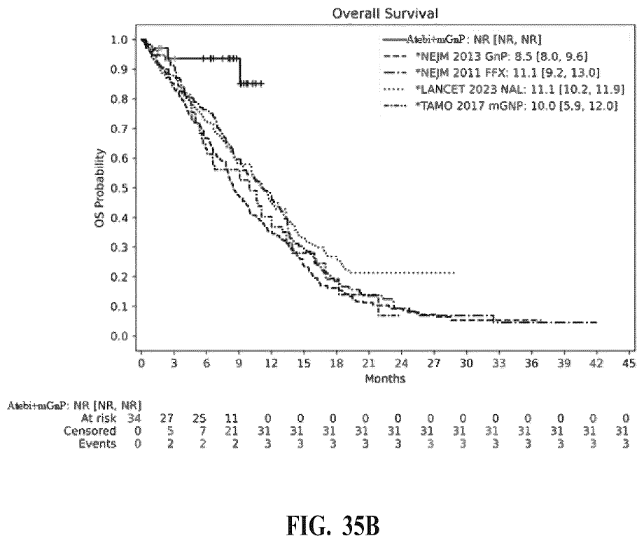

A 94% overall survival (OS) was observed at 6 months in first-line (1L) PDAC pancreatic cancer patients treated with 320 mg of atebimetinib administered po QD in combination with modified gemcitabine/nab-paclitaxel (mGnP) (i.e., 1,000 mg/m² gemcitabine+125 mg/m² nab-paclitaxel on days 1 & 15 of a treatment cycle, once every 4 weeks) (N=34). FIG. 35A is a graph of overall survival (OS) probability by month in a human clinical trial of atebimetinib (320 mg QD) in the treatment of first line pancreatic (PDAC) patients, in combination with modified gemcitabine and nab-paclitaxel treatment, compared to third party published benchmark OS data obtained from different patients in the Phase 3 MPACT clinical trial. The data in FIG. 35A shows an 8-month OS of 94%, based on interim data collection from the 320 mg intent-to-treat population (N=34). This represents the primary Phase 2 population enrolled as part of the Simon two-stage design from the ongoing Phase 1/2a trial of atebimetinib. The benchmark 6-month OS for the standard of care treatment in this population (full dose and schedule GnP) is estimated as 67% and estimated at about 55% at 8-month OS data, based on the published results from the MPACT study (Pivotal Ph3 Study MPACT 2013 NEJM (PMID: 24131140) per 2024 JAMA Nichetti, et al. 7(1):e2350756). The median OS was not yet reached at the data cutoff date. FIG. 35B is a graph of the overall survival (OS) data from patients treated with atebimetinib in FIG. 35A compared to third party published benchmark OS data obtained from other published clinical trial data. No head-to-head clinical trial has been conducted evaluating atebimetinib and other candidates or products. Differences exist between trial designs, subject characteristics and other factors, and caution should be exercised when comparing data across studies. The data shown in the lower line of FIG. 35B is a Kaplan-Meier (KM) Plot of the Pivotal Ph3 Study MPACT 2013 NEJM (PMID: 24131140) per 2024 JAMA Nichetti, et al. 7(1):e2350756, incorporated herein by reference in its entirety.

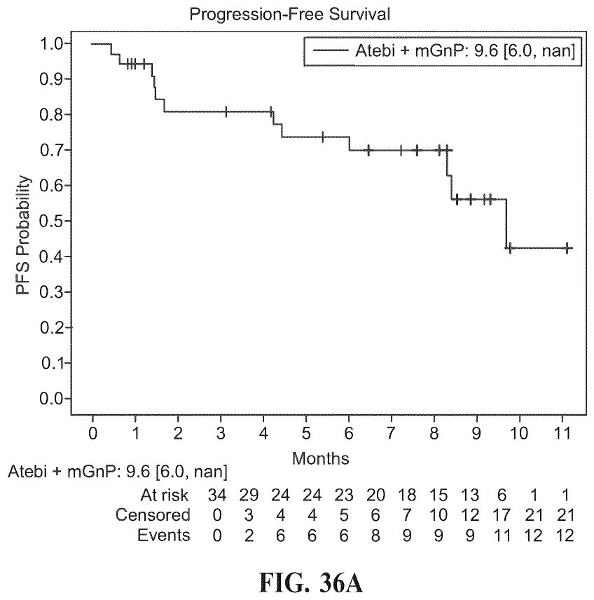

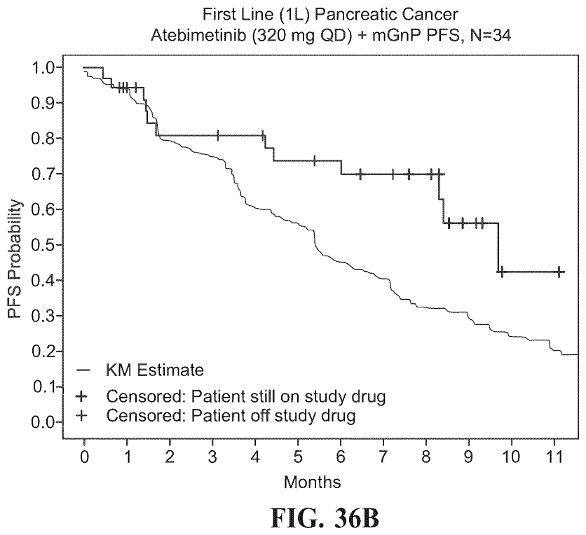

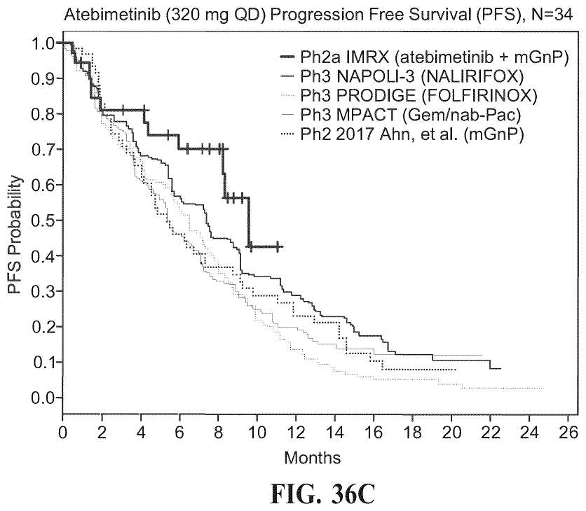

In addition, a 72% progression-free survival (PFS) was observed at 6 months in first-line (1L) pancreatic cancer patients treated with atebimetinib+mGnP at the 320 mg dose level (N=34). FIG. 36A is a graph showing the progression free survival probability by month in a human clinical trial of atebimetinib (320 mg QD) in the treatment of first line pancreatic (PDAC) patients, in combination with modified gemcitabine and nab-paclitaxel treatment, based on interim data collection from the 320 mg intent-to-treat population (N=34). This represents the primary Phase 2 population enrolled as part of the Simon two-stage design from the ongoing Phase 1/2a trial of atebimetinib. The benchmark 6-month PFS for the standard of care treatment in this population (full dose and schedule GnP) is 44% and about 32% at 8-month PFS (extrapolated from reconstructed plots per 2024 JAMA Nichetti, et al. 7(1):e2350756]: (1.) MPACT 2013 NEJM (PMID: 24131140) N=431 [˜32%]). FIG. 36B is a graph showing the progression free survival (PFS) probability by month in a human clinical trial of atebimetinib (320 mg QD) combined with modified gemcitabine and nab-paclitaxel treatment in the treatment of first line pancreatic (PDAC) patients, as shown in FIG. 36A, compared to third party published benchmark PFS probability data obtained from different patients in the Phase 3 MPACT clinical trial (2024 JAMA Nichetti, et al. 7(1):e2350756]: (1.) MPACT 2013 NEJM (PMID: 24131140), incorporated herein by reference in its entirety). FIG. 36C is a graph of the progression free survival (PFS) data from patients treated with atebimetinib in FIG. 36A compared to third party published benchmark PFS probability data obtained from other published clinical trial data. The 8 mo PFS benchmark data is extrapolated from reconstructed plots per 2024 JAMA Nichetti, et al. 7(1):e2350756]: (1.) MPACT 2013 NEJM (PMID: 24131140) N=431 [˜32%], (2.) PRODIGE 4/ACCORD 11 2011 NEJM (PMID: 21561347) N=171 [˜34%], (3.) NAPOLI 3 2023 LANCET (PMID: 37708904) N=383 [˜44%], (4.) Ph2—2017 Ahn, et al. (PMID: 28203300) N=57 [˜36%]. No head-to-head clinical trial has been conducted evaluating atebimetinib and other candidates or products in FIG. 35A, FIG. 35B, FIG. 36B and FIG. 36C. Differences exist between trial designs, subject characteristics and other factors, and caution should be exercised when comparing data across studies.

In addition, data was obtained from treating PDAC patients (N=6) with 240 mg atebimetinib QD and mGNP (1,000 mg/m² (Gem)+125 mg/m² (nab-Pac) days 1 & 15, every 4 weeks), resulting in a median PFS of 7.1 months, and survival probability of about 20% at 8 months (estimated using a Cox proportional hazards regression model, with Kaplan-Meier methods were used to visualize time-to-event data). This data was based on interim data collection from the 240 mg (N=6) intent-to-treat populations, from an ongoing Phase 1/2a trial of atebimetinib.

The median PFS for the ongoing clinical trial with atebimetinib with mGnP has not yet reached at the data cutoff date. An overall response rate (ORR) of 39% and a disease control rate (DCR) of 81% were observed in response evaluable patients at both the 240 and 320 mg dose levels of atebimetinib+mGnP (N=36), including many patients with deepening, durable regressions and multiple examples of individual lesions rendered undetectable. Atebimetinib continued to demonstrate a markedly favorable tolerability profile in combination with mGnP. No Grade 3+ events were observed in a majority of the adverse event categories commonly observed with standard of care chemotherapy in first line pancreatic cancer.

The combination of atebimetinib and mGnP showed a markedly favorable tolerability profile. Safety data for the clinical trial of atebimetinib and mGnP described above is presented in Table 4 below, in comparison to data obtained from the following third party published pivotal trials: (1.) MPACT 2013 NEJM (PMID: 24131140) N=431, (2.) PRODIGE 4/ACCORD 11 2011 NEJM (PMID: 21561347) N=171, (3.) NAPOLI 3 2023 LANCET (PMID: 37708904) N=383, (4.) FFX pivotal study follow up (PMID: 27765912) (NR=not reported or not clearly reported). Not all pivotal trials reported on all AE's or used fully consistent terminology.

TABLE 4
Safety Data

| | | FOLFIRINOX | | Atebimetinib + |
|---|---|---|---|---|
| | Gem/nab-Pac | (²PRODIGE/ | NALIRIFOX | mGnP |
| | (¹MPACT; | ACCORD 11; | (³NAPOLI 3; | (320 mg atebi-; |
| STUDY: | N = 431) | N = 171) | N = 383) | N = 34) |
| Adverse Event (AE) | Grade ≥ 3 | Grade ≥ 3 | Grade ≥ 3 | Grade ≥ 3 |
| | Incidence (%) | Incidence (%) | Incidence (%) | Incidence (%) |
| Neutropenia | 38% | 45.7% | 14.1% | 15% ^{a} |
| Fatigue | 17% | 23.6% | 6.2% | 6% |
| Diarrhea | 6% | 12.7% | 20.3% | 0% |
| Sensory Neuropathy | 17% | 9% | 3.2% | 0% |
| Leukopenia | 31% | NR | NR | 0% |
| Vomiting | NR | 14.5% | 7% | 3% |
| Febrile Neutropenia | 3% | 5.4% | NR | 3% |
| Thrombocytopenia | 13% | 9.1% | NR | 0% |
| Anemia | 13% | 7.8% | 10.5% | 24% ^{a} |
| Hypokalemia | NR | NR | 15.1% | 3% |
| Nausea | NR | NR | 11.9% | 3% |

^{a}= only AE groups in this atebi + mGnP arm (N = 34) that reached ≥ 10% Gr3 event level. For Anemia, all Grade 3, not SAE's, older patient population vs. historical benchmarks Neutropenia: Neutropenia, Neutrophil Count Decreased Sensory Neuropathy: Peripheral Sensory Neuropathy, Neuropathy Peripheral No Gr 5 events; Patients received combination of 320 mg atebi + mGnP (N = 34)

To date, no head-to-head clinical trial has been conducted evaluating atebimetinib and other candidates or products.

In some embodiments, Compound A may be formulated as a pharmaceutical composition using a pharmaceutically acceptable excipients and administered by a variety of routes. In some embodiments, such compositions are for oral administration. Such pharmaceutical compositions and processes for preparing them are well known in the art. (See, e.g., Remington: The Science and Practice of Pharmacy, L. V. Allen, Editor, 22nd Edition, Pharmaceutical Press, 2012). In a particular embodiment, the pharmaceutical composition comprises 4-((dimethylamino)methyl)-3-(2-fluoro-3-((N-methylsulfamoyl)amino)benzyl)-2-oxo-2H-chromen-7-yl dimethylcarbamate or a pharmaceutically acceptable salt or hydrate thereof, and a pharmaceutically acceptable carrier.

In some embodiments, to prepare a pharmaceutical composition for oral delivery, 0.1 mg to 120 mg of a compound of an embodiment is mixed with 750 mg of starch. The mixture is incorporated into an oral dosage unit, such as a hard gelatin capsule, or 0.1 mg to 120 mg of compound is granulated with binder solution such as starch solution along with suitable diluents such as microcrystalline cellulose or like, disintegrants such as crosscarmellose sodium, dry the resultant mixture and add lubricant and compress into tablet which is suitable for oral administration.

In certain embodiments, the present application is directed to a pharmaceutical composition comprising an active pharmaceutical ingredient. In certain embodiments, the pharmaceutical composition comprises a compound as disclosed herein as the active pharmaceutical ingredient (API) and a pharmaceutically acceptable carrier comprising one or more excipients. In some embodiments, the pharmaceutical composition optionally further comprises an additional therapeutic compound (i.e., agent) with the pharmaceutically acceptable carrier. The pharmaceutical composition can be a medicament.

Pharmaceutically acceptable carriers include those known in the art. The choice of a pharmaceutically acceptable carrier can depend, for example, on the desired route of administration of the composition. A pharmaceutical composition (preparation) can be administered to a subject by any of a number of routes of administration including, for example, parenteral administration (e.g., intravenously, subcutaneously, or intramuscularly), oral administration (for example, tablets, and capsules); absorption through the oral mucosa (e.g., sublingually) or transdermally (for example as a patch applied to the skin) or topically (for example, as a cream, ointment or spray applied to the skin).

In certain embodiments, the present application is directed to a pharmaceutical composition comprising an active pharmaceutical ingredient. In certain embodiments, the pharmaceutical composition comprises a compound as disclosed herein as the active pharmaceutical ingredient (API) and a pharmaceutically acceptable carrier comprising one or more excipients. In some embodiments, the pharmaceutical composition optionally further comprises an additional therapeutic compound (i.e., agent) with the pharmaceutically acceptable carrier. In some embodiments, the pharmaceutical composition can be a medicament.

In some embodiments, pharmaceutical compositions comprising Compound A or pharmaceutically acceptable salts thereof can be formulated for oral administration. For example, a compound provided herein can be combined with suitable compendial excipients to form an oral unit dosage form, such as a capsule or tablet, containing a target dose of Compound A. The drug product can be prepared by first manufacturing Compound A as an active pharmaceutical ingredient (API), followed by roller compaction/milling with intragranular excipients and blending with extra granular excipients. A Drug Product can contain Compound A as the API and excipient components in a tablet in a desired dosage strength of Compound A. The blended material can be compressed to form tablets and then film coated. The excipients can be selected from materials appropriate for inclusion in a pharmaceutical composition for an intended purpose and route of delivery including providing a desired manufacturing and stability properties and/or desired in vivo characteristics or other properties to the pharmaceutical composition. In some embodiments, the pharmaceutical composition can include Compound A as the API in combination with a filler (e.g., a form of microcrystalline cellulose), a dry binder or disintegrant (e.g., a cross-linked polymer), a glidant (e.g., colloidal silicon dioxide) and/or a lubricant (e.g., magnesium stearate). In some embodiments, the pharmaceutical composition can comprise a material such as an extended release or disintegrant involved in carrying or transporting the API pharmaceutical agent from one organ, or portion of the body, to another organ, or portion of the body of a subject, including materials to desirable control the absorption of the API in the intestine.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. For use in the methods of this disclosure, active compounds can be given per se or as a pharmaceutical composition containing, for example, 0.1 to 99.5% (or, for example, 0.5 to 90%) of an active ingredient in combination with a pharmaceutically acceptable carrier.

Methods of preparing these formulations or compositions include the step of bringing into association an active compound, such as a compound of the disclosure, with the carrier and, optionally, one or more accessory ingredients. In general, the formulations are prepared by uniformly and intimately bringing into association a compound of the present disclosure with liquid carriers, or finely divided solid carriers, or both, and then, if necessary, shaping the product.

To prepare solid dosage forms for oral administration, the active ingredient is mixed with one or more pharmaceutically acceptable carriers, such as sodium citrate or dicalcium phosphate, and/or any of the following: (1) fillers or extenders, (2) binders, (3) humectants, (4) disintegrating agents, (5) solution retarding agents, (6) absorption accelerators, (7) wetting agents, (8) absorbents, (9) lubricants, (10) complexing agents, and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using suitable excipients. The pharmaceutical compositions according to the present disclosure may contain conventional pharmaceutical carriers and/or auxiliary agents. In some embodiments, the pharmaceutical compositions according to the present disclosure may contain conventional carrier agents including a binder, a lubricant and/or a glidant selected from those products and materials generally used in pharmaceutical industry for preparation of pharmaceutical compositions for an intended route of administration.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

In some embodiments, an oral dosage form of Compound A is provided, such as the oral dosage form disclosed in Example 14. In some embodiments, an oral dosage form such as a tablet or capsule can contain Compound A or a pharmaceutically acceptable salt thereof as the API and one or more excipients such as a dry binder, tablet disintegrant, an absorbent, filler or diluent, a lubricant, anti-adherent, binder, thickening agent, viscosity-increasing agent, coating agent and film former. In some embodiments, an oral dosage form such as a tablet or capsule can contain 20 mg to 160 mg of Compound A or a pharmaceutically acceptable salt thereof as the API and one or more intra-granulation excipients such as a dry binder, tablet disintegrant, an absorbent, filler or diluent, a lubricant, anti-adherent, binder, thickening agent, viscosity-increasing agent, coating agent and film former. In some embodiments, an oral dosage form such as a tablet or capsule can contain 20 mg, 120 mg or 160 mg of Compound A or a pharmaceutically acceptable salt thereof as the API and one or more intra-granulation excipients such as a dry binder, tablet disintegrant, an absorbent, filler or diluent, a lubricant, anti-adherent, binder, thickening agent, viscosity-increasing agent, coating agent and film former and one or more extra granulation excipients such as additional Compound A API granulate and a lubricant or film forming agent.

In some embodiments, an oral dosage form such as a tablet or capsule can contain 20 mg, 120 mg or 160 mg of Compound A or a pharmaceutically acceptable salt thereof as the API and one or more intra-granulation excipients such as microcrystalline cellulose, crospovidone Type A, and hydroxypropyl cellulose, and one or more extra-granulation excipients such as API granulate of Compound A and magnesium stearate. In some embodiments, an oral dosage form such as a tablet or capsule can contain about 20% w/w of Compound A or a pharmaceutically acceptable salt thereof as the API and about 60-70% w/w microcrystalline cellulose, about 5% w/w crospovidone Type A, and about 10% w/w hydroxypropyl cellulose, and one or more extra-granulation excipients such as API granulate of Compound A (95-99.5% w/w) and magnesium stearate (0.5-5% w/w).

Techniques and compositions for making dosage forms useful in the methods described herein are described in the following references, all incorporated by reference herein: Modern Pharmaceutics, 4th Ed., Chapters 9 and 10 (Banker & Rhodes, editors, 2002); Lieberman et al., Pharmaceutical Dosage Forms: Tablets (1989); and Ansel, Introduction to Pharmaceutical Dosage Forms 8th Edition (2004).

In certain aspects, therapeutic methods or uses are provided herein for treating, preventing, or ameliorating a disease or condition in a subject.

In some embodiments, atebimetinib (Compound A) is administered to treat a cancer harboring a RAF or RAS mutation at a dose that provides Deep Cyclic Inhibition (DCI) of the MAPK pathway. In some embodiments, methods of administering atebimetinib provide a short-lived dual MEK inhibitor (MEKi) that is resistant to CRAF-bypass and has a unique pulsatile pharmacokinetic and pharmacodynamic mechanism (e.g., DCI MEKi). For example, in some embodiments, methods of treating a cancer can comprise administering a dose of atebimetinib at a dose and dose interval (e.g., 240-320 mg QD that exhibits high Cmax and near-zero drug trough, approximating a 2 h plasma half-life and >90% inhibition of pERK) and provides adequate anti-tumor activity while not exceeding the MTD (e.g. Cmax levels over 2,000 ng/mL for ˜1 μM drug free fraction, a median plasma half life of about 2 hours, with ˜90% pharmacodynamic inhibition of pERK).

Some embodiments relate to the use of a compound or composition described herein to treat, prevent, or ameliorate cancer associated with an oncogenic RAS mutation. Some embodiments relate to the use of a compound or composition as described herein to treat cancer characterized by expressing a mutant form of RAS protein in a subject. Some embodiments relate to the use of a compound or composition as described herein to inhibit the proliferation of a cell having a RAS mutation. Some embodiments relate to the use of a compound or composition as described herein to induce apoptosis in a cell having a RAS mutation. Some embodiments relate to the use of a compound or composition as described herein to inhibit the progression of cancer that is characterized by expressing a mutant form of RAS protein in a subject. In some embodiments, the RAS protein is a KRAS protein. In some embodiments, the RAS protein is an NRAS protein. In some embodiments, the RAS protein is an HRAS protein.

In some embodiments, patients are selected for treatment with Compound A based on the presence of RAS mutation such as a KRAS G12C mutation in plasma or tumor specimens. If no mutation is detected in a plasma specimen, test tumor tissue. Additional information on FDA-approved tests for the detection of a KRAS G12C mutation is available at: https://www.fda.gov/CompanionDiagnostics.

In some embodiments, methods of treating pancreatic cancer having a RAS mutation are provided, wherein the RAS mutation is a KRAS G12D or G12V mutation or a NRAS Q61L or Q61K mutation. In some embodiments, the cancer has a KRAS G12D mutation. In some embodiments, the cancer has a KRAS G12V mutation. In some embodiments, the cancer has a NRAS Q61L mutation. In some embodiments, the cancer has a NRAS Q61K mutation.

In some embodiments, the pancreatic cancer is selected from the group consisting of: NRAS-mutated melanoma, KRAS-mutated PDAC, KRAS-mutated NSCLC and KRAS-mutated, and APC-wildtype CRC. In some embodiments, the cancer is NRAS-mutated melanoma. In some embodiments, the cancer is KRAS-mutated PDAC. In some embodiments, the cancer is KRAS-mutated NSCLC. In some embodiments, the cancer is KRAS-mutated CRC. In some embodiments, the cancer is APC-wildtype CRC. In some embodiments, the cancer is pancreatic cancer with a KRAS G12C mutation. In some embodiments, the cancer is pancreatic cancer with a G12D mutation. In some embodiments, the cancer is pancreatic cancer with a G12V mutation. In some embodiments, the cancer is colorectal cancer with a NRAS Q61L mutation. In some embodiments, the cancer is colorectal cancer with a NRAS Q61K mutation. In some embodiments, the cancer is colorectal cancer with a KRAS G12D mutation. In some embodiments, the cancer is melanoma with a NRAS Q61R mutation. In some embodiments, the cancer is melanoma with a KRAS G12S mutation. In some embodiments, the cancer melanoma with a BRAF V600E mutation. In some embodiments, the cancer is non-small cell lung cancer (NSCLC) with a KRAS G12S mutation.

In some embodiments, RAS mutations can be detected by next generation sequencing (NGS) techniques. For example, molecular diagnostics of HRAS/KRAS and NRAS targeted gene panels can be obtained by NGS techniques. Next generation sequencing of amplification products can be performed on the Ion Torrent Personal Genome Machine and analyzed with the Torrent Suite Software. Reference DNA sequences used for these genes can be found at https://www.ncbi.nlm.nih.gov/refseq/rsg/. This mutation panel is designed to detect targeted mutations only. Confirmation of these mutations is performed by traditional or real time PCR followed by Sanger sequencing, fluorescent melting curve analysis and/or pyrosequencing (CPT codes: 81210 (BRAF), 81275 (KRAS codons 12 and 13 in exon 2), 81276 (KRAS codon 61 in exon 3), 81403 (HRAS), 81311 (NRAS)).

In some embodiments, patients are selected for treatment with Compound A based on the presence of RAS mutation such as a KRAS G12C mutation in plasma or tumor specimens. If no mutation is detected in a plasma specimen, test tumor tissue. Additional information on FDA-approved tests for the detection of a KRAS G12C mutation is available at: https://www.fda.gov/CompanionDiagnostics. FDA approved diagnostics for identifying patients with a RAS-mutated cancer include various companion diagnostics. For example, RAS mutations can be detected in pancreatic cancer using a companion diagnostic with next-generation sequencing to analyze the genomic profile of the patient's RAS gene mutation status such as the FoundationOne CDx (Foundation Medicine), or other multi-gene NGS panels for detecting RAS mutations, often performed on biopsy tissue from solid tumors or liqui biopsy tests for circulating tumor DNA (ctDNA). In some embodiments, RAS mutations can be detected by next generation sequencing (NGS) techniques. For example, molecular diagnostics of HRAS/KRAS and NRAS targeted gene panels can be obtained by NGS techniques. Next generation sequencing of amplification products can be performed on the Ion Torrent Personal Genome Machine and analyzed with the Torrent Suite Software. Reference DNA sequences used for these genes can be found at https://www.ncbi.nlm.nih.gov/refseq/rsg/. This mutation panel is designed to detect targeted mutations only. Confirmation of these mutations is performed by traditional or real time PCR followed by Sanger sequencing, fluorescent melting curve analysis and/or pyrosequencing (CPT codes: 81210 (BRAF), 81275 (KRAS codons 12 and 13 in exon 2), 81276 (KRAS codon 61 in exon 3), 81403 (HRAS), 81311 (NRAS)).

### Definitions

The term “pharmaceutical composition,” as used herein, refers to a mixture of a compound disclosed herein with other chemical components, such as diluents or carriers. The pharmaceutical composition facilitates administration of the compound to an organism. Multiple techniques of administering a compound exist in the art including, but not limited to, oral, injection, aerosol, parenteral, and topical administration. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like.

The term “carrier,” as used herein, refers to a chemical compound that facilitates the incorporation of a compound into cells or tissues. For example dimethyl sulfoxide (DMSO) is a commonly utilized carrier as it facilitates the uptake of many organic compounds into the cells or tissues of an organism.

The term “diluent,” as used herein, refers to chemical compounds diluted in water that will dissolve the compound of interest as well as stabilize the biologically active form of the compound. Salts dissolved in buffered solutions are utilized as diluents in the art. One commonly used buffered solution is phosphate buffered saline because it mimics the salt conditions of human blood. Since buffer salts can control the pH of a solution at low concentrations, a buffered diluent rarely modifies the biological activity of a compound.

The term “physiologically acceptable,” as used herein, refers to a carrier or diluent that does not abrogate the biological activity and properties of the compound.

As used herein, an “excipient” refers to an inert substance that is added to a pharmaceutical composition to provide, without limitation, bulk, consistency, stability, binding ability, lubrication, disintegrating ability etc., to the composition. A “diluent” is a type of excipient.

As used herein, “alkyl” refers to a straight or branched hydrocarbon chain fully saturated (no double or triple bonds) hydrocarbon group. The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 10 carbon atoms. The alkyl group could also be a lower alkyl having 1 to 5 carbon atoms. The alkyl group of the compounds may be designated as “C₁-C₄ alkyl” or similar designations. By way of example only, “C₁-C₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, ethenyl, propenyl, butenyl, and the like.

The term “about,” as used herein, refers to a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length. When a value is preceded by the term about, the component is not intended to be limited strictly to that value, but it is intended to include amounts that vary from the value.

[The term “pharmaceutically acceptable carrier” or “pharmaceutically acceptable excipient,” as used herein, includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety.

The term “pharmaceutically acceptable salt,” as used herein, refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), sulfuric acid, nitric acid, phosphoric acid and the like. Pharmaceutical salts can also be obtained by reacting a compound with an organic acid such as aliphatic or aromatic carboxylic or sulfonic acids, for example acetic, succinic, lactic, malic, tartaric, citric, ascorbic, nicotinic, methanesulfonic, ethanesulfonic, p-toluenesulfonic, salicylic or naphthalenesulfonic acid. Pharmaceutical salts can also be obtained by reacting a compound with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, C1-C7 alkylamine, cyclohexylamine, triethanolamine, ethylenediamine, and salts with amino acids such as arginine, lysine, and the like.

The term “subject,” as used herein, refers to a human or a non-human mammal, e.g., a dog, a cat, a mouse, a rat, a cow, a sheep, a pig, a goat, a non-human primate or a bird, e.g., a chicken, as well as any other vertebrate or invertebrate.

The term “first line treatment” for cancer refers to the initial treatment administered to a patient after diagnosis or previously untreated cancer.

The term “second line treatment” for cancer refers to the second treatment administered to a patient after treatment of the cancer with the first line treatment, for example after the first line treatment for a cancer has failed to work (e.g. after disease progression or stable disease determination), stopped being effective or caused intolerable side effects. Second line treatment may involve a change to a different drug therapy or treatment regimen to treat or manage the cancer when the first line treatment is no longer considered the most beneficial treatment for the treatment of the cancer in the patient.

The term “subsequent line treatment” for cancer refers to the second or later line of treatment administered to a patient after treatment of the cancer with the second or immediately prior line treatment, for example after the earlier line treatment for a cancer has failed to work (e.g. after disease progression or stable disease determination), stopped being effective or caused intolerable side effects. Subsequent line treatment may involve a change to a different drug therapy or treatment regimen to treat or manage the cancer when the earlier line treatment is no longer considered the most beneficial treatment for the treatment of the cancer in the patient.

### Additional Embodiments

Accordingly, some aspects described herein relate to the following numbered alternatives:
1. A method of treating pancreatic ductal adenocarcinoma (PDAC) comprising the steps of administering to a patient in need thereof: oral administration of 240 mg of Compound A once per day, in combination with a modified gemcitabine+nab-paclitaxel (mGnP) regimen where the mGnP regimen is administered on Day 1 and Day 15 of each 28-day cycle as follows: nab-paclitaxel 125 mg/m2 via IV infusion over 30 minutes, followed immediately by gemcitabine 1000 mg/m2 via IV infusion over 30 minutes.
2. A use of 240 mg of Compound A administered once per day to treat pancreatic ductal adenocarcinoma (PDAC), in combination with a modified gemcitabine+nab-paclitaxel (mGnP) regimen where the mGnP regimen is administered on Day 1 and Day 15 of each 28-day cycle as follows: nab-paclitaxel 125 mg/m2 via IV infusion over 30 minutes, followed immediately by gemcitabine 1000 mg/m2 via IV infusion over 30 minutes.
3. A method of treating pancreatic ductal adenocarcinoma (PDAC) comprising the steps of administering to a patient in need thereof: oral administration of 320 mg of Compound A once per day, in combination with a modified gemcitabine+nab-paclitaxel (mGnP) regimen where the mGnP regimen is administered on Day 1 and Day 15 of each 28-day cycle as follows: nab-paclitaxel 125 mg/m2 via IV infusion over 30 min, followed immediately by gemcitabine 1000 mg/m2 via IV infusion over 30 min.
4. A use of 320 mg of Compound A once per day to treat pancreatic ductal adenocarcinoma (PDAC), in combination with a modified gemcitabine+nab-paclitaxel (mGnP) regimen where the mGnP regimen is administered on Day 1 and Day 15 of each 28-day cycle as follows: nab-paclitaxel 125 mg/m2 via IV infusion over 30 min, followed immediately by gemcitabine 1000 mg/m2 via IV infusion over 30 min.
5. A method of treating pancreatic ductal adenocarcinoma (PDAC) comprising the steps of administering to a patient in need thereof: oral administration of 240 mg of Compound A once per day, in combination with a modified FOLFIRINOX (mFFX) regimen where the mFFX regimen is administered on Day 1 and Day 15 of each 28-day cycle for up to 6 cycles, as follows: oxaliplatin 85 mg/m2 via IV infusion over 2 hours, folinic acid (leucovorin) 400 mg/m2 via IV infusion over 2 hours, irinotecan 150 mg/m2 via IV infusion over 90 minutes, and fluorouracil 2400 mg/m2 continuous IV infusion given over 46 hours beginning Day 1 and ending on Day 3.
6. A use of 240 mg of Compound A once per day to treat pancreatic ductal adenocarcinoma (PDAC), in combination with a modified FOLFIRINOX (mFFX) regimen where the mFFX regimen is administered on Day 1 and Day 15 of each 28-day cycle for up to 6 cycles, as follows: oxaliplatin 85 mg/m2 via IV infusion over 2 hours, folinic acid (leucovorin) 400 mg/m2 via IV infusion over 2 hours, irinotecan 150 mg/m2 via IV infusion over 90 minutes, and fluorouracil 2400 mg/m2 continuous IV infusion given over 46 hours beginning Day 1 and ending on Day 3.
7. A method of treating pancreatic ductal adenocarcinoma (PDAC) comprising the steps of administering to a patient in need thereof: oral administration of 320 mg of Compound A once per day, in combination with a modified FOLFINOX (mFFX) regimen where the mFFX regimen is administered on Day 1 and Day 15 of each 28-day cycle for up to 6 cycles, as follows: oxaliplatin 85 mg/m2 via IV infusion over 2 hours, folinic acid (leucovorin) 400 mg/m2 via IV infusion over 2 hours, irinotecan 150 mg/m2 via IV infusion over 90 minutes, and fluorouracil 2400 mg/m2 continuous IV infusion given over 46 hours beginning Day 1 and ending on Day 3.
8. A use of 320 mg of Compound A once per day to treat pancreatic ductal adenocarcinoma (PDAC), in combination with a modified FOLFIRINOX (mFFX) regimen where the mFFX regimen is administered on Day 1 and Day 15 of each 28-day cycle for up to 6 cycles, as follows: oxaliplatin 85 mg/m2 via IV infusion over 2 hours, folinic acid (leucovorin) 400 mg/m2 via IV infusion over 2 hours, irinotecan 150 mg/m2 via IV infusion over 90 minutes, and fluorouracil 2400 mg/m2 continuous IV infusion given over 46 hours beginning Day 1 and ending on Day 3.
9. A method reducing CA-19-9 in a patient diagnosed with pancreatic cancer, the method comprising administering atebimetinib (Compound A)

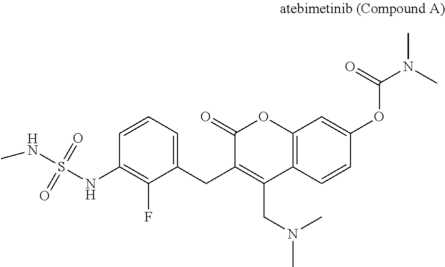

or a pharmaceutically acceptable salt thereof to the patient in need thereof once per day (QD) at a dose providing 320 mg of atebimetinib to the patient in need thereof.
10. The method of alternative 9, wherein the pancreatic cancer has a KRAS mutation.
11. The method of alternative 10, wherein the pancreatic cancer has a G12 KRAS mutation.
12. The method of alternative 10, wherein the pancreatic cancer has a G12D or G12V KRAS mutation.
13. The method of alternative 9, wherein the atebimetinib is administered in combination with a chemotherapy to treat the pancreatic cancer, wherein the chemotherapy consists of a modified gemcitabine and nab-paclitaxel chemotherapy (mGnP) or a modified FOLFIRINOX chemotherapy (mFFX), wherein
   a. the mGnP chemotherapy consists of administering nab-paclitaxel and gemcitabine to the patient in need thereof only on days 1 and 15 of a 28-day treatment cycle; and
   b. the mFFX chemotherapy consists of administering to the patient in need thereof only on days 1 and 15 of a 28-day treatment cycle: 85 mg/m2 oxaliplatin to the patient in need thereof via IV infusion over 2 hours; in combination with administering 150 mg/m2 irinotecan via IV infusion over 90 min; and further administering 2,400 mg/m2 fluorouracil to the patient in need thereof over 46 hours beginning on Day 1 and ending on Day 3 of the 28-day treatment cycle.
14. The method of alternative 13, wherein the atebimetinib is administered in combination with the mGnP chemotherapy to treat the pancreatic cancer.
15. The method of alternative 14, wherein the pancreatic cancer has a KRAS mutation.
16. The method of alternative 14, wherein the mGnP chemotherapy consists of administering to the patient in need thereof only on days 1 and 15 of a 28-day treatment cycle: 125 mg/m2 nab-paclitaxel via IV infusion over 30 min to the patient in need thereof, followed immediately by, administering 1000 mg/m2 gemcitabine to the patient in need thereof via IV infusion over 30 min.
17. The method of alternative 14, wherein the atebimetinib is administered in combination with the mFFX chemotherapy to treat the pancreatic cancer.
18. The method of alternative 17, wherein the pancreatic cancer has a KRAS mutation.
19. The method of alternative 18, wherein the patient is treated with the mFFX chemotherapy consisting of administering to the patient in need thereof only on days 1 and 15 of a 28-day treatment cycle: 85 mg/m2 oxaliplatin to the patient in need thereof via IV infusion over 2 hours; in combination with administering 150 mg/m2 irinotecan via IV infusion over 90 min; and further administering 2,400 mg/m2 fluorouracil to the patient in need thereof over 46 hours beginning on Day 1 and ending on Day 3 of the 28-day treatment cycle.
20. The method of alternative 9, wherein the pancreatic cancer has a GNAS mutation.

In some alternatives, the following methods of treatment are provided:
21. A method of treating pancreatic cancer in a patient in need thereof, the method comprising administering Compound A

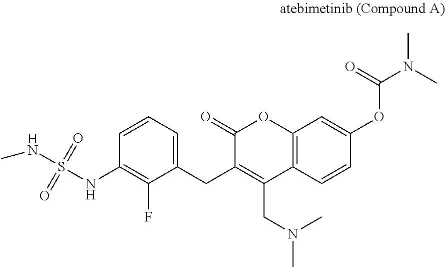

or a pharmaceutically acceptable salt thereof to the patient a dose providing 320 mg or 240 mg of Compound A once daily to the patient in need thereof.
22. The method of alternative 21, wherein a total of 320 mg of Compound A is orally administered (po) once daily (QD) to treat the cancer.
23. The method of any one of alternatives 21-22, wherein the cancer is pancreatic ductal adenocarcinoma (PDAC).
24. The method of any one of alternatives 21-23, wherein the cancer has a KRAS mutation.
25. The method of alternative 24, wherein the KRAS mutation is a G12C, G12D, G12A, G12R, G12S, G12V, Q61 or G13 mutation
26. The method of alternative 24, wherein the KRAS mutation is a G12D mutation.
27. The method of alternative 24, wherein the KRAS mutation is a G12V mutation.
28. The method of alternative 24, wherein the KRAS mutation is a G12C, G12V, G12D or G12R mutation.
29. The method of alternative 24, wherein the cancer optionally has a KRAS G12V, G12D or G12R mutation, and the method further comprises (a) treating the PDAC with one or more chemotherapy agents selected from the group consisting of gemcitabine, capecitabine, docetaxel and nab-paclitaxel, or (b) treating the PDAC with one or more chemotherapy agents selected from the group consisting of oxaliplatin, 5-fluorouracil and irinotecan.
30. A method of treating pancreatic ductal adenocarcinoma (PDAC) comprising the steps of administering to a patient in need thereof: oral administration of 240 mg of Compound A once per day, in combination with a modified gemcitabine+nab-paclitaxel (mGnP) regimen where the mGnP regimen is administered on Day 1 and Day 15 of each 28-day cycle as follows: nab-paclitaxel 125 mg/m2 via IV infusion over 30 minutes, followed immediately by gemcitabine 1000 mg/m2 via IV infusion over 30 minutes.
31. A method of treating pancreatic ductal adenocarcinoma (PDAC) comprising the steps of administering to a patient in need thereof: oral administration of 320 mg of Compound A once per day, in combination with a modified gemcitabine+nab-paclitaxel (mGnP) regimen where the mGnP regimen is administered on Day 1 and Day 15 of each 28-day cycle as follows: nab-paclitaxel 125 mg/m2 via IV infusion over 30 min, followed immediately by gemcitabine 1000 mg/m2 via IV infusion over 30 min.
32. A method of treating pancreatic ductal adenocarcinoma (PDAC) comprising the steps of administering to a patient in need thereof: oral administration of 240 mg of Compound A once per day, in combination with a modified FOLFIRINOX (mFFX) regimen where the mFFX regimen is administered on Day 1 and Day 15 of each 28-day cycle for up to 6 cycles, as follows: oxaliplatin 85 mg/m2 via IV infusion over 2 hours, folinic acid (leucovorin) 400 mg/m2 via IV infusion over 2 hours, irinotecan 150 mg/m2 via IV infusion over 90 minutes, and fluorouracil 2400 mg/m2 continuous IV infusion given over 46 hours beginning Day 1 and ending on Day 3.
33. A method of treating pancreatic ductal adenocarcinoma (PDAC) comprising the steps of administering to a patient in need thereof: oral administration of 320 mg of Compound A once per day, in combination with a modified FOLFINOX (mFFX) regimen where the mFFX regimen is administered on Day 1 and Day 15 of each 28-day cycle for up to 6 cycles, as follows: oxaliplatin 85 mg/m2 via IV infusion over 2 hours, folinic acid (leucovorin) 400 mg/m2 via IV infusion over 2 hours, irinotecan 150 mg/m2 via IV infusion over 90 minutes, and fluorouracil 2400 mg/m2 continuous IV infusion given over 46 hours beginning Day 1 and ending on Day 3.
34. The method of any one of alternatives 21-33, wherein the Compound A is administered in an oral dosage form comprising Compound A

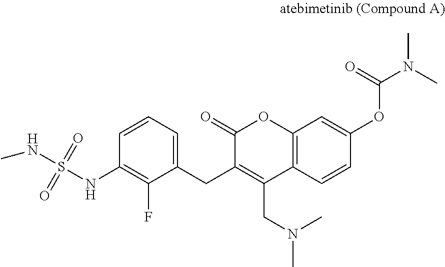

or a pharmaceutically acceptable salt and a pharmaceutically acceptable excipients to form an oral unit dosage form.
35. The method of alternative 34, wherein the pharmaceutically acceptable excipients comprises
an intra granulation excipients comprising a microcrystalline cellulose, a crospovidone, a hydroxypropyl cellulose; and
an extra granulation excipient comprising Compound A.
36. The method of any one of alternatives 34-35, wherein the unit dosage form comprises a total of 120 mg or 160 mg of Compound A.
37. The method of alternative 16, wherein the unit dosage form is a tablet.
38. The method of any one of alternatives 21-37, wherein the Compound A is administered in a second or later line of therapy.
39. The method of any one of alternatives 21-37, wherein the Compound A is administered as a first line of therapy.
40. The method of any one of alternatives 21-39, wherein the Compound A is administered once daily for at least 28 consecutive days.
41. A method of treating pancreatic cancer having a KRAS G12 mutation, the method comprising administering a therapeutically effective amount of atebimetinib or a pharmaceutically acceptable salt thereof to a patient in need thereof in combination with gemcitabine and nab-paclitaxel.
42. A method of treating pancreatic cancer, the method comprising administering a therapeutically effective amount of atebimetinib or a pharmaceutically acceptable salt thereof to a patient in need thereof in combination with gemcitabine and nab-paclitaxel.
43. A method of treating pancreatic cancer having a KRAS G12 mutation, the method comprising administering a therapeutically effective amount of atebimetinib or a pharmaceutically acceptable salt thereof to a patient in need thereof in combination with irinotecan and fluorouracil, and optionally leucovorin.
44. A method of treating pancreatic cancer, the method comprising administering a therapeutically effective amount of atebimetinib or a pharmaceutically acceptable salt thereof to a patient in need thereof in combination with irinotecan and fluorouracil.
45. A method of treating pancreatic cancer having a KRAS G12 mutation, the method comprising administering a therapeutically effective amount of atebimetinib or a pharmaceutically acceptable salt thereof to a patient in need thereof in combination with irinotecan, fluorouracil and oxaliplatin, and optionally leucovorin.
46. A method of treating pancreatic cancer, the method comprising administering a therapeutically effective amount of atebimetinib or a pharmaceutically acceptable salt thereof to a patient in need thereof in combination with irinotecan, fluorouracil and oxaliplatin, and optionally leucovorin.
47. A method of treating pancreatic cancer having a KRAS G12 mutation, the method comprising administering a therapeutically effective amount of atebimetinib or a pharmaceutically acceptable salt thereof to a patient in need thereof in combination with irinotecan, fluorouracil and a platinum based chemotherapy based chemotherapy agent, and optionally leucovorin.
48. A method of treating pancreatic cancer, the method comprising administering a therapeutically effective amount of atebimetinib or a pharmaceutically acceptable salt thereof to a patient in need thereof in combination with irinotecan, fluorouracil and a platinum based chemotherapy based chemotherapy agent, and optionally leucovorin.
49. The method of any one of alternatives 41-48, wherein the therapeutically effective amount of atebimetinib is a dose providing 320 mg QD to the patient in need thereof.
50. The method of alternative 49, wherein the atebimetinib is orally administered.
51. The method of any one of alternatives 49-50, wherein
the mGnP chemotherapy consists of administering to the patient in need thereof only on days 1 and 15 of a 28-day treatment cycle: 125 mg/m2 nab-paclitaxel to the patient in need thereof, and administering 1000 mg/m2 gemcitabine to the patient in need thereof; and
the mFFX chemotherapy consists of administering to the patient in need thereof only on days 1 and 15 of a 28-day treatment cycle: 85 mg/m2 oxaliplatin to the patient in need thereof via IV infusion over 2 hours; in combination with administering 150 mg/m2 irinotecan via IV infusion over 90 min; and further administering 2,400 mg/m2 fluorouracil to the patient in need thereof over 46 hours beginning on Day 1 and ending on Day 3 of the 28-day treatment cycle.
54. The method of alternative 51, wherein
the mGnP chemotherapy consists of administering to the patient in need thereof only on days 1 and 15 of a 28-day treatment cycle: 125 mg/m2 nab-paclitaxel via IV infusion over 30 min to the patient in need thereof, followed immediately by, administering 1000 mg/m2 gemcitabine to the patient in need thereof via IV infusion over 30 min; and
the mFFX chemotherapy consists of administering to the patient in need thereof only on days 1 and 15 of a 28-day treatment cycle: 85 mg/m2 oxaliplatin to the patient in need thereof via IV infusion over 2 hours; in combination with administering 150 mg/m2 irinotecan via IV infusion over 90 min; and further administering 2,400 mg/m2 fluorouracil to the patient in need thereof over 46 hours beginning on Day 1 and ending on Day 3 of the 28-day treatment cycle.
57. The method of alternative 51 or 52, wherein the atebimetinib is administered in combination with the mGnP chemotherapy.
58. The method of any one of alternatives 51-53, wherein the atebimetinib is administered in combination with the mFFX chemotherapy.
59. The method of alternative 51 or 52, wherein the atebimetinib is administered in combination with the mGnP chemotherapy without the mFFX chemotherapy.
60. The method of alternative 51 or 52, wherein the atebimetinib is administered in combination with the mFFX chemotherapy without the mGnP chemotherapy.
61. The method of any one of alternatives 51-56, wherein the method is administered to the patient in need thereof for one or more 28-day treatment cycles, to treat the pancreatic cancer.
62. The method of any one of alternatives 51-57, wherein the method is administered to the patient as a first line treatment of the pancreatic cancer.
63. The method of any one of alternatives 51-57, wherein the method is administered to the patient as a second line treatment of the pancreatic cancer.
64. The method of alternative 59, wherein the atebimetinib is administered in combination with the mGnP chemotherapy but not the mFFX chemotherapy.
65. The method of alternative 59, wherein the atebimetinib is administered in combination with the mFFX chemotherapy but not the mGnP chemotherapy.
66. The method of any one of alternatives 59-61, wherein the atebimetinib is administered in combination after the pancreatic cancer was previously treated with a first line chemotherapy without atebimetinib.
67. The method of alternative 62, wherein the first line chemotherapy comprises one or more chemotherapy agents selected from the group consisting of irinotecan, fluorouracil and a platinum-based chemotherapy agent.
68. The method of alternative 62, wherein the first line chemotherapy comprises one or more chemotherapy agents selected from the group consisting of gemcitabine and nab-paclitaxel.
69. The method of any one of alternatives 51-57, wherein the method is administered to the patient as a third or subsequent line treatment of the pancreatic cancer.
70. The method of alternative 65, wherein the atebimetinib is administered in combination with the mGnP chemotherapy but not the mFFX chemotherapy.
71. The method of alternative 65, wherein the atebimetinib is administered in combination with the mFFX chemotherapy but not the mGnP chemotherapy.
72. The method of any one of alternatives 65-67, wherein the atebimetinib is administered in combination after the pancreatic cancer was previously treated with a first or subsequent line chemotherapy without atebimetinib.
73. The method of alternative 68, wherein the first line chemotherapy comprises one or more chemotherapy agents selected from the group consisting of irinotecan, fluorouracil and a platinum-based chemotherapy agent.
74. The method of alternative 68, wherein the first line chemotherapy comprises one or more chemotherapy agents selected from the group consisting of gemcitabine and nab-paclitaxel.

### EXAMPLES

Additional embodiments are disclosed in further detail in the following representative examples, which are not in any way intended to limit the scope of the claims. The following examples are provided for the guidance of the reader, and represent certain methods for making and using the compounds and compositions exemplified herein. These methods are not limiting, and it will be apparent that other routes may be employed to prepare these compounds. The compound numberings used in the synthetic schemes depicted below are meant for those specific schemes only, and should not be construed as or confused with same numberings in other sections of the application.

### Example 1

### Synthesis of Compound A

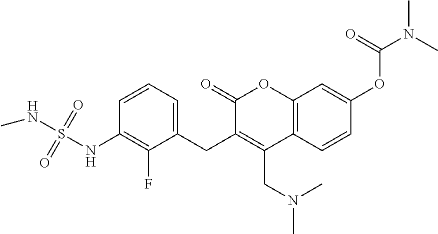

Compound A was prepared in 1 step: 4-(bromomethyl)-3-(2-fluoro-3-((N-methylsulfamoyl)amino)benzyl)-2-oxo-2H-chromen-7-yl dimethylcarbamate (22.22 g, 34.79 mmol) was suspended in methanol. Dimethylamine 2M was added and the formed reaction mixture was stirred until full conversion was observed. After full conversion the reaction was concentrated under reduced pressure. 1M HCl was added to the residue and the water layer was extracted with CH2Cl2. The water layer was made basic with solid Na2CO3. The basic water layer was extracted with CH2Cl2. The organic layer from the basic extraction was washed with brine, dried over Na2SO4, filtered and concentrated under reduced pressure to obtain the title compound (13.23 g, 25.7 mmol, yield: 74%) as a light yellow solid.

Yield: Compound A was isolated as a light yellow solid (74% over 1 step). Analysis: LCMS (Method T): t_{R}=1.53 min; m/z calculated for [M−H]⁺=507.2, found=507.2; ¹H NMR (400 MHz, DMSO) δ 9.38 (s, 1H), 8.08 (d, J=8.8 Hz, 1H), 7.28 (td, J=8.0, 1.6 Hz, 1H), 7.25-7.18 (m, 2H), 7.15 (dd, J=8.8, 2.4 Hz, 1H), 7.00 (t, J=7.9 Hz, 1H), 6.90-6.77 (m, 1H), 4.04 (s, 2H), 3.64 (s, 2H), 3.06 (s, 3H), 2.93 (s, 3H), 2.52 (d, J=4.9 Hz, 3H), 2.19 (s, 6H).

### Example 2

### Compound A Nonclinical Pharmacology

Compound A is a potent, non-adenosine 5′-Triphosphate (ATP) competitive selective dual MEK inhibitor with the biochemical inhibitory half-maximal inhibitory concentration (IC₅₀) for activated ERK of 24.2 nM to 83.2 nM across several cancer disease types. Compound A has been shown to have in vivo anti-tumor activity in mouse syngeneic and human xenograft models of activating MAPK pathway mutations in KRAS, NRAS, HRAS, BRAF, neurofibromatosis type 1 (NF1), and KSR, with the most sensitive being predominantly pancreatic cancer cell lines that harbor activating MAPK pathway mutations in KRAS, NRAS, HRAS, BRAF, NF1, and KSR (Table 5).

TABLE 5
(IC50 values for pERK in Multiple 2D Grown Cell Lines)

| Tumor cell line and | Key MAPK Activating | |
|---|---|---|
| MAPK mutation | Mutation(s) | IC₅₀ nM^{a} |
| SK-MEL-2 NRAS^{Q6IR} | NRAS: p.Q61R, | 26.19 to |
| Human pancreatic | BRAF: Amplified, | 36.17 |
| adenocarcinoma | RAF1: Amplifeid, | |
| | MAP2K2: Amplified | |
| MIA PaCa-2 KRAS^{G12C} | KRAS: p.G12C, | 49.39 |
| Human pancreatic | NRAS: Amplified, | |
| adenocarcinoma | BRAF: Amplified, | |
| | MAP2K2: Amplified, | |
| | KSR1: Amplified | |

2D = two dimensional;
Amp = amplified;
BRAF = B isomer of rapidly accelerated fibrosarcoma (RAF);
IC50 = 50% inhibitory concentration;
KRAS = K isomer of rat sarcoma (RAS);
KSR = kinase suppressor of Ras;
MAP2K2 = mitogen-activated protein kinase kinase 2;
MEL = melanoma;
NF1 = neurofibromatosis type 1;
NRAS = N isomer of rat sarcoma (RAS);
RAF1 = Raf-1 proto-oncogene, serine/threonine kinase;
SOS1 = son of sevenless 1
^{a}Potency observed in cell-based assays (range shown when experiment was repeated)

Corresponding reductions in phosphorylated MEK (pMEK) were observed within 2-hours following treatment of cells with 100 nM Compound A, confirming that Compound A was not sensitive to the MAPK pathway feedback reactivation events, such as CRAF-bypass, that are a known limitation of first generation MEK inhibitors currently in clinical use.

### Example 3A

### Preclinical Studies: Compound A in MIA PaCa-2 (KRAS G12C) and Capan-2 (KRAS-G12V) Pancreatic Cancer Xenograft Tumor Models

Compound A was evaluated head-to-head against sotorasib (AMG-510) and gemcitabine alone, and Compound A in combination with sotorasib, for 21 days in the KRAS G12C xenograft model (i.e., MIA PaCa-2 (KRAS) Pancreatic Xenograft Tumor Model in Athymic Nude Mice). Compound A as compared to sotorasib demonstrated tumor regression, both with insignificant BWL. Comparing Compound A alone, against sotorasib and in combination with sotorasib, tumor regressions with insignificant median BWL were observed (i.e., within 3% of baseline), which indicates activity, durability and tolerability of Compound A against a KRAS G12C mutant pancreatic cancer model. FIG. 2A shows data from the head-to-head Comparison of Compound A+/−sotorasib (AMG-510) using a MIA PaCa-2 Xenograft Tumor Model: Tumor Volume. Compound A was evaluated head-to-head against sotorasib (AMG-510) and adagrasib alone for 21 days in the Capan-2 (KRAS G12V) Pancreatic Xenograft Tumor Model in Athymic Nude Mice. As shown in FIG. 2B, Compound A demonstrated tumor regression as compared to sotorasib or adagrasib, with insignificant BWL. In FIGS. 2A and 2B: Tumor Growth Inhibition (TGI) %=[1−(Ti−T₀)/(Ci−C₀)]×100; Expanded TGI formula vs. previous 1−[T/C]×100% method.

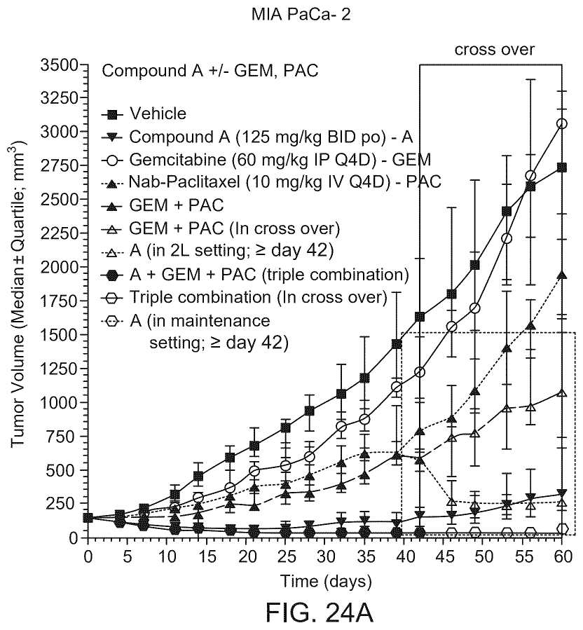

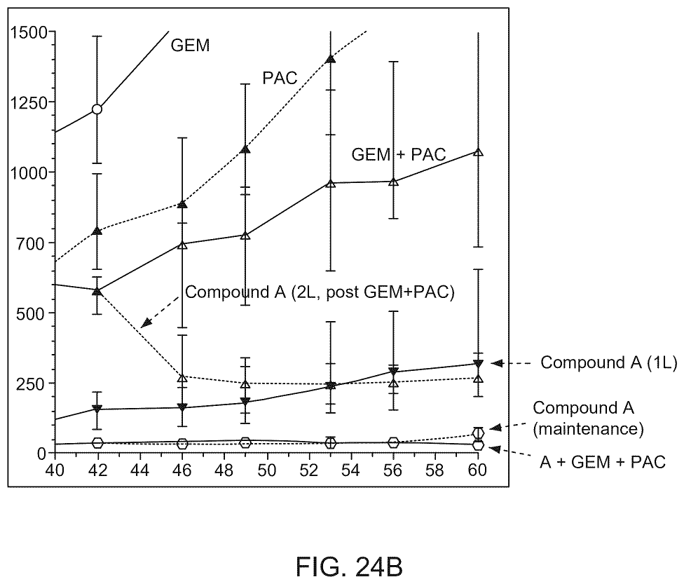

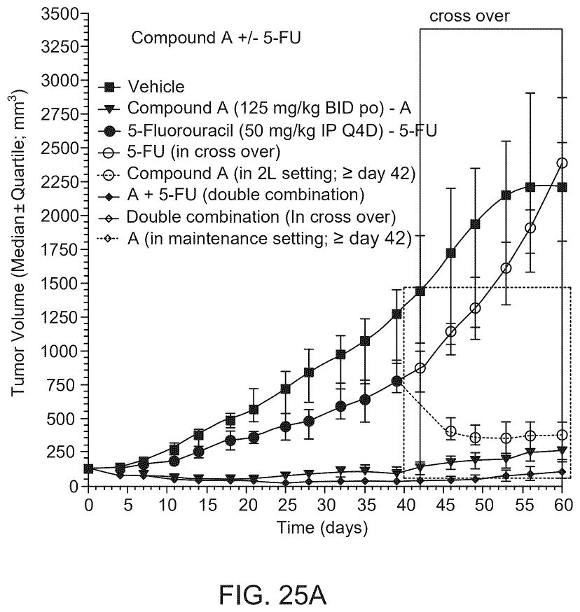

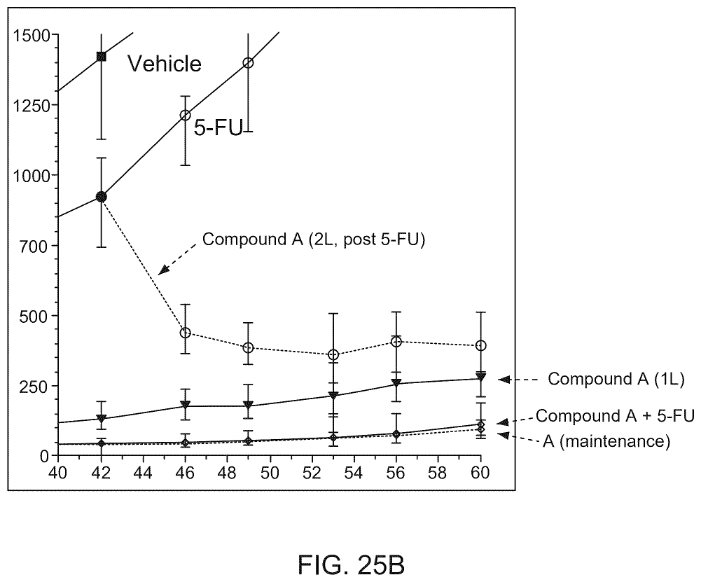

Compound A was evaluated in a MIA PaCa-2 Pancreatic (KRAS G12C) xenograft tumor models in athymic nude mice. Gemcitabine (antimetabolite), nab-paclitaxel (taxane) and fluorouracil (5-FU; antimetabolite) were commercially purchased. All studies started with 12 animals, per group. Mice within specific groups randomized for cross-over of treatments (≥day 42). Compound A+GEM (doublet combination) similar but slightly inferior to triple combination (A+GEM+PAC). FIG. 24A is a graph showing data obtained from an evaluation of Compound A (A), gemcitabine (GEM) or nab-paclitaxel (PAC) individually or in the specified combinations, as compared to vehicle using a MIA PaCa-2 KRAS G12C mutant xenograft tumor model in athymic nude mice (Tumor Volume). FIG. 24B is a graph showing a magnified portion of the graph in FIG. 24A, showing data obtained from an evaluation of Compound A (A), gemcitabine (GEM) or nab-paclitaxel (PAC) individually or in the specified combinations, as compared to vehicle using a MIA PaCa-2 KRAS G12C mutant xenograft tumor model in athymic nude mice (Tumor Volume). FIG. 25A is a graph showing data obtained from an evaluation of Compound A (A), and 5-fluorouracil (5-FU) individually or in combination, as compared to vehicle using a MIA PaCa-2 KRAS G12C mutant xenograft tumor model in athymic nude mice (Tumor Volume). FIG. 25B is a graph showing an magnified portion of the graph in FIG. 25A, showing data obtained from an evaluation of Compound A (A), and 5-fluorouracil (5-FU) individually or in combination, as compared to vehicle using a MIA PaCa-2 KRAS G12C mutant xenograft tumor model in athymic nude mice (Tumor Volume). The Bliss Combination Index (CI) and Synergy Score (SS) of Compound A was calculated in combination in vivo from above MIA PaCa-2 Tumor Xenograft Efficacy studies. The results are shown in Table 6A (Compound A in combination with 5-FU) and Table 6B (Compound A in combination with nab-paclitaxel and gemcitabine). The Twelve pancreatic tumor models were evaluated (3D-TGA model) in combinations of Compound A with either gemcitabine, paclitaxel or 5-FU. 10 of 12 (83%) of the models showed additivity as assessed by ZIP synergy scores between −10 and +10 for each treatment. Additive models: MIA PaCa-2, PA-TU-89885, CFPAC-1, HPAF-II, Panc 03.27, PSN-1, Panc 10.05, Capan-1, HPAC, PANC-1. Capan-2 and AsPC-1 failed to reach additive cutoffs for 5-FU alone.

TABLE 6A

| | AUC Combination | | AUC Synergy | | | |
|---|---|---|---|---|---|---|
| | Index (CI) +/− | | Score (SS) +/− | | | |
| | 95% Confid. Int. | | 95% Confid. Int. | | Synergy Call | |
| Comparison | Day 39 | Day 60 | Day 39 | Day 60 | Day 29 | Day 60 |
| 75 mg/kg PO | 0.30 | 0.34 | 8.60 | 2.50 | Synergistic | Additive |
| BID Compound | [0.17-0.51] | [0.07-1.40] | [3.7-16.0] | [−1.00-6.40] | | |
| A + | | | | | | |
| 50 mg/kg IP Q4D | | | | | | |
| 5-FU | | | | | | |
| 125 mg/kg PO | 0.30 | 0.47 | 3.00 | 0.41 | Synergistic | Additive |
| BID Compound | [0.20-0.46] | [0.17-1.20] | [1.30-5.90] | [−0.09-1.20] | | |
| A + | | | | | | |
| 50 mg/kg IP Q4D | | | | | | |
| 5-FU | | | | | | |

TABLE 6B

| | AUC Combination | | AUC Synergy | | | |
|---|---|---|---|---|---|---|
| | Index (CI) +/− | | Score (SS) +/− | | | |
| | 95% Confid. Int. | | 95% Confid. Int. | | Synergy Call | |
| Comparison | Day 39 | Day 60 | Day 39 | Day 60 | Day 29 | Day 60 |
| 60 mg/kg IP | 0.72 | 0.53 | 10.0 | 9.9 | A | A |
| Q4D GEM + | [0.43-1.10] | [0.16-1.30] | [−3.50-26.0] | [−4.60-26.0] | | |
| 10 mg/kg IV | | | | | | |
| Q4D PAC | | | | | | |
| 75 mg/kg PO | 0.21 | 0.28 | 12.0 | 4.00 | S | S |
| QD Cmpd A + | [0.14-0.30] | [0.14-0.57] | [7.30-19.0] | [1.40-8.50] | | |
| 60 mg/kg IP | | | | | | |
| Q4D GEM | | | | | | |
| 125 mg/kg PO | 0.18 | 0.12 | 4.50 | 1.00 | S | S |
| QD Cmpd A + | [0.12-0.27] | [0.042-0.36] | [2.60-7.70] | [0.39-2.10] | | |
| 60 mg/kg IP | | | | | | |
| Q4D GEM | | | | | | |
| 75 mg/kg PO | 0.23 | 0.22 | 4.40 | 0.83 | S | S |
| QD Cmpd A + | [0.14-0.39] | [0.06-0.96] | [2.00-8.40] | [0.02-2.80] | | |
| 60 mg/kg IP | | | | | | |
| Q4D GEM + | | | | | | |
| 10 mg/kg IV | | | | | | |
| Q4D PAC | | | | | | |
| 125 mg/kg PO | 0.41 | 0.83 | 1.20 | 0.04 | S | A |
| QD Cmpd A + | [0.21-0.77] | [0.21-3.60] | [0.28-2.90] | [−0.21-0.47] | | |
| 60 mg/kg IP | | | | | | |
| Q4D GEM + | | | | | | |
| 10 mg/kg IV | | | | | | |
| Q4D PAC | | | | | | |

GEM = gemcitabine;
PAC = nab-paclitaxel;
Cmpd A = Compound A
S = Synergistic;
A = Additive

### Example 3B

### Treatment-Acquired Mutations in MIA PaCa-2 (KRAS G12C) Pancreatic Cancer Xenograft Tumor Models

An analysis of treatment-acquired mutations in was performed in the mice treated with Compound A, 5-fluorouracil, gemcitabine and nab-paclitaxel. DNA analysis and mutational enrichment analysis was performed. Tumor extracted from xenograft-induced mice was run through DNA isolation, library preparation, and Illumina exome sequencing via Crown Bio as previously described. Then, a bioinformatics analysis was performed.

For whole exome sequencing data processing, the following workflow was implemented on DNAnexus. First, separation of reads into human and mouse sequence was conducted BBSplit (Bushnell B), where human reads were retained for subsequent analysis. Next, adaptor trimming was performed via BBDuk (Bushnell B). The adaptor-cleaved reads were then assessed via quality control method FastQC. Alignment was conducted on the adaptor-trimmed reads via BWA (Li 2013). Duplicate marking and base quality score recalibration was achieved via GATK (Poplin 2017), and single-sample somatic variant calling via MuTect2 (GATK package and using both a provided panel of normal samples and gnomAD from the GATK resource bundle) following the best-practices workflow. CNVkit (Talevich 2016) was used in a tumor-only fashion for copy-number calling.

For tertiary analysis of the mutation data, the resultant VCFs generated via MuTect2 were processed via the VCF2MAF pipeline (Kandoth 2020). The resultant MAF files were processed such that variants with a filter status of “PASS”, “clustered_events”, and “haplotype” were accepted. For mutation evaluation, if the mutation occurred in BRAF, it was compared to a literature-assessed list for classification into known Class 1, Class 2, or Class 3 mutations, it was considered damaging; if the mutation was in RAF1, nonsynonymous, and the affected locus was in either amino acids S257 or S2579, it was considered damaging; if the mutation was in ARAF and was a nonsynonymous mutation of S214, it was considered damaging; if the mutation was in KRAS, HRAS, or NRAS and a nonsynonymous substitution occurred in a known pathogenic residue (G12, G13, A59, G60, Q61, K117, A146), it was considered damaging. For all other genes, a mutation was considered damaging if it was in a known cancer hotspot, was annotated containing the description deleterious via SIFT, high impact variant consequence from Ensembl, present in a pfam or PANTHER domain, or was annotated containing the description “pathogenic” via CLIN_SIG (clinically significant annotation from dbSNP). Mutations were filtered by presence in at least one public patient dataset (GENIE, TCGA) and at least one public cell line dataset (CCLE, DepMap, SCMP).

A computational biology assessment was performed. Mutations were categorized by treatment class. Mice left untreated or treated with a vehicle served as the background group. Specific point mutations identified in these mice were excluded from subsequent analysis. To identify selection pressure, the remaining mutations were tallied, ensuring each mutated gene was counted only once per mouse (e.g., mutations in the same gene, YFG, such as point mutations c.A123C and c.T124C from a single mouse, were counted only once). The effect of drug selection was determined by tallying the frequency of gene mutations within each treatment group. The treatment groups were as follows:
Compound A: 75 treated with Compound A and 125 treated with Compound A
Gemcitabine (GEM): 60 treated with Gemcitabine and 60 treated with Gemcitabine+10 Nab-paclitaxel
Nab-paclitaxel (PAC): 10 treated with Nab-paclitaxel and 10 treated with Nab-paclitaxel+60 Gemcitabine
5-fluorouracil (5-FU): 50 treated with 5-Fluorouracil
Compound A+GEM+PAC: 75 treated with Compound A+60 Gemcitabine+10 Nab-paclitaxel and 125 treated with Compound A+60 Gemcitabine+10 Nab-paclitaxel
Compound A+5FU: 75 treated with Compound A+50 5-FU and 125 treated with Compound A+50 5-FU

To identify the most frequently mutated genes, thresholds were applied to each treatment group to capture mutations in at least 65 genes per group. These genes were further analyzed using Enrichr (Xie 2021), employing the Transcriptional Protein Hubs gene set. Volcano plots were generated, with the y-axis representing −log 10 (adjusted p-value) and the x-axis representing the odds ratio.

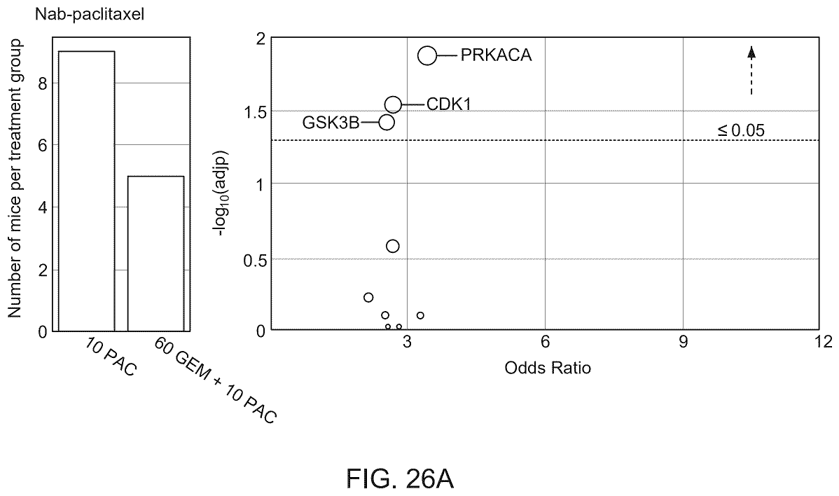

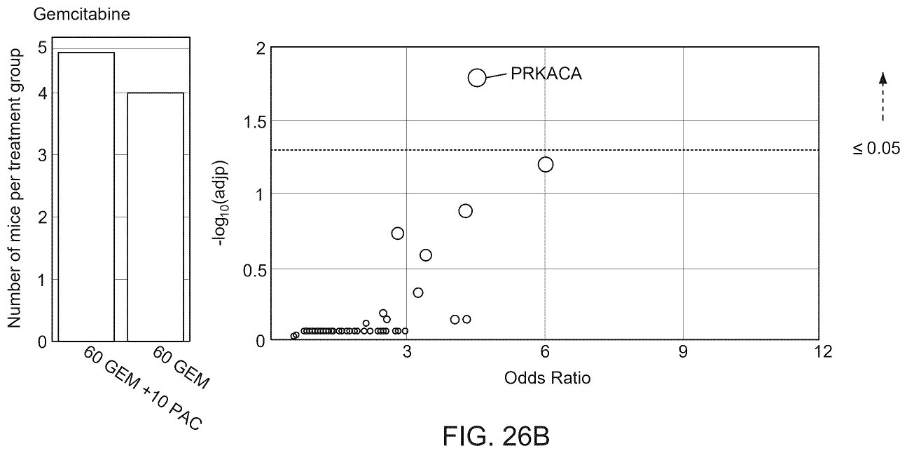

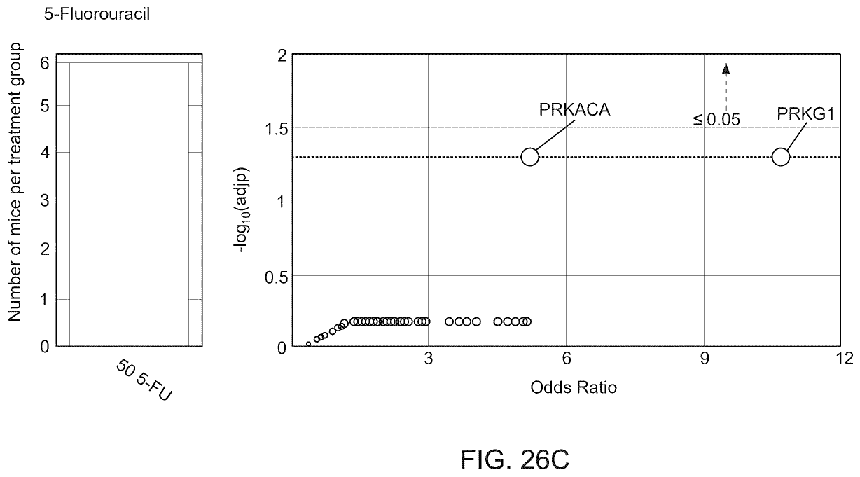

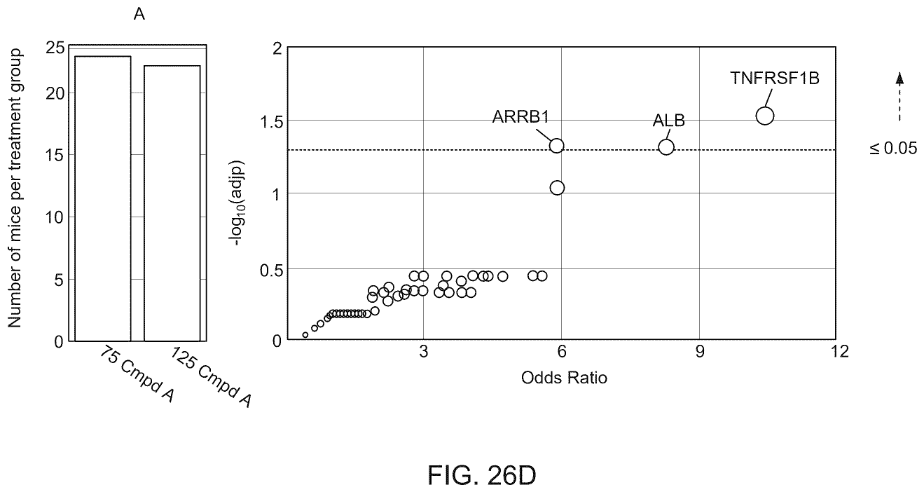

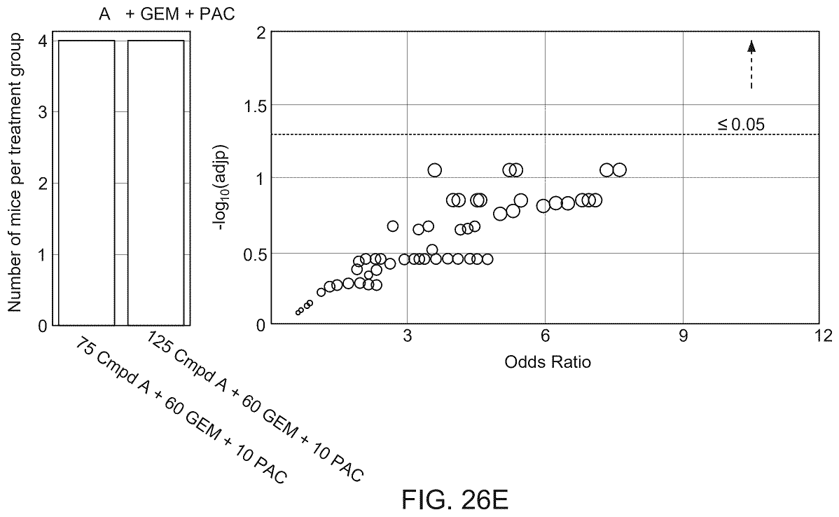

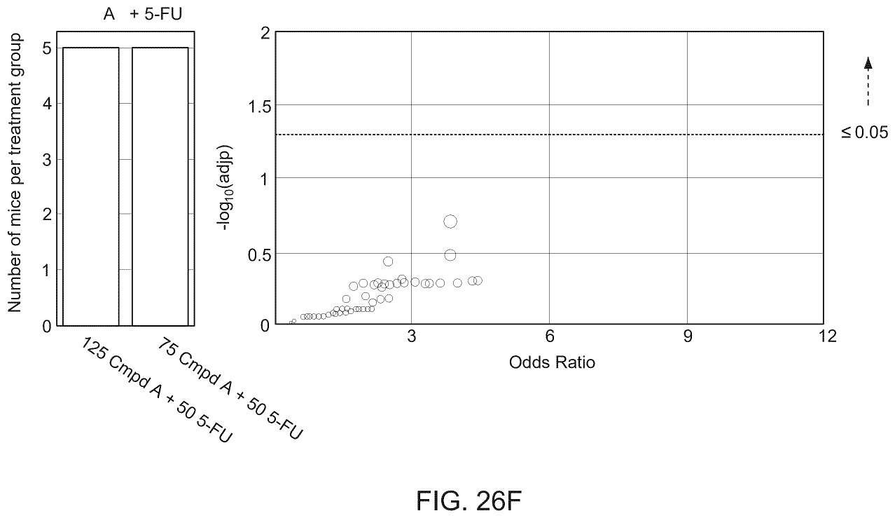

Results are shown in FIGS. 26A-26F. FIGS. 26A-F are graphs showing the results form an enrichment analysis of recurrently-mutated genes under continuous treatment in protein-protein interaction hubs for nab-paclitaxel (FIG. 26A), gemcitabine (FIG. 26B), 5-fluorouracil (FIG. 26C), Compound A (FIG. 26D), a combination of Compound A, gemcitabine and nab-paclitaxel (FIG. 26E) and a combination of Compound A and 5-fluorouracil (FIG. 26F), showing the number of mice per treatment group and a scatter plot of the corresponding odds ratio. These data show that PRKACA activates MAPK signaling via RAF; with 5-FU treatment or combination therapy using Nab-paclitaxel+Gemcitabine, tumors exhibit increased reliance on MAPK for survival and proliferation. Notably, this enrichment is absent in Compound A monotherapy or combinations. CDK1 mediates paclitaxel resistance, while GSK3B acts as a compensatory mechanism in paclitaxel treatment; co-treatment with a GSK3 inhibitor enhances paclitaxel efficacy. PRKG1 gene set itself is likely not a driver of resistance, but its component gene BRAF signals through MAPK. TNFRSF1B regulates PI3K-Akt, a MAPK-independent pro-survival pathway. ARRB1 facilitates ERK auto-phosphorylation in the absence of MEK.7

While pancreatic tumors display near universal activation of the RAS/MAPK pathway, not all tumors are addicted to this pathway alone. A goal of combination therapy is to drive deeper, more durable antitumor responses, and Compound A showed promising combination effects with GEM, PAC and 5-FU in multiple 3D-TGA pancreatic cancer models. Further, Compound A alone showed greater tumor growth inhibition (TGI) head-to-head versus single or combination chemotherapy in MIA PaCa-2. Combinations of Compound A plus standard-of-care chemotherapies demonstrated synergy in vivo and resulted in near complete responses in a majority of animals.

The ongoing Phase 2a clinical trial includes five arms, three of which focus on pancreatic cancer, where Compound A is being evaluated as both monotherapy and in combination with approved GEM and 5-FU containing chemotherapy combinations. These new in vitro, in vivo and ML modeling data further support an advancing translational roadmap for Compound A in pancreatic cancer.

### Example 4

### Humanized 3D Tumor Models

The humanized 3D tumor growth assay (3D-TGA) is an extracellular matrix (ECM)-based, 3D tumor model that more faithfully mimics the human tumor microenvironment (TME) with the goal of improving translational fidelity to patients. See Casneuf T, et al., “Interleukin-6 is a potential therapeutic target in interleukin-6 dependent, estrogen receptor-α-positive breast cancer,” Breast Cancer (Dove Med Press). 2016; 8:13-27; Onion D, et al. “3-Dimensional Patient-Derived Lung Cancer Assays Reveal Resistance to Standards-of-Care Promoted by Stromal Cells but Sensitivity to Histone Deacetylase Inhibitors,” Mol Cancer Ther. 2016 April; 15(4):753-63; and Sasser A K, et al., “Interleukin-6 is a potent growth factor for ER-alpha-positive human breast cancer,” FASEB J. 2007 November; 21(13):3763-70, each incorporated herein by reference in their entirety. Disruption of the MAPK pathway has specifically demonstrated that 3D models more accurately predict certain aspects of in vivo tumor biology, especially when evaluating molecular activities of drug candidates that target the MAPK pathway (See Janes M R, et al., “Targeting KRAS Mutant Cancers with a Covalent G12C-Specific Inhibitor,” Cell, 2018 Jan. 25; 172(3):578-589.e17, incorporated herein by reference in its entirety). MEK inhibition in the 3D-TGA. MEK inhibition in the 3D-TGA is useful to compare tumor addiction levels to the MAPK pathway across many models and establish response biomarkers for Compound A.

Novel dual-MEK inhibitor Compound A is under clinical investigation for use in RAS-addicted solid tumors. Approved KRAS G12C inhibitors are available but cover a limited subset of patients. For example, the KRAS G12C substitution occurs in only 1-3% of pancreatic cancers. The response to Compound A was assessed across RAS mutant preclinical models to determine whether a preference was observed for mutation position, or for specific amino acid substitutions.

Response to Compound A was measured in a humanized 3D tumor growth assay across 132 tumor models. A subset (N=107) of the 132 models were “RAS Mutant” (including H/K/N isoforms). Seventy-five of these models have a reported RAS mutation, and all models are being mutationally profiled by whole exome sequencing, with the majority (˜85%) completed to date. The RAS-mutant panel spans 12 tissue types and includes a subset of 30 confirmed KRAS G12 mutated cell lines drawn from three major indications: 12 pancreatic, 11 lung, and 7 colorectal cancer models. Based on the 3D assay, cell lines were classified into sensitive (IC50 less than 1 micromolar), intermediate (IC50 of at least 1 micromolar and greater than 25% reduction), and otherwise resistant to Compound A. The distribution of responses was then assessed across mutation position and amino acid substitutions. MAPK Normal additionally excluded models with BRAF (class I/II) and GNAQ/GNA11 mutations. Patient-aligned tumor models represent models where the mutational profile mapped to the most frequent 95% of GENIE v 13.0 patients of the same indication.

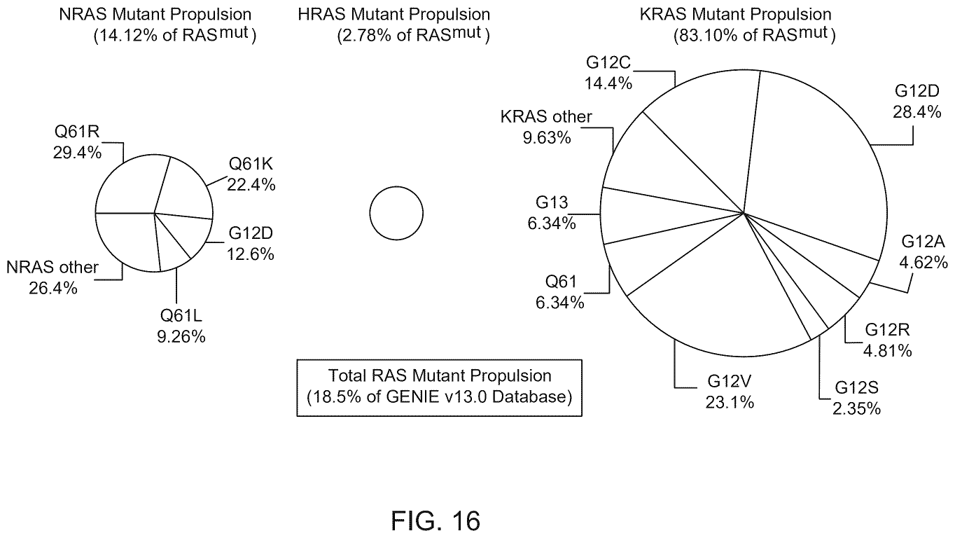

Across all RAS-mutant models, at least one model displayed response to Compound A for each observed mutation in K/N/HRAS. That is, no particular mutation position or amino acid substitution was exclusively found to confer resistance to drug exposure. FIG. 16 shows graphs representing the distribution of RAS mutations across patients in the GENIE database for NRAS mutant population (about 14% of the RAS mutations observed), the HRAS mutant population (about 3% of the RAS mutations observed), and the KRAS mutant population (about 83% of the RAS mutations observed).

Association of response with amino acid identity was further evaluated in a subset of lines for the most frequently altered residue in KRAS, G12. A distribution of responses was observed for each amino acid substitution. The G12 substitutions having at least four cell lines each total across the three indications included G12C (8 lines), D (5 lines), R (4 lines), and V (11 lines). Across cell lines for each of these substitutions, multiple response categories were observed. In each case, half or more lines fell into the intermediate category with the rest falling into one or both of the other response categories. For example, out of the 8 KRAS G12C lines, 6 showed intermediate response, 1 showed resistance, and 1 showed sensitivity. Examining these distributions together, no significant statistical relationship was seen between the amino acid substitution and response categories by Fisher's exact test.

Across 75 RAS-mutated cell lines, each mutation position or amino acid substitution was associated with sensitive or intermediate response in at least one line. Looking at the most commonly mutated position in KRAS, across 28 KRAS G12 mutated cell lines from three cancer indications, no significant preference was observed with respect to response to Compound A for a particular amino acid at G12. These observations suggest potential relevance of Compound A to a broad RAS-driven patient population.

Compound A, with Universal-RAS activity through Deep Cyclic Inhibition (DCI) of MEK, was evaluated in humanized 3D preclinical tumor models displaying diverse MAPK pathway activation events. Based on drug sensitivity and resistance profiles, a biomarker signature for Compound A was developed in order to project potential therapeutic response of cancer patients found in the AACR Project GENIE database.

Humanized 3D preclinical models better predict in vivo tumor responses versus 2D culture and more accurately replicate biology of human tumors. Therefore, the antitumor activity of Compound A was evaluated in over 132 tumor models spanning 12 distinct tumor types in the humanized 3D tumor growth assay (3D-TGA). Cell-based whole exome sequencing (WES) readouts were combined with 3D-TGA results to build a pharmacogenomic response algorithm. When applied to the GENIE patient database, resultant tumor-specific response landscapes helped to inform an early Universal-RAS clinical trial design for Compound A.

Cell lines tested in 3D-TGA (N=132) were assigned response of sensitive (IC50 <1 μM), intermediate (IC50 ≥1 and >25% reduction at 10 μM), and resistant otherwise

The dark line on each plot represents the median of the individual curves; Dotted vertical lines match Cmax Compound A drug free-fractions attained in patients 3 and 4 at 160 mg QD p.o.

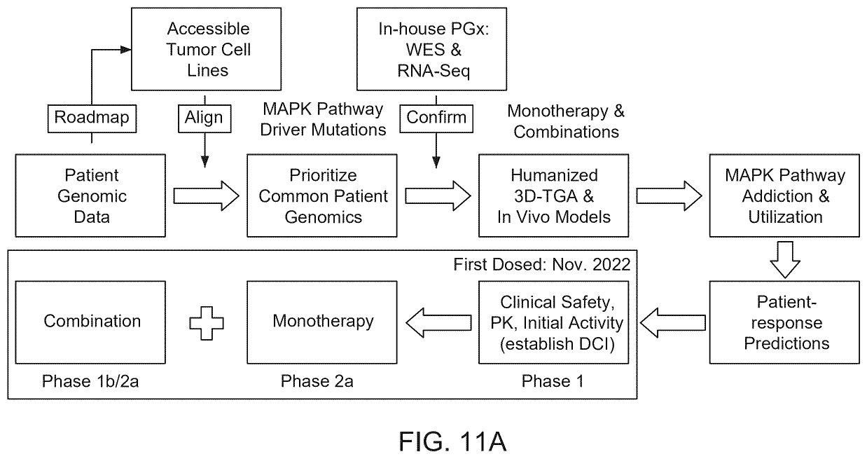

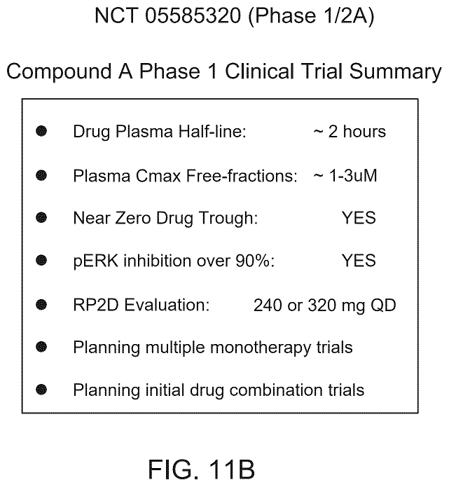

For 122 of the 3D-TGA cell lines where in-house WES data were also collected, the upper plateau-to-1 μM difference was used to delineate responder from non-responder, and thus train a logistic regression model with 10-fold cross validation. There were 28 features used, one comprised of a consolidated MAPK mutations status (mutations in K/H/NRAS, GNAQ/11, and BRAF class I/II), and 27 other genes representing key pathways. With an AUC of 0.84 on the training cohort and 0.72 on test cohort, the model was then applied to GENIE cohorts (see, FIGS. 11A-11C) where all features were included on the testing panel. For each indication of interest, a kernel density plot of the probability of predicted response for the entire cohort (projected onto the negative y-axis), and the MAPK(+) subset (positive y-axis) shows enrichment for higher probability of response with restriction to MAPK alteration status.

The depth of response to Compound A was evaluated across a panel of diverse 3D-TGA tumor models and led to identification of a biomarker signature for therapeutically addressable MAPK pathway addiction. To translate these findings into a relevant clinical application, a response algorithm was developed and applied to the GENIE database, which has cataloged the molecular profiles of over 100,000 cancer patients. Mutational landscapes of patients within GENIE helped identify preclinical models that better represent patient profiles likely to be encountered in the clinic. This approach could, as a general principle, be applied as a tool for improving biomarker discovery and clinical translation of oncology drugs. FIG. 3 shows mapping of biomarker sensitive patient populations (GENIE) (MAPK+=GENIE patients per indication with activation mutation in KRAS, NRAS, HRAS, BRAF (class I or II)).

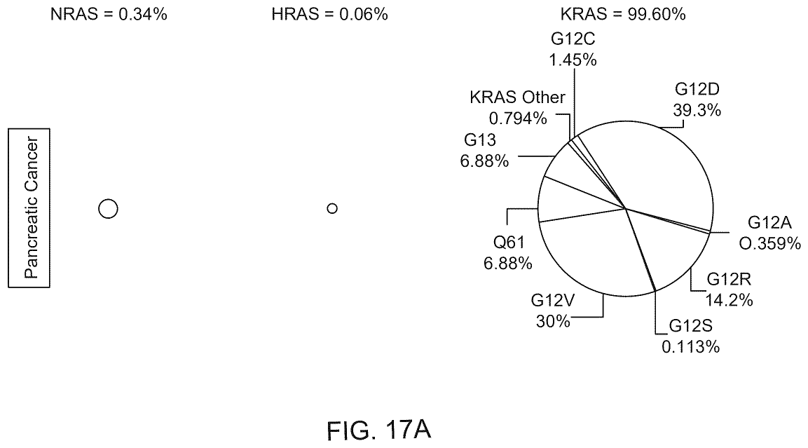

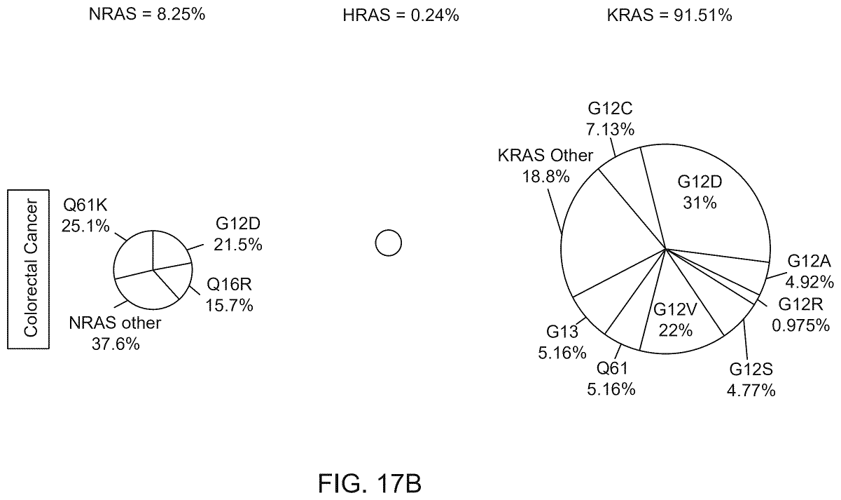

FIGS. 17A, and 17B are graphs showing the mutation profiles across indications in the GENIE patient database for pancreatic cancer cells with RAS mutations. FIG. 17A shows graphs of mutation profiles representing the distribution of RAS mutations across indications in the GENIE patient database for pancreatic cancer cells with NRAS (less than 1%), HRAS (less than 1%) and KRAS (greater than 99%). FIG. 17B shows graphs of mutation profiles representing the distribution of RAS mutations across indications in the GENIE patient database for melanoma cancer cells with NRAS (about 8%), HRAS (less than 1%) and KRAS (about 92%).

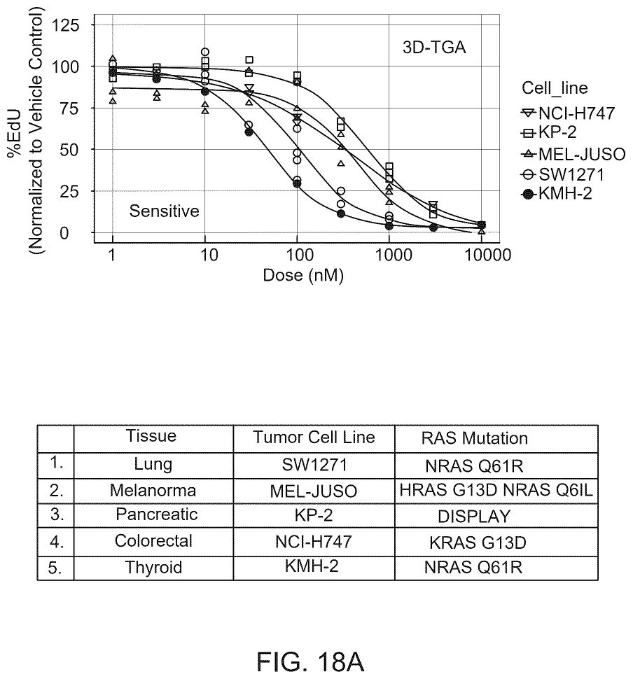

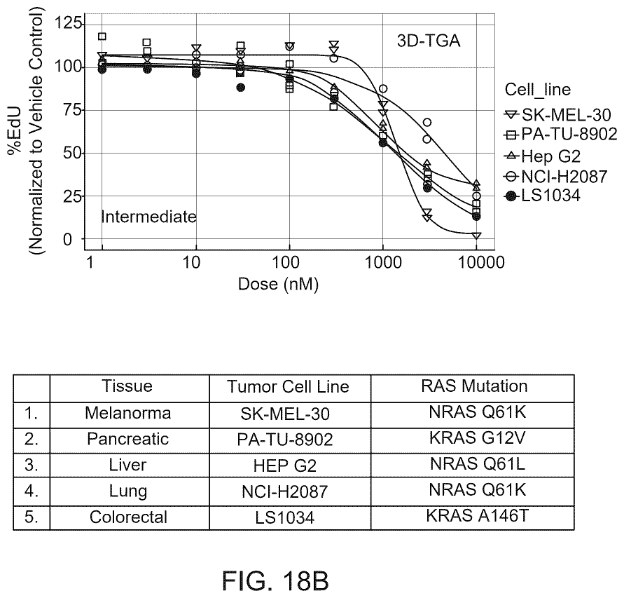

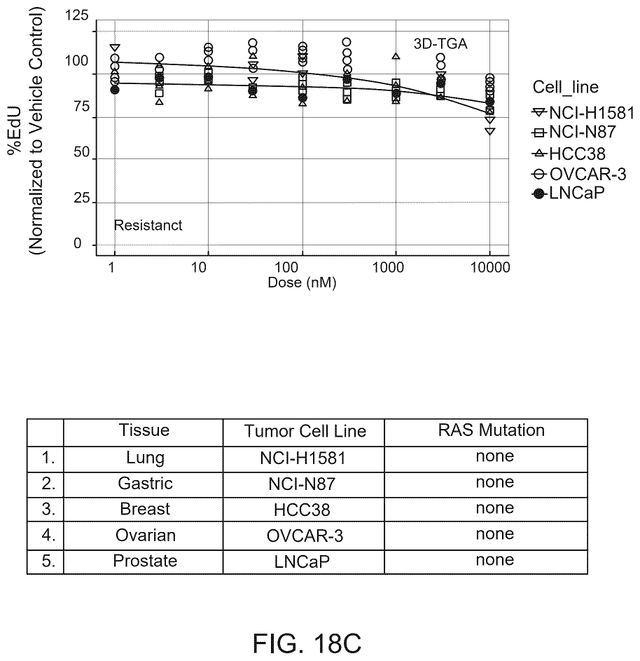

FIGS. 18A, 18B, and 18C show dose-response curves obtained with Compound A in the 3D-TGA model. Cell lines tested in 3D-TGA were assigned response of sensitive (IC50 <1 μM), intermediate (IC50 ≥1 and >25% reduction at 10 μM), and resistant otherwise (3D-TGA). FIG. 18A shows dose-response curves for Compound A in a representative subset of susceptible 132 3d-TGA tumor models (lung SW1271, melanoma MEL-JUSO, pancreatic KP-2, colorectal NCI-H747 and thyroid KMH-2) that were sensitive to Compound A and listed in the corresponding Table below the graph in FIG. 18A. FIG. 18B shows dose-response curves for Compound A in a representative subset of intermediate 132 3d-TGA tumor models (melanoma SK-MEL-30, pancreatic PA-TU-8902, liver HEP G2, lung NCi-H2087 and colorectal LS1034) that exhibited an intermediate sensitivity to Compound A and listed in the corresponding Table below the graph in FIG. 18B. FIG. 18C shows dose-response curves for Compound A in a representative subset of resistant 132 3d-TGA tumor models (lung NCI-H1581, gastric NCI-N87, breast HCC-38, ovarian OVCAR-3 and prostate LNCaP) that exhibited an resistance to Compound A and listed in the corresponding Table below the graph in FIG. 18C.

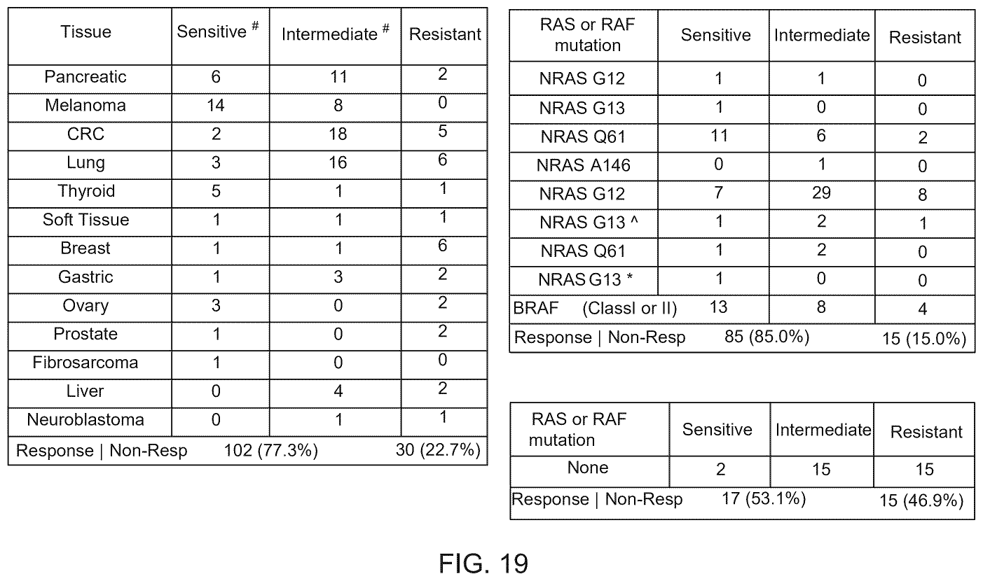

FIG. 19 is a table summarizing the tumor tissue type and RAS mutation status for the humanized 3D tumor growth assay, based on response to Compound A (sensitive, intermediate or resistant).

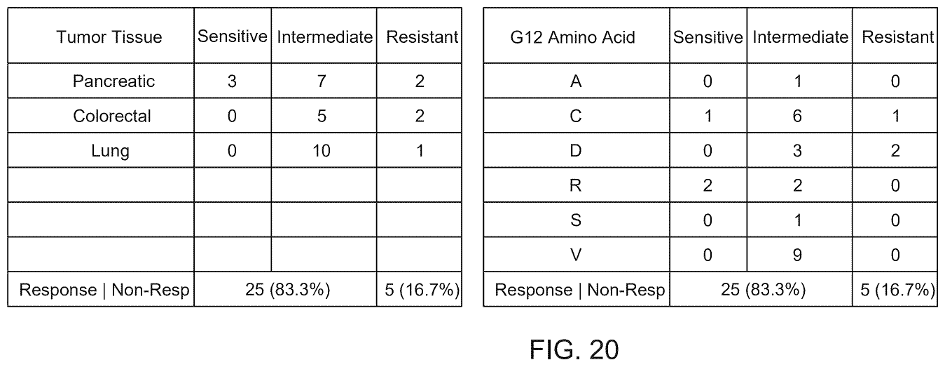

FIG. 20 is a table showing the subset of KRAS G12 variant and associated 3d-TGA response by tumor tissue type and G12 mutation amino acid mutation type.

Compound A was evaluated in 9-point semi-log₁₀ dose response curves from 1 nM to 10 μM across all tumor models in the 3D-TGA. Each model was classified according to the drug dose required to reduce cell division by at least 50% (IC₅₀) as quantified by reduction of tumor cell 5-ethynyl-2′-deoxyuridine (EdU) incorporation. Models were considered sensitive if IC₅₀ <1 μM, intermediate if 1-10 μM or at least 25% inhibition at 10 μM, and resistant otherwise. Each category boundary was established based on attainable in vivo Cmax plasma drug free fractions for Compound A and depth of tumor cell growth impairment.

Tumor models displaying activation mutations in KRAS, NRAS, HRAS, BRAF and certain other MAPK pathway activation events were found to be most sensitive to single agent Compound A. There were 40 putative response biomarkers identified, most of which are known to activate the MAPK pathway upstream of MEK or represent parallel pathways that could confer resistance.

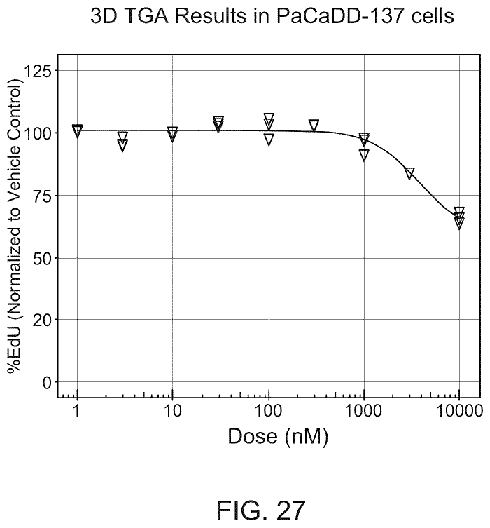

FIG. 27 is a graph showing 3D-TGA Compound A dose responses calculated for PaCaDD-137 cells. The EC50 was calculated as about 3.7 micromolar, and the IC50 was greater than 10 micromolar.

### Example 5

### Compound a as Single Agent and in Combination with Encorafenib for Patients with RAS or RAF Mutant Tumors

Compound A is a novel, oral, dual MEK inhibitor with a unique PK profile, characterized by a high Cmax and a short half-life. Many tumors are addicted to MAPK pathway activation, including the >20% of human tumors with mutations in RAS or RAF. Compound A is an oral once-daily treatment currently in Phase 1 patients with advanced or metastatic RAS-mutant solid tumors [NCT05585320]. To date, drugs disrupting the MAPK pathway have done so chronically, leading to dose-limiting toxicities (DLTs) and poor response durability. In contrast, Compound A was designed to provide deep cyclic inhibition (DCI) of the MAPK pathway via features including a unique pharmacokinetic (PK) profile with high peak plasma drug levels and a near zero, daily drug trough. This promotes pulsatile inhibition of MEK, depriving tumors of sustained signaling of a critical oncogenic pathway while limiting toxicity and durability issues associated with chronic MEK inhibition. In Phase 1a dose escalation, no DLTs were observed, the plasma drug half-life was approximately 2-hours, and pharmacodynamic (PD) data were consistent with DCI. Phase 1b dose expansion is underway.

FIG. 11A is a schematic diagram of an end-to-end translational modeling approach in support of Compound A. FIG. 11B is a table providing certain Phase 1 clinical trial summary parameters for Compound A.

Translational efforts are focused on identifying MAPK pathway addiction and sensitivity to Compound A. Tumor models displaying patient-aligned genomic profiles against large patient databases such as AACR Project GENIE1 were tested in humanized 3D tumor growth assays (3D-TGA). Computational modeling based on response and in-house genomic data was used to inform identification of patient populations for Compound A monotherapy and potential combination opportunities.

Compound A is a short-lived dual MEK inhibitor (MEKi) that works through Deep Cyclic Inhibition (DCI) and is currently under evaluation in early clinical development with an initial focus on advanced RAS mutant solid tumors (NCT05585320). Expanded preclinical and translational modeling support broad potential as monotherapy and in combination for MAPK-pathway mutations upstream of MEK. Assessment of Compound A in over 190 patient-aligned 3D tumor models demonstrated diverse responses across a wide range of MAPK-driven tumor types, including those with RAS or RAF mutations.

Using cancer-specific, patient-aligned cell lines, Compound A activity was characterized in the 3D-TGA. Whole exome sequencing was performed to confirm alteration status, and a further subset subjected to RNA sequencing. Pharmacogenomic data were used to generate a model predictive of response to Compound A and identify biomarker-aligned patient subpopulations. Selected model predictions were then tested in subcutaneous tumor xenograft models in female BALB/c nude mice.

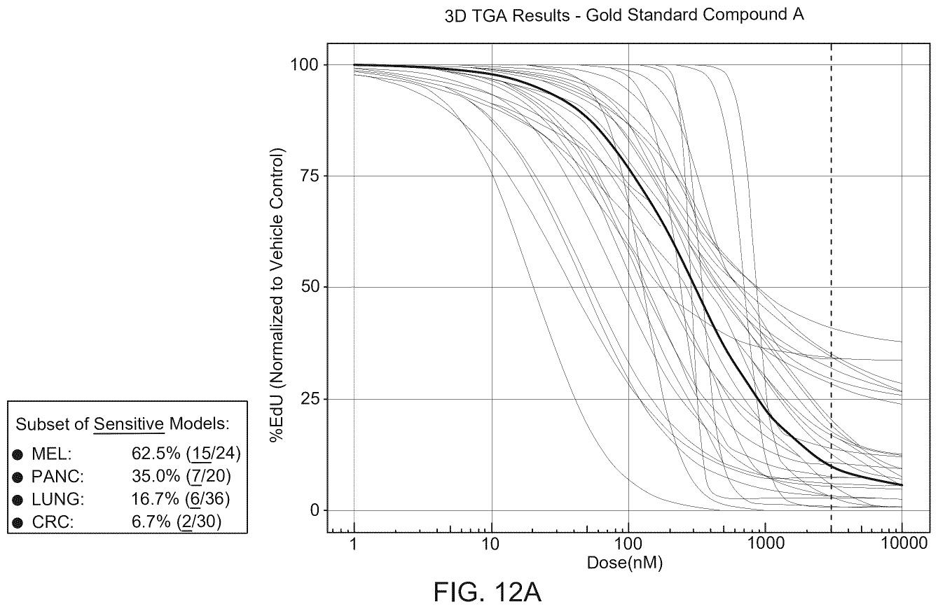

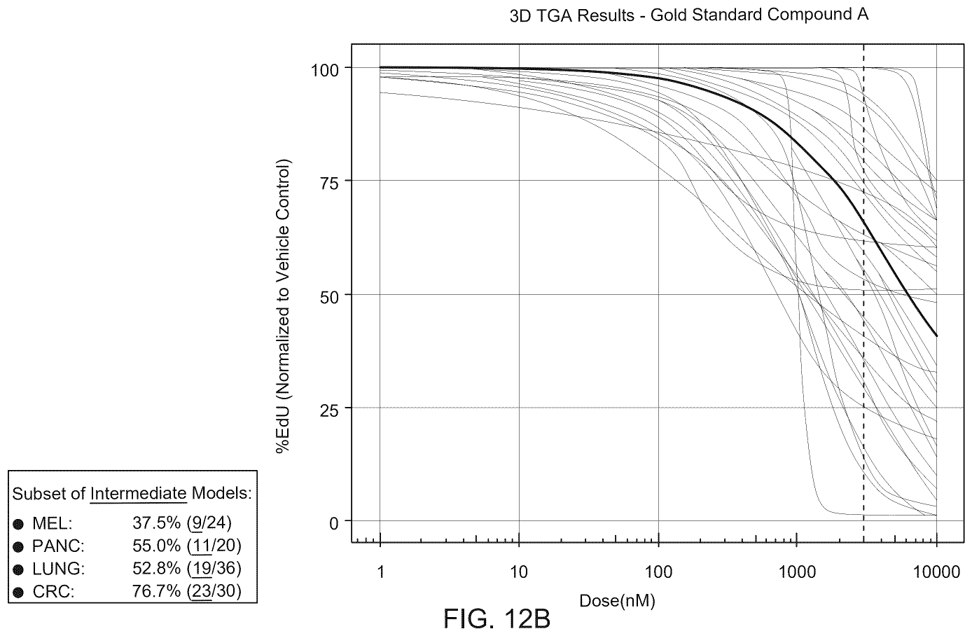

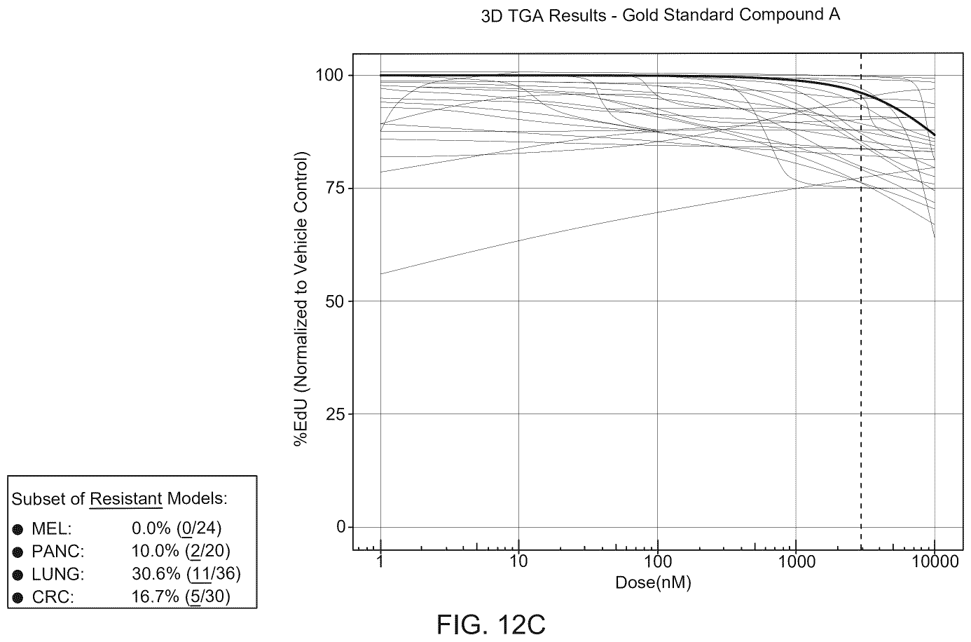

Referring to FIGS. 12A, 12B, and 12C, are graphs obtained from 3D-TGA pharmacogenomics modeling (pharmacology shown). The cell lines tested in 3D-TGA (N=193) were assigned response of sensitive (IC₅₀ <1 uM), intermediate (IC50 ≥1 and >25% reduction at 10 uM), and resistant otherwise. The dark line on each plot represents the median of the individual curves; Dotted vertical line matches Cmax Compound A drug free-fraction levels achieved at 320 mg QD p.o. Major tumor types with activation mutation in the MAPK pathway upstream of MEK (Biomarker Positive): MEL (23/24), PANC (19/20), LUNG (33/36), CRC (28/30). FIG. 12A is a graph showing 3D-TGA Compound A dose responses calculated for sensitive tumor models. FIG. 12B is a graph showing 3D-TGA Compound A dose responses calculated for intermediate tumor models. FIG. 12C is a graph showing 3D-TGA Compound A dose responses calculated for resistant tumor models.

In some embodiments, a therapeutically effective amount of Compound A can be administered for the treatment of a form of melanoma with a RAS or RAF mutation. In some embodiments, a therapeutically effective amount of Compound A can be administered for the treatment of a form of pancreatic cancer with a RAS or RAF mutation. In some embodiments, a therapeutically effective amount of Compound A can be administered for the treatment of a form of lung cancer with a RAS or RAF mutation. In some embodiments, a therapeutically effective amount of Compound A can be administered for the treatment of a form of colorectal cancer with a RAS or RAF mutation.

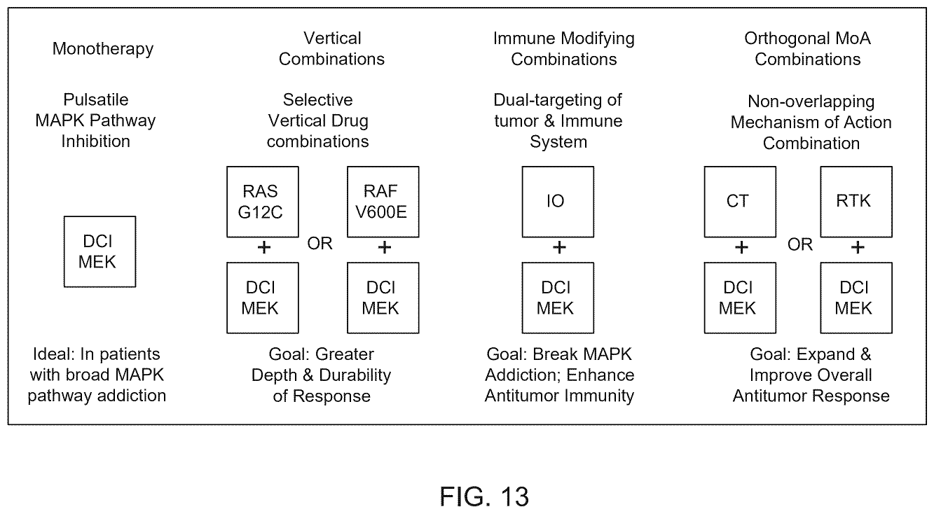

FIG. 13 is a schematic depicting examples of various therapeutic applications of Compound A both as monotherapy and Compound A in combination with other therapies, for the treatment of certain RAS and RAF mutations. Referring to FIG. 13, DCI is an abbreviation for Deep Cyclic Inhibition of the MAP Kinase pathway is a core feature of Immuneering's Dual MEK inhibitor, Compound A, IO is an abbreviation for immune oncology, CT is an abbreviation for Cytotoxic Therapy and RTK is an abbreviation for Receptor Tyrosine Kinase. The deep cyclic inhibition (DCI) of MEK is believed to display strong antitumor activity as monotherapy as well as across broad drug-drug combinations.

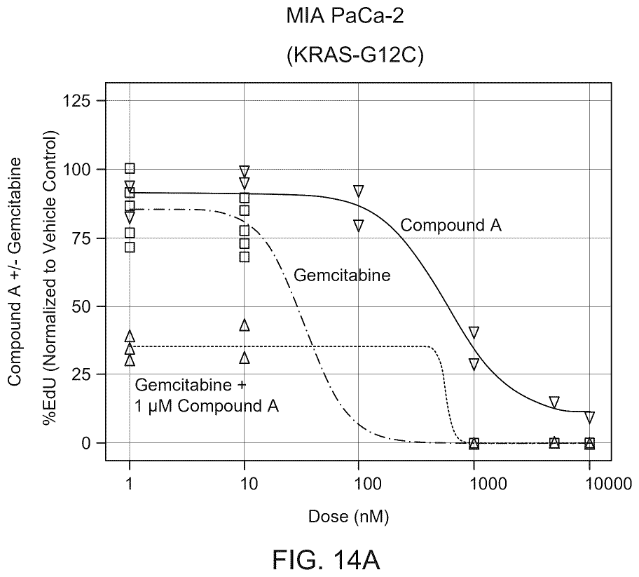

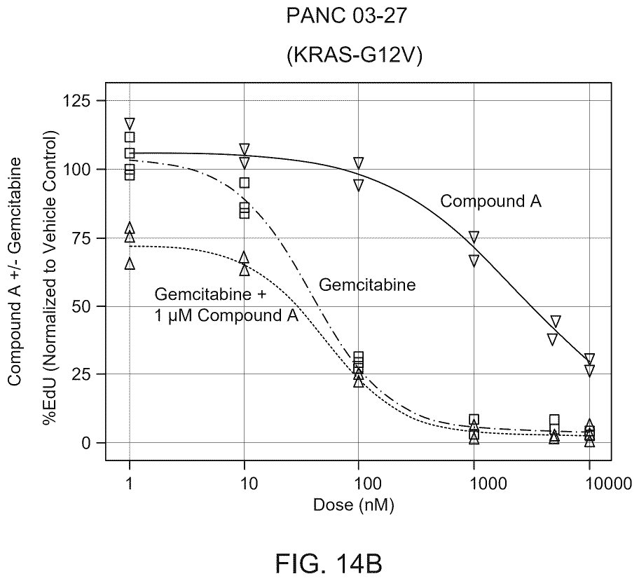

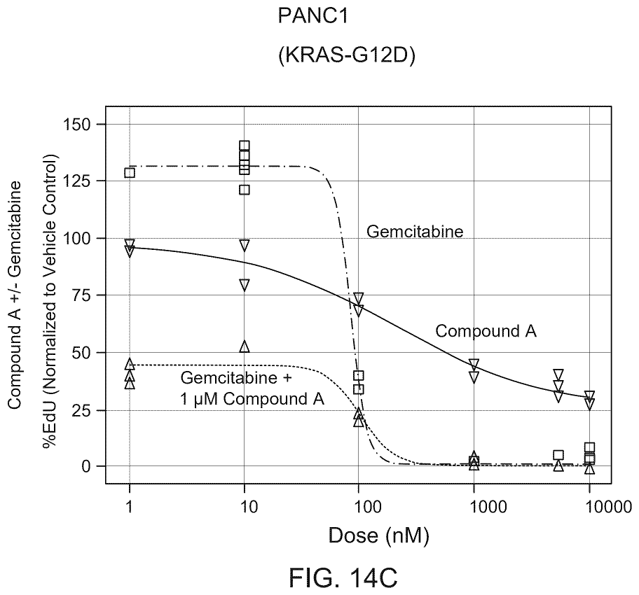

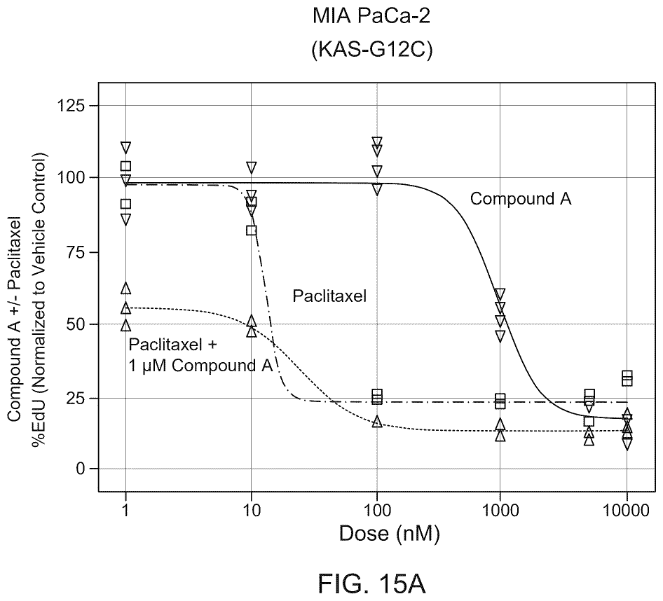

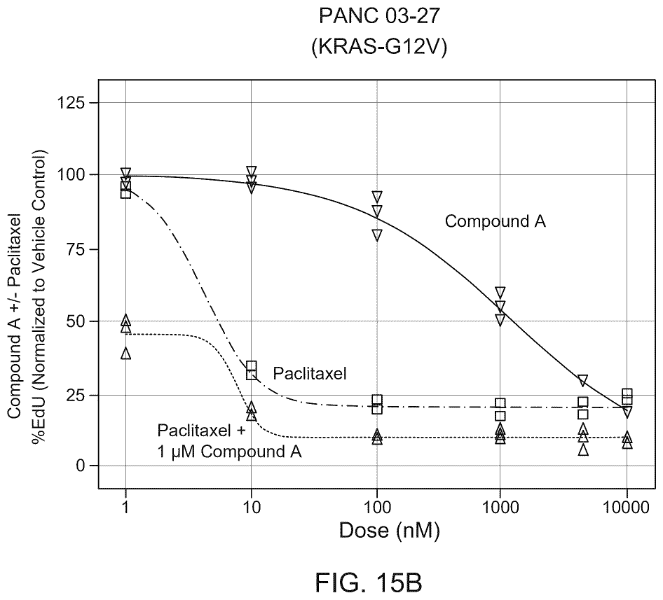

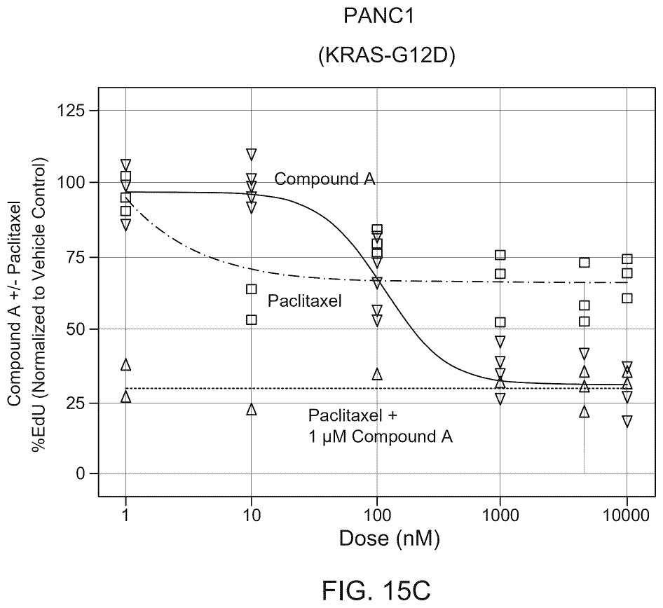

Referring to FIGS. 14A, 14B, 14C, 15A, 15B and 15C: (1) Compound A±CT dose response curves were obtained in the humanized 3D Tumor Growth Assay (3D-TGA); (2) three human pancreatic cancer cell models were selected based on patient alignment scores, where each model's mutational profile mapped to three distinct subsets of GENIE v13.1 patients categorized as pancreatic adenocarcinoma; and (3) gemcitabine and paclitaxel (CT agents commonly used for treatment of pancreatic cancer) were commercially purchased. FIG. 15A is a dose response curves obtained in the humanized 3D Tumor Growth Assay (3D-TGA) with Compound A±paclitaxel with MIA PaCa-2 cells. FIG. 14A is a dose response curves obtained in the humanized 3D Tumor Growth Assay (3D-TGA) with Compound A±gemcitabine with MIA PaCa-2 cells. FIG. 14B is a dose response curves obtained in the humanized 3D Tumor Growth Assay (3D-TGA) with Compound A±gemcitabine with Panc 03-27 (KRAS G12V) cells. FIG. 14C is a dose response curves obtained in the humanized 3D Tumor Growth Assay (3D-TGA) with Compound A±gemcitabine with Panc1 (KRAS-G12D) cells. FIG. 15A is a dose response curves obtained in the humanized 3D Tumor Growth Assay (3D-TGA) with Compound A±paclitaxel with MIA PaCa-2 cells. FIG. 15B is a dose response curves obtained in the humanized 3D Tumor Growth Assay (3D-TGA) with Compound A±paclitaxel with Panc 03-27 (KRAS G12V) cells. FIG. 15C is a dose response curves obtained in the humanized 3D Tumor Growth Assay (3D-TGA) with Compound A±paclitaxel with Panc1 (KRAS-G12D) cells.

An integrated platform of translational experiments and informatics was used to identify patient-aligned model systems, prioritize factors relevant for response to Compound A's unique DCI profile, and elucidate combination opportunities to potentially inform clinical development strategies.

### Example 6

### Activity of Compound a Alone or in Combination with Chemotherapy in RAS-Altered Pancreatic Cancer Models

Addiction to the MAPK pathway drives a large proportion of cancers, and pancreatic tumors almost universally display activation of the MAPK pathway and most commonly by way of RAS mutations. Compound A, a once-daily oral treatment being evaluated in a Phase 1/2a trial for RAS-mutant solid tumors [NCT05585320], offers a novel deep cyclic inhibition (DCI) approach in disrupting the MAPK pathway at MEK. Traditionally, MAPK-targeted drugs inhibit the pathway chronically and are associated with serious class-effect toxicities and limited durability. In contrast, Compound A's unique pharmacokinetic (PK) profile was designed to drive deep pulsatile inhibition of MEK, with the goal of improving tolerability and providing more durable activity across a broad range of MAPK-driven tumors. Phase 1 dose escalation of Compound A revealed no dose-limiting toxicities, high oral bioavailability, plasma half-life of approximately 2-hours, and pharmacodynamic (PD) data supporting DCI of the MAPK pathway.

Compound A responses in humanized 3D tumor growth assays (3D-TGA), which better predict in vivo tumor response relative to 2D culture, were combined with NGS data using machine learning (ML) to refine a pharmacogenomic response model. Evaluation of databases such as AACR Project GENIE enabled prediction of patient alignment of preclinical models based on genomic profile, identification of patient populations displaying MAPK pathway addiction and projected sensitivity to Compound A mono- or combination therapy. To test combinations with approved chemotherapy agents, Compound A, gemcitabine (GEM) and nab-paclitaxel (PAC) and 5-fluorouracil (5-FU) were evaluated in 3D-TGA and in a pancreatic tumor xenograft model with drugs alone or across multiple combinations.

By examining cell line 3D-TGA response data using in-house whole exome sequencing that was further supported by emergent Phase 1 clinical ctDNA data, a machine learning model was further refined to predict Compound A sensitivity. A starting set of gene features, defined by alteration frequency ≥5% in the GENIE 15.0 database within the indications of pancreatic adenocarcinoma, colorectal adenocarcinoma, lung adenocarcinoma, and melanoma, was filtered for representation in the dataset and prioritized using ordinal regression. (GENIE v15.0: The AACR Project GENIE Consortium. AACR Project GENIE: Powering Precision Medicine Through An International Consortium, Cancer Discov. 2017 August; 7(8):818-831). Model-projected responses were then calculated for GENIE patient profiles in select solid tumor indications. Mutation patterns observed in GENIE patients were also compared with those observed in cell lines to identify preclinical models that best resemble real-world patients. The goal of this effort is to increase the translational fidelity of specific tumor models, aiming to translationally identify patient populations most likely to benefit from Compound A treatment. Here, one focus of the model refinement in pancreatic cancer was to better understand MAPK pathway addiction versus utilization.

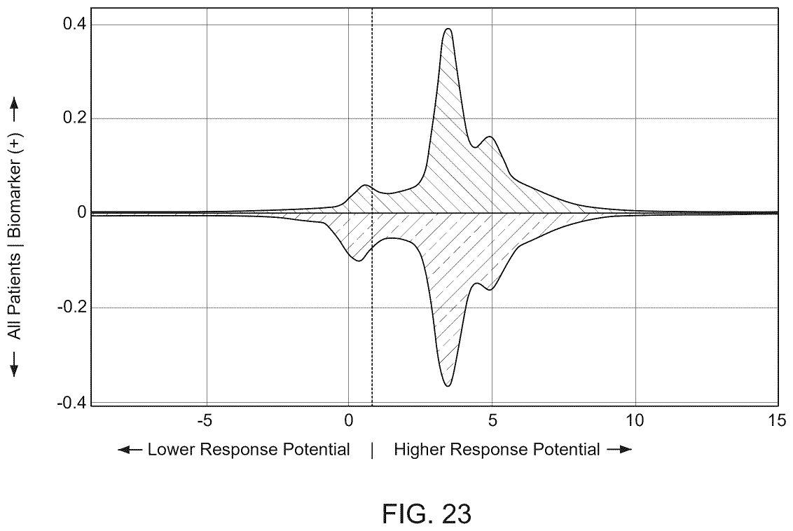

MAPK signaling pathway in PAAD disease and its potential therapeutic sensitivity based on ML model. FIG. 23 is a graph showing the mapping of biomarker sensitive patient populations (GENIE) for pancreatic adenocarcinoma (PAAD). FIG. 23 is a kernel density plot of the probability of MAPK pathway addiction and potentially responsive population projected onto the negative y-axis, and the MAPK biomarker (+) subset projected onto the positive y-axis. Pancreatic cancer displays a strong biomarker (+) subset (91% in this dataset of >4500 pancreatic adenocarcinoma (PAAD) patients in GENIE v15.0 with all model features on the testing panel), suggesting a broad opportunity for mono- and combination therapies with Compound A.

### Example 7

### In Vitro Pharmacology Response Profiles of 12 Pancreatic Tumor Models in Humanized 3D-Tumor Growth Assay (3D-TGA) to Compound A, as Single Agent or Combination Therapy

The humanized 3D tumor growth assay (3D-TGA) is an extracellular matrix (ECM)-based, 3D tumor model that more faithfully mimics the human tumor microenvironment (TME) with the goal of improving translational fidelity to patients. Disruption of the MAPK pathway has specifically demonstrated that 3D models more accurately predict certain aspects of in vivo tumor biology, especially when evaluating molecular activities of drug candidates that target the MAPK pathway. MEK inhibition in the 3D-TGA is useful to compare tumor addiction levels to the MAPK pathway across many models and establish response biomarkers for Compound A. See Casneuf T, et al., “Interleukin-6 is a potential therapeutic target in interleukin-6 dependent, estrogen receptor-α-positive breast cancer,” Breast Cancer (Dove Med Press). 2016; 8:13-27; Onion D, et al. “3-Dimensional Patient-Derived Lung Cancer Assays Reveal Resistance to Standards-of-Care Promoted by Stromal Cells but Sensitivity to Histone Deacetylase Inhibitors,” Mol Cancer Ther. 2016 April; 15(4):753-63; and Sasser A K, et al., “Interleukin-6 is a potent growth factor for ER-alpha-positive human breast cancer,” FASEB J. 2007 November; 21(13):3763-70, and Janes M R, et al., “Targeting KRAS Mutant Cancers with a Covalent G12C-Specific Inhibitor,” Cell. 2018 Jan. 25; 172(3):578-589.e17, each incorporated herein by reference in its entirety.

This Example provides the non-clinical evaluation of various tumor models with different KRAS treated with Compound A, in the 3D-TGA, to evaluate pharmacogenomic responses (sensitivity) as single agent or in combination with chemotherapy agents that are standard of care in the clinic for mPDAC: gemcitabine (an antimetabolite antineoplastic agent interfering with DNA synthesis), paclitaxel (that interferes with the normal function of microtubules during cell division) or 5-FU (that acts in several ways, but principally as a thymidylate synthase (TS) inhibitor and is part of FOLFIRINOX). All drugs were evaluated in 9-point semi-log₁₀ dose response curves from 1 nM to 10 μM across the 12 pancreatic tumor models in the 3D-TGA. For each single drug, an IC₅₀ was calculated (drug dose required to reduce cell division by at least 50% as quantified by reduction of tumor cell 5-ethynyl-2′-deoxyuridine (EdU) incorporation), as well as a synergy score for each combination.

Human tumor cells were maintained in RPMI-1640, supplemented with 10% fetal bovine serum (FBS), 2 mM L-glutamine, 100 U/mL penicillin and 100 ug/mL streptomycin except when additional supplements were recommended by cell banks. Cells were maintained as a 2D monolayer in a humidified incubator at 37° C. with 5% CO₂ and atmospheric air (approximately 21% O₂), being passaged once or twice weekly. All cell lines were validated by IDEXX upon receipt and assay setup. See Appendix for full cell line list.

Cells were seeded in 3D cultures in a black-walled, clear-bottom, non-tissue culture treated, 384-well plate (3000 cells per well) using a 1:1 mixture of BME and a custom media and grown in a humidified incubator with 5% CO₂ and 5% O₂, which approximates capillary levels for each gas, at 37° C. After 24 hours, cells were supplemented with a hormone cocktail made in custom RPMI 1640 media (without phenol red, 1.08 g/L D-Glucose, pH 6.8-6.9) with a final concentration of 250 pg/mL DHT, 50 pg/mL E2, 1 ng/mL P and treated with nine (9) concentrations of reference compounds or Compound A ranging from 10 μM to 1 nM for 72 hours. Cells were assessed for cell proliferation using EdU cell proliferation staining. After 48 hours of treatment, EdU was added to wells to a final concentration of 10 μM. After 72 hours, BME plugs were fixed with 3.7-4.0% formaldehyde 20-30 minutes at room temperature and washed twice with PBS. Cells were permeabilized with 30 μL of filtered 0.5% Triton X-100 in PBS for 20 minutes at room temperature. Samples were washed twice gently with PBS. Cells were stained utilizing click-iT chemistry by adding 20 μL of staining cocktail containing 2 mM copper sulfate, 8 μM sulfo-Cy5-azide, and 20 mg/ml ascorbic acid in PBS and incubated 30-60 minutes at room temperature in dark. Samples were washed three times with PBS, stained with 20 μL DAPI 30-60 minutes at room temperature in dark, washed twice with PBS, 20 μL PBS added to wells, and kept in dark approximately 48 hours before imaging. Plates were imaged with the Molecular Devices Pico ImageXpress. Images were segmented and percentage of cells with EdU incorporation was quantified to calculate antiproliferative effects of the drugs.

Pancreatic tumor models displaying different activating mutations in KRAS were tested in the 3D-TGA to evaluate potential synergy between Compound A, a third generation MEK inhibitor, and chemotherapy agents broadly used in the clinical setting, namely gemcitabine, paclitaxel and 5-fluorouracil. In all cases, addition of Compound A to the chemotherapy did not have any antagonistic effect, and in most cell lines, the presence of the physiologically relevant dose of 1 uM Compound A had an additive effect to all chemotherapy agents tested. Similar combinations with Compound A and chemotherapy agents have also been tested in an in vivo setting with the KRAS^{G12C} mutant model, MIA PaCa2, showing even greater results in terms of synergistic effect.

Five (5)-ethynyl-2′-deoxyuridine (EdU) data was represented as percentage EdU positive cells quantified by high content imaging from the Molecular Devices ImageXpress® Pico. Best fit curves were generated and IC₅₀ calculated using the R package dr4pl (Ritz C, et al., “Dose-Response Analysis Using R.,” PLOS ONE [Internet], 2015 Dec. 30 [cited 2018 Jul. 5]; 10(12):e0146021).

In this study, the pharmacogenomic effect of Compound A was evaluated in a selection of 12 pancreatic tumor models with diverse KRAS mutations in the 3D-TGA, alone and in combination with gemcitabine, paclitaxel or 5-fluorouracil. Drug-drug combinations of Compound A with gemcitabine, paclitaxel, and 5-fluorouracil (5-FU) resulted in enhanced tumor growth inhibition across multiple models when compared to chemotherapy alone. Tables 7A-7E show IC50 values obtained from 3D-TGA with certain chemotherapy agents (paclitaxel in Table 7A, gemcitabine in 7B, 5-FU in Table 7C, oxaliplatin in Table 7D, SN-38 (irinotecan) in Table 7E), and the percent change in IC50 obtained when combining each chemotherapy agent with Compound A in various DAC cancer cell lines. The IC50. A reduction in IC50 when each chemotherapy agent is combined with Compound A is noted as a “YES” in each table. The IC50 values for the chemotherapy agent are as follows: “A”=<50 nM; “B”=50 nM to <100 nM; “C”=100 nM to <500 nM; “D”=500 nM to 1,000 nM; “E”=>1,000 nM.

TABLE 7A
Compound A + Paclitaxel

| | | | | IC50 in 3D-TGA [nM] | | |
|---|---|---|---|---|---|---|
| | | | | | % IC50 change | |
| | PDAC Cell | | | | 1 μM Cmpd A + | IC50 |
| # | Line | Indication | Mutation | Paclitaxel | Paclitaxel | Enhanced |
| 1 | MIA-PaCa2 | PAN | KRAS G12C | A | −32% | YES |
| 2 | Capan-2 | PAN | KRAS G12V | A | −35% | YES |
| 3 | AsPC-1 | PAN | KRAS G12D | A | −92% | YES |
| 4 | PA-TU- | PAN | KRAS G12V | A | −26% | YES |
| | 8988S | | | | | |
| 5 | CFPAC-1 | PAN | KRAS G12V | A | −18% | YES |
| 6 | HPAF-II | PAN | KRAS G12D | A | −(>87%) | YES |
| 7 | Panc 03.27 | PAN | KRAS G12V | A | −(>86%) | YES |
| 8 | PSN-1 | PAN | KRAS G12R | A | 34% | — |
| 9 | Panc 10.05 | PAN | KRAS G12D | A | −18% | YES |
| 10 | Capan-1 | PAN | KRAS G12V | A | −36% | YES |
| 11 | HPAC | PAN | KRAS G12D | A | −43% | YES |
| 12 | PANC-1 | PAN | KRAS G12D | E | −(>100%) | YES |

TABLE 7B
Compound A + Gemcitabine

| | | | | IC50 in 3D-TGA [nM] | | |
|---|---|---|---|---|---|---|
| | | | | | % IC50 change | |
| | PDAC Cell | | | | 1 μM Cmpd A + | IC50 |
| # | Line | Indication | Mutation | Gemcitabine | Gemcitabine | Enhanced |
| 1 | MIA-PaCa2 | PAN | KRAS G12C | A | −(>97%) | YES |
| 2 | Capan-2 | PAN | KRAS G12V | C | +103% | — |
| 3 | AsPC-1 | PAN | KRAS G12D | D | −(>100%) | YES |
| 4 | PA-TU- | PAN | KRAS G12V | A | −10% | YES |
| | 8988S | | | | | |
| 5 | CFPAC-1 | PAN | KRAS G12V | A | −23% | YES |
| 6 | HPAF-II | PAN | KRAS G12D | A | −(>95%) | YES |
| 7 | Panc 03.27 | PAN | KRAS G12V | A | −36% | YES |
| 8 | PSN-1 | PAN | KRAS G12R | A | +26% | — |
| 9 | Panc 10.05 | PAN | KRAS G12D | A | −43% | YES |
| 10 | Capan-1 | PAN | KRAS G12V | A | +34% | — |
| 11 | HPAC | PAN | KRAS G12D | A | +156% | — |
| 12 | PANC-1 | PAN | KRAS G12D | B | −(>99%) | YES |

TABLE 7C
Compound A + 5-Fluorouracil (5-FU)

| | | | | IC50 in 3D-TGA [nM] | | |
|---|---|---|---|---|---|---|
| | | | | | % IC50 change | |
| | PDAC Cell | | | | 1 μM Cmpd A + | IC50 |
| # | Line | Indication | Mutation | 5-FU | Fluorouracil | Enhanced |
| 1 | MIA-PaCa2 | PAN | KRAS G12C | E | — (>90%) | YES |
| 2 | Capan-2 | PAN | KRAS G12V | E | — | — |
| 3 | AsPC-1 | PAN | KRAS G12D | E | — | — |
| 4 | PA-TU-8988S | PAN | KRAS G12V | E | — | — |
| 5 | CFPAC-1 | PAN | KRAS G12V | E | 5% | — |
| 6 | HPAF-II | PAN | KRAS G12D | E | — (>99%) | YES |
| 7 | Panc 03.27 | PAN | KRAS G12V | E | — (>84%) | YES |
| 8 | PSN-1 | PAN | KRAS G12R | E | — | — |
| 9 | Panc 10.05 | PAN | KRAS G12D | E | — | — |
| 10 | Capan-1 | PAN | KRAS G12V | E | — | — |
| 11 | HPAC | PAN | KRAS G12D | E | — | — |
| 12 | PANC-1 | PAN | KRAS G12D | E | — (>90%) | YES |

TABLE 7D
Compound A + Oxaliplatin

| | | | | IC50 in 3D-TGA [nM] | | |
|---|---|---|---|---|---|---|
| | | | | | % IC50 change | |
| | PDAC Cell | | | | 1 μM Cmpd A + | IC50 |
| # | Line | Indication | Mutation | Oxaliplatin | Oxaliplatin | Enhanced |
| 1 | MIA-PaCa2 | PAN | KRAS G12C | E | −62% | YES |
| 2 | Capan-2 | PAN | KRAS G12V | E | −32% | YES |
| 3 | PA-TU-8988T | PAN | KRAS G12V | E | — (>100%) | YES |
| 4 | CFPAC-1 | PAN | KRAS G12V | E | −17% | YES |
| 5 | HPAF-II | PAN | KRAS G12D | E | — (>100%) | YES |
| 6 | PSN-1 | PAN | KRAS G12R | E | — | — |
| 7 | Panc 10.05 | PAN | KRAS G12D | E | −61% | YES |
| 8 | Capan-1 | PAN | KRAS G12V | C | +50% | — |
| 9 | HPAC | PAN | KRAS G12D | E | — | — |
| 10 | PANC-1 | PAN | KRAS G12D | E | — (>100%) | YES |

TABLE 7E
Compound A + SN-38 (Irinotecan)

| | | | | IC50 in 3D-TGA [nM] | | |
|---|---|---|---|---|---|---|
| | | | | | % IC50 change | |
| | PDAC Cell | | | | 1 μM Cmpd A + | IC50 |
| # | Line | Indication | Mutation | SN-38 | SN-38 | Enhanced |
| 1 | MIA-PaCa2 | PAN | KRAS G12C | A | +400% | — |
| 2 | Capan-2 | PAN | KRAS G12V | A | 0% | — |
| 3 | PA-TU- | PAN | KRAS G12V | A | — (>90%) | YES |
| | 8988T | | | | | |
| 4 | CFPAC-1 | PAN | KRAS G12V | A | −75% | YES |
| 5 | HPAF-II | PAN | KRAS G12D | A | — (>89%) | YES |
| 6 | Panc 10.05 | PAN | KRAS G12D | A | −42% | YES |
| 7 | Capan-1 | PAN | KRAS G12V | A | +50% | — |
| 8 | HPAC | PAN | KRAS G12D | A | +269% | — |
| 9 | PANC-1 | PAN | KRAS G12D | A | — (>89%) | YES |

In this study, the pharmacogenomic effect of Compound A was evaluated in a selection of various cancer models with diverse KRAS mutations in the 3D-TGA, alone and in combination with various additional oncology therapeutics.

Tables 8A-8B show IC50 values obtained from 3D-TGA with certain KRAS G12C inhibitor compounds (sotorasib in Table 8A and adagrasib in Table 8B), and the percent change in IC50 obtained when combining each KRAS G12D inhibitor agent with Compound A in various cancer cell lines. A reduction in IC50 when each KRAS G12D inhibitor agent is combined with Compound A is noted as a “YES” in each table. The IC50 values for the agent are as follows: “A”=<50 nM; “B”=50 nM to <100 nM; “C”=100 nM to <500 nM; “D”=500 nM to 1,000 nM; “E”=>1,000 nM.

TABLE 8A
Compound A + Sotorasib

| | | | | IC50 in 3D-TGA [nM] | | |
|---|---|---|---|---|---|---|
| | | | | | % IC50 change | |
| | | | | | 1 μM Cmpd A + | IC50 |
| # | Cell Line | Indication | Mutation | Sotorasib | Sotorasib | Enhanced |
| 1 | MIA-PaCa2 | PAN | KRAS G12C | A | —(>98%) | YES |

TABLE 8B
Compound A + Adagrasib

| | | | | IC50 in 3D-TGA [nM] | | |
|---|---|---|---|---|---|---|
| | | | | | % IC50 change | |
| | | | | | 1 μM Cmpd A + | IC50 |
| # | Cell Line | Indication | Mutation | Adagrasib | Adagrasib | Enhanced |
| 1 | MIA-PaCa2 | PAN | KRAS G12C | A | — (>95%) | YES |

Tables 9A-9B show IC50 values obtained from 3D-TGA with certain CDK-4-6 inhibitor compounds (abemaciclib in Table 9A and palbociclib in Table 9B), and the percent change in IC50 obtained when combining each CDK-4-6 inhibitor agent with Compound A in various cancer cell lines. A reduction in IC50 when each agent is combined with Compound A is noted as a “YES” in each table. The IC50 values for the agent are as follows: “A”=<50 nM; “B”=50 nM to <100 nM; “C”=100 nM to <500 nM; “D”=500 nM to 1,000 nM; “E”=>1,000 nM.

TABLE 9A
Compound A + Abemaciclib

| | | | | IC50 in 3D-TGA [nM] | | |
|---|---|---|---|---|---|---|
| | | | | | % IC50 change | |
| | | | | | 1 μM Cmpd A + | IC50 |
| # | Cell Line | Indication | Mutation | Abemaciclib | Abemaciclib | Enhanced |
| 1 | MIA PaCa-2 | PAN | KRAS G12C | E | — (>100%) | YES |
| 2 | CFPAC-1 | PAN | KRAS G12V | D | −32% | YES |

TABLE 9B
Compound A + Palbociclib

| | | | | IC50 in 3D-TGA [nM] | | |
|---|---|---|---|---|---|---|
| | | | | | % IC50 change | |
| | | | | | 1 μM Cmpd A + | IC50 |
| # | Cell Line | Indication | Mutation | Palbociclib | Palbociclib | Enhanced |
| 1 | MIA PaCa-2 | PAN | KRAS G12C | E | — (>100%) | YES |
| 2 | CFPAC-1 | PAN | KRAS G12V | C | −12% | YES |

Tables 10A-10D show IC50 values obtained from 3D-TGA with certain EGFR inhibitor compounds (afatinib in Table 10A, erlotinib in Table 10B, gefitinib in Table 10C and osimertinib in Table 10D), and the percent change in IC50 obtained when combining each EGFR inhibitor agent with Compound A in various cancer cell lines. A reduction in IC50 when each agent is combined with Compound A is noted as a “YES” in each table. The IC50 values for the EGFR inhibitor agent are as follows: “A”=<50 nM; “B”=50 nM to <100 nM; “C”=100 nM to <500 nM; “D”=500 nM to 1,000 nM; “E”=>1,000 nM.

TABLE 10A
Compound A + Afatinib

| | | | | IC50 in 3D-TGA [nM] | | |
|---|---|---|---|---|---|---|
| | | | | | % IC50 change | |
| | | | | | 1 μM Cmpd A + | IC50 |
| # | Cell Line | Indication | Mutation | Afatinib | Afatinib | Enhanced |
| 1 | MIA | PAN | KRAS G12C | E | —(>100%) | YES |
| | PACA-2 | | | | | |

TABLE 10B
Compound A + Erlotinib

| | | | | IC50 in 3D-TGA [nM] | | |
|---|---|---|---|---|---|---|
| | | | | | % IC50 change | |
| | | | | | 1 μM Cmpd A + | IC50 |
| # | Cell Line | Indication | Mutation | Erlotinib | Erlotinib | Enhanced |
| 1 | MIA | PAN | KRAS G12C | E | — (>100%) | YES |
| | PACA-2 | | | | | |

TABLE 10C
Compound A + Gefitinib

| | | | | IC50 in 3D-TGA [nM] | | |
|---|---|---|---|---|---|---|
| | | | | | % IC50 change | |
| | | | | | 1 μM Cmpd A + | IC50 |
| # | Cell Line | Indication | Mutation | Gefitinib | Gefitinib | Enhanced |
| 1 | MIA | PAN | KRAS G12C | E | — (>100%) | YES |
| | PACA-2 | | | | | |

TABLE 10D
Compound A + Osimertinib

| | | | | IC50 in 3D-TGA [nM] | | |
|---|---|---|---|---|---|---|
| | | | | | % IC50 change | |
| | | | | | 1 μM Cmpd A + | IC50 |
| # | Cell Line | Indication | Mutation | Osimertinib | Osimertinib | Enhanced |
| 1 | MIA | PAN | KRAS G12C | E | — (>100%) | YES |
| | PACA-2 | | | | | |

Table 11 shows IC50 values obtained from 3D-TGA with certain FAK inhibitor compound ifebemetinib and the percent change in IC50 obtained when combining ifebemetinib with Compound A in various cancer cell lines. A reduction in IC50 when ifebemetinib is combined with Compound A is noted as a “YES” in each table. The IC50 values for the EGFR inhibitor agent are as follows: “A”=<50 nM; “B”=50 nM to <100 nM; “C”=100 nM to <500 nM; “D”=500 nM to 1,000 nM; “E”=>1,000 nM.

TABLE 11
Compound A + Ifebemetinib

| | | | | IC50 in 3D-TGA [nM] | | |
|---|---|---|---|---|---|---|
| | | | | | % IC50 change | |
| | | | | | 1 μM Cmpd A + | IC50 |
| # | Cell Line | Indication | Mutation | Ifebemetinib | Ifebemetinib | Enhanced |
| 1 | MIA PaCa-2 | PAN | KRAS G12C | E | — (>100%) | YES |
| 2 | HPAC | PAN | KRAS G12D | E | — | — |
| 3 | HPAF-II | PAN | KRAS G12D | E | — (>100%) | YES |
| 4 | AsPC-1 | PAN | KRAS G12D | E | — | — |
| 5 | Panc 10.05 | PAN | KRAS G12D | E | — (>99%) | YES |
| 6 | PANC-1 | PAN | KRAS G12D | E | — (>100%) | YES |

Table 12 shows IC50 values obtained from 3D-TGA with certain PARP inhibitor compound talazoparib and the percent change in IC50 obtained when combining talazoparib with Compound A in various cancer cell lines. A reduction in IC50 when talazoparib is combined with Compound A is noted as a “YES” in each table. The IC50 values for the EGFR inhibitor agent are as follows: “A”=<50 nM; “B”=50 nM to <100 nM; “C”=100 nM to <500 nM; “D”=500 nM to 1,000 nM; “E”=>1,000 nM.

TABLE 12
Compound A + Talazoparib

| | | | | IC50 in 3D-TGA [nM] | | |
|---|---|---|---|---|---|---|
| | | | | | % IC50 change | |
| | | | | | 1 μM Cmpd A + | IC50 |
| # | Cell Line | Indication | Mutation | Talazoparib | Talazoparib | Enhanced |
| 1 | MIA PACA-2 | PAN | KRAS G12C | E | −93% | YES |
| 2 | Capan-1 | PAN | KRAS G12V | E | — | — |

### Example 14

### Oral Dosage Form of Compound A

An oral dosage form of Compound A can be prepared by combining Compound A as the active pharmaceutical ingredient (API) with pharmaceutically acceptable excipients to form a tablet. The intra granulation excipients can include microcrystalline cellulose (e.g., CEOLUS UF-711), crospovidone Type A (e.g., Polyplasdone XL), hydroxypropyl Cellulose HPC-SL, hydroxypropyl cellulose-Low Substituted L-HPC LH-21, and purified water. The extra granulation excipients can include Compound A and magnesium stearate (e.g., hyqual vegetable source). For example, a tablet dosage form is described in the Table 13 below.

TABLE 13

| | |
|---|---|
| Ingredient | % w/w |

Intra Granulation

| | |
|---|---|
| Compound A (API) | 20.00 |
| Microcrystalline Cellulose | 64.25 |
| Crospovidone, Type A | 5.00 |
| Hydroxypropyl Cellulose HPC-SL | 5.00 |
| Hydroxypropyl Cellulose-Low | 5.00 |
| Substituted, L-HPC LH-21 | |
| Sub Total | 99.25 |

Extra Granulation

| | |
|---|---|
| Compound A (API) granulate | 99.25 |
| Magnesium Stearate | 0.75 |
| Sub Total | 100.00 |

### Example 8

### First-In-Human Clinical Trial of Compound A

Chronic inhibitors of MEK have been poorly tolerated, and limited mainly to RAF mutant disease. Compound A is a deep cyclic inhibitor of MEK in the MAPK pathway. Compound A is a novel, oral, dual MEK inhibitor with a unique PK profile, characterized by a high Cmax and a short half-life. The clinical trial is an open-label study designed to evaluate the safety, tolerability, clinical pharmacology, and preliminary efficacy of Compound A when dosed as monotherapy, once daily, in participants with advanced RAS mutant solid tumors, following at least one line of systemic, standard-of-care therapy.

This is a Phase 1/2a, multicenter, open-label, nonrandomized study to characterize the safety and anti-tumor activity of Compound A administered to participants with RAS-mutated or RAS/MAPK pathway activated advanced or metastatic solid tumors as monotherapy and in combination with approved agents. This study begins with Phase 1 Dose Exploration (including monotherapy dose escalation and dose expansion stages). The monotherapy dose exploration is composed of a dose escalation study followed by the accrual of dose expansion cohorts to generate more robust data on Compound A's safety, tolerability, PK/PD, and antitumor activity.

The dose escalation follows an mTPI-2 design. The mTPI-2 dose escalation scheme in this study allows for a minimum of 1 participant and up to 8 participants at each dose level to be evaluated. The evaluation period encompasses the first 21 days after the first administration of Compound A (i.e., the first 21 days of the first 28-day treatment cycle [Cycle 1]), during which safety and tolerability data for each evaluable participant are collected. These data, along with PK and PD data (if available) and cumulative safety and tolerability data collected, inform dose-escalation decisions within the prespecified planned dose range (40 mg to 960 mg). The initially planned cohort dose escalation levels include: 40, 80, 160, 240, and 320 mg QD.

Once adequate safety, PK, PD, and any efficacy data are available, select tolerable and pharmacologically active dose cohorts are expanded by or enrolled to approximately 20 participants to further evaluate safety, tolerability, PK, PD, and anti-tumor activity. Monotherapy dose escalation participants previously treated at a dose level selected as one of the expansion cohort doses may be counted towards the overall enrollment of that expansion cohort. A preferred dose of Compound A exhibits optimal pharmacological characteristics of deep cyclic inhibition (DCI; high Cmax and near-zero drug trough, approximating a 2 h plasma half-life and >90% inhibition of pERK) and provides adequate anti-tumor activity while not exceeding the MTD (if reached during dose escalation).

Initial phase 1/2a clinical data (NCT05585320) demonstrate favorable initial safety along with PK and PD profiles that are consistent with preclinical modeling of deep cyclic inhibition (DCI), including Cmax levels over 2,000 ng/mL (˜1 μM drug FF), a median plasma t1/2 of 1.94 hours, and ˜90% pharmacodynamic inhibition of pERK.

The study population comprises male and female participants 18 years of age or older with advanced, unresectable, or metastatic RAS-mutated solid tumors who have received at least 1 prior line of systemic therapy for their advanced/metastatic disease. Patients for treatment can have any advanced, unresectable, or metastatic RAS-mutated solid tumors in Phase 1, while Phase 2a will focus on tumor type cohorts: NRAS-mutated melanoma, KRAS-mutated PDAC, KRAS-mutated NSCLC and KRAS-mutated, APC-wildtype CRC.

The cohort dose escalation levels include oral administration of Compound A at doses of 40, 80, 160, 320 mg QD, for up to twelve 28-day cycles. Once daily administration will allow for more than 10 half-lives between doses. Timed serial PK samples are collected on C1D1 and C1D15 and trough PK samples are collected at C1D2, C1D8, C1D16, C2D1 and C3D1.

Table 14 provides clinical data obtained from initial pharmacokinetics (PK) and pharmacodynamics (PD) data from an ongoing phase 1/2a trial (NCT05585320), from certain human subjects enrolled during the first 20 weeks since the first patient was dosed in this Phase 1 clinical trial, showing the calculated half-life based on samples taken at cycle 1 day 1 (C1D1) and cycle 1, day 15 (C1D15), after receiving once-daily oral doses of Compound A at doses of 40 mg, 80 mg, or 160 mg. Each patient was diagnosed with the RAS-mutant cancer diagnosis indicated in Table 14 prior to treatment with Compound A. No dose limiting toxicities (DLT) or severe adverse events (SAE) were observed while dosing these patients through C1D15. No Compound A drug-related adverse events beyond grade 1 were reported in dose levels III or IV. The clinical trial is actively enrolling patients at 320 mg QD p.o. with 2 additional patients already consented (KRAS-G12V pancreatic cancer and KRAS-G12D colorectal cancer). No dose limiting toxicities (DLTs) or serious adverse events (SAEs) were observed in the first 20 weeks since dosing the first patient. Early PK data are approximately dose linear with no Compound A drug accumulation. All patients listed in Table 14 subsequently cleared the dose limiting toxicity (DLT) window.

TABLE 14
Patient Status Summary for Compound A Phase 1 Clinical Trial

| | | RAS | Dose | | C 1 D 1 | C 1 D 15 | Mean | Cmpd A |
|---|---|---|---|---|---|---|---|---|
| # | Patient Cancer | Mutation | Level | Dose | (t_{1/2}) | (t_{1/2}) | (t_{1/2}) | Status |
| 1. | PANCREATIC | KRAS- | I | 40 mg | 1.82 | 2.10 | 1.96 | Off |
| | | G12D | | QD p.o. | hours | hours | hours | Treatment |
| 2. | COLORECTAL | KRAS- | II | 80 mg | 1.41 | 1.43 | 1.42 | Off |
| | | G12V | | QD p.o. | hours | hours | hours | Treatment |
| 3. | COLORECTAL | NRAS- | III | 160 mg | 2.04 | 1.83 | 1.94 | Off |
| | | Q61L | | QD p.o. | hours | hours | hours | Treatment |
| 4. | COLORECTAL | NRAS- | III | 160 mg | 1.91 | 1.97 | 1.94 | On |
| | | Q61K | | QD p.o. | hours | hours | hours | Treatment |
| 5. | PANCREATIC | KRAS- | IV | 320 mg | 2.31 | 2.46 | 2.38 | On |
| | | G12D | | QD p.o. | hours | hours | hours | Treatment |
| 6. | PANCREATIC | KRAS- | IV | 320 mg | 2.04 | 2.27 | 2.16 | On |
| | | G12V | | QD p.o. | hours | hours | hours | Treatment |
| 7. | COLORECTAL | KRAS- | IV | 320 mg | 1.45 | 2.27 | 1.86 | On |
| | | G12S | | QD p.o. | hours | hours | hours | Treatment |

In patients 3 and 4, an approximately 90% pharmacodynamic (PD) inhibition of pERK was observed in patients receiving 160 mg of Compound A administered orally (po) once daily (QD). (Dose Level III, cycle 1, day 1).

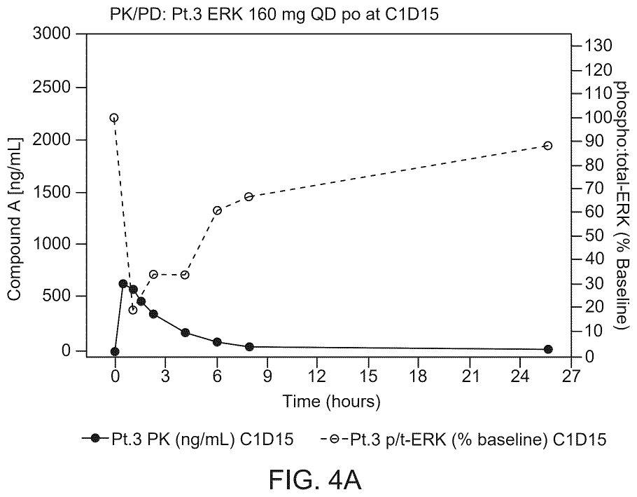

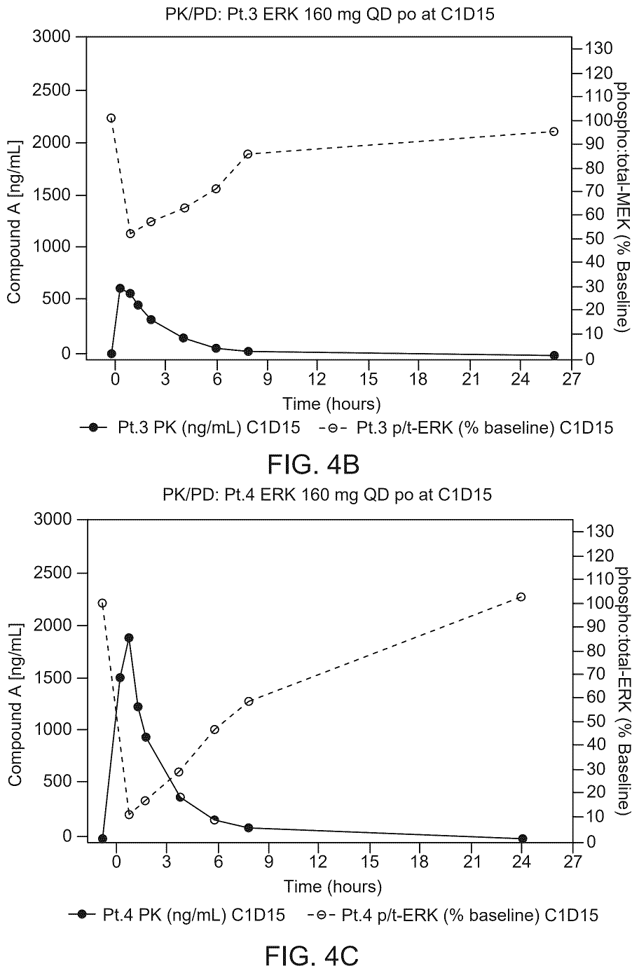

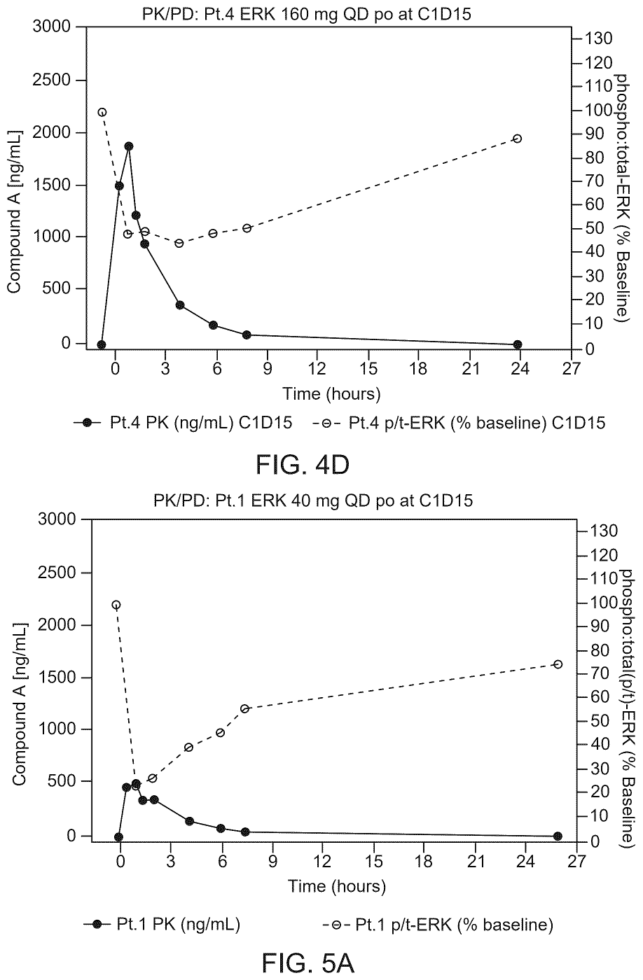

FIG. 4A is a graph showing PK/PD (pERK) for Compound A from patient 3 after receiving 160 mg administered orally (po) once daily (QD) (cycle 1, day 15). FIG. 4B is a graph showing PK/PD (pMEK) for Compound A from patient 3 after receiving 160 mg administered orally (po) once daily (QD) (cycle 1, day 15). FIG. 4C is a graph showing PK/PD (pERK) for Compound A from patient 4 after receiving 160 mg administered orally (po) once daily (QD) (cycle 1, day 15). FIG. 4D is a graph showing PK/PD (pMEK) for Compound A from patient 4 after receiving 160 mg administered orally (po) once daily (QD) (cycle 1, day 15).

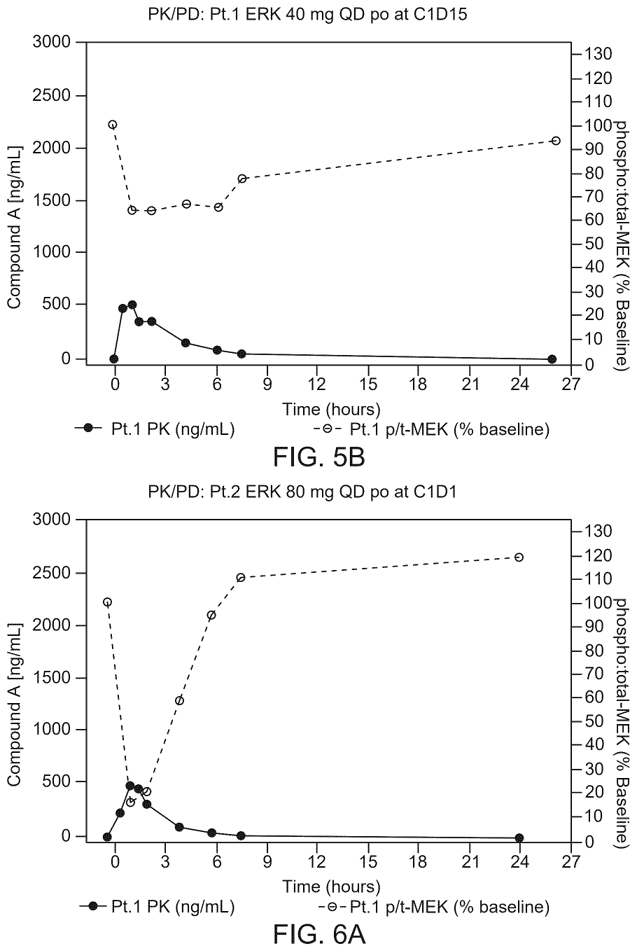

FIG. 5A is a graph showing PK/PD (pERK) for Compound A from patient 1 after receiving 40 mg administered orally (po) once daily (QD) (cycle 1, day 1). FIG. 5B is a graph showing PK/PD (pMEK) for Compound A from patient 1 after receiving 40 mg administered orally (po) once daily (QD) (cycle 1, day 1).

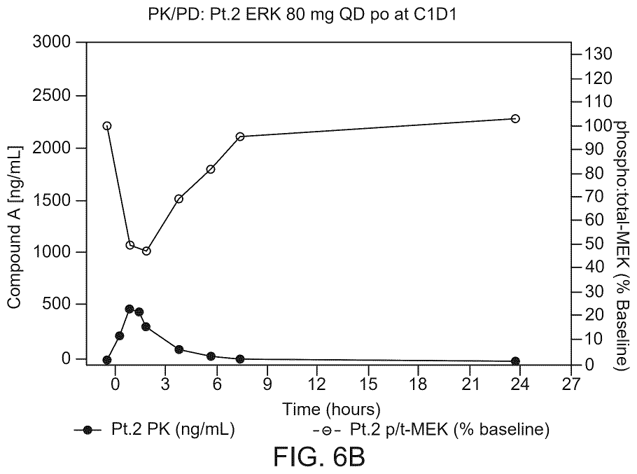

FIG. 6A is a graph showing PK/PD (pERK) for Compound A from patients after receiving 80 mg administered orally (po) once daily (QD) (cycle 1, day 1). FIG. 6B is a graph showing PK/PD (pMEK) for Compound A from patient 2 after receiving 80 mg administered orally (po) once daily (QD) (cycle 1, day 1). Significant PK Cmax levels (plasma concentration of therapy in a specific area of the body) were observed with Compound A of over 2,000 ng/mL (or approximately 1 uM drug free-fraction at 160 mg once daily oral dose). Greater than 90 percent PD inhibition of phosphorylated extracellular signal-regulated kinase (pERK) with Compound A compared to pretreatment baseline for patients at the third dose level (160 mg once daily oral). A median plasma half-life (t_{1/2}) of 1.94 hours observed with Compound A across the first three dose levels evaluable (40 mg, 80 mg and 160 mg once daily oral), in patients with pancreatic and colorectal cancer with different RAS mutations, including KRAS-G12D, the most common mutation presents in pancreatic cancer.

Patient 5 was diagnosed with KRAS-G12D pancreatic cancer and received 320 mg of Compound A once daily, for 15 days in cycle 1. Table 5 provides the corresponding half-life measured at day 1 and day 15. FIG. 7A is a graph showing PK/PD (pERK) for Compound A from patient 5 after receiving 320 mg administered orally (po) once daily (QD) (cycle 1, day 1). FIG. 7B is a graph showing PK/PD (pMEK) for Compound A from patient 5 after receiving 320 mg administered orally (po) once daily (QD) (cycle 1, day 1). FIG. 7C is a graph showing PK/PD (pERK) for Compound A from patient 5 after receiving 320 mg administered orally (po) once daily (QD) (cycle 1, day 15). FIG. 7D is a graph showing PK/PD (pMEK) for Compound A from patient 5 after receiving 320 mg administered orally (po) once daily (QD) (cycle 1, day 15).

Patient 6 was diagnosed with KRAS-G12V pancreatic cancer and received 320 mg of Compound A once daily, for 15 days in cycle 1. Table 5 provides the corresponding half-life measured at day 1 and day 15. FIG. 8A is a graph showing PK/PD (pERK) for Compound A from patient 6 after receiving 320 mg administered orally (po) once daily (QD) (cycle 1, day 1). FIG. 8B is a graph showing PK/PD (pMEK) for Compound A from patient 6 after receiving 320 mg administered orally (po) once daily (QD) (cycle 1, day 1). FIG. 8C is a graph showing PK/PD (pERK) for Compound A from patient 6 after receiving 320 mg administered orally (po) once daily (QD) (cycle 1, day 15). FIG. 8D is a graph showing PK/PD (pMEK) for Compound A from patient 6 after receiving 320 mg administered orally (po) once daily (QD) (cycle 1, day 15).

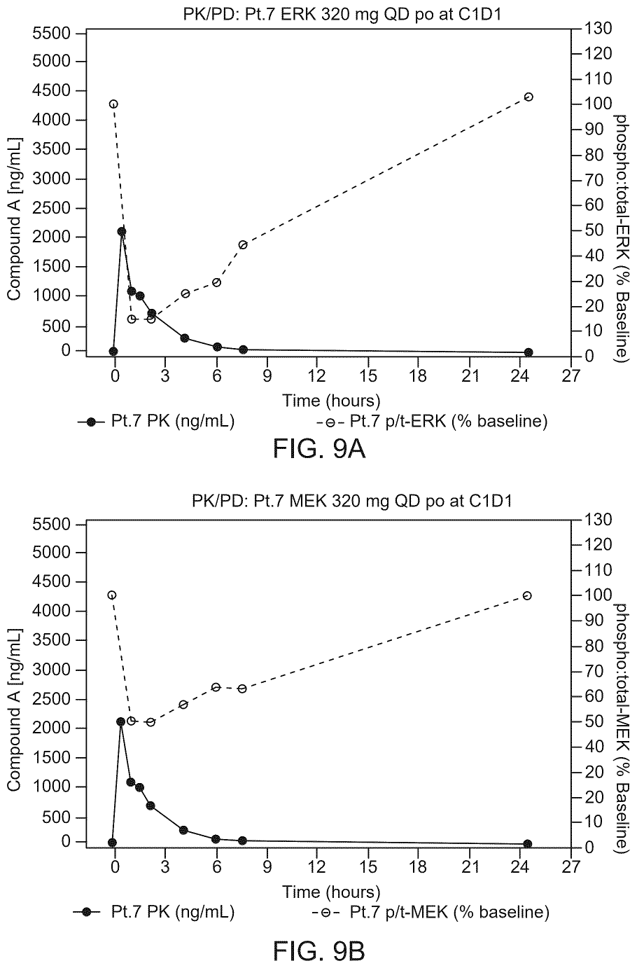

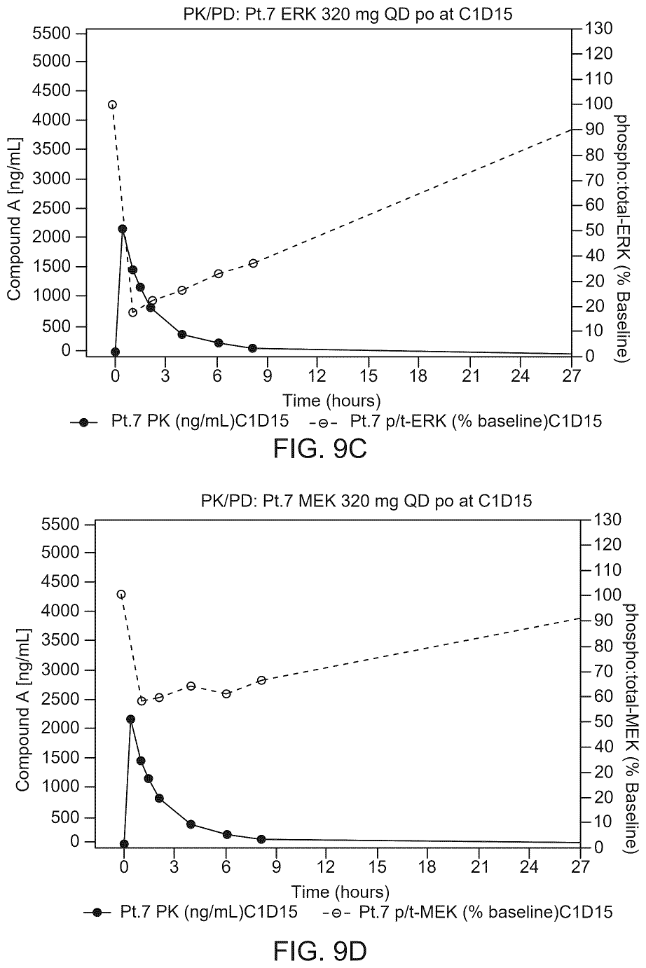

Patient 7 was diagnosed with KRAS-G12S colorectal cancer and received 320 mg of Compound A once daily, for 15 days in cycle 1. Table 5 provides the corresponding half-life measured at day 1 and day 15. FIG. 9A is a graph showing PK/PD (pERK) for Compound A from patient 7 after receiving 320 mg administered orally (po) once daily (QD) (cycle 1, day 1). FIG. 9B is a graph showing PK/PD (pMEK) for Compound A from patient 7 after receiving 320 mg administered orally (po) once daily (QD) (cycle 1, day 1). FIG. 9C is a graph showing PK/PD (pERK) for Compound A from patient 7 after receiving 320 mg administered orally (po) once daily (QD) (cycle 1, day 15). FIG. 9D is a graph showing PK/PD (pMEK) for Compound A from patient 7 after receiving 320 mg administered orally (po) once daily (QD) (cycle 1, day 15). An additional patient was enrolled at 160 mg QD p.o. (NRAS-G13R Melanoma); independent of DLT dose escalation.

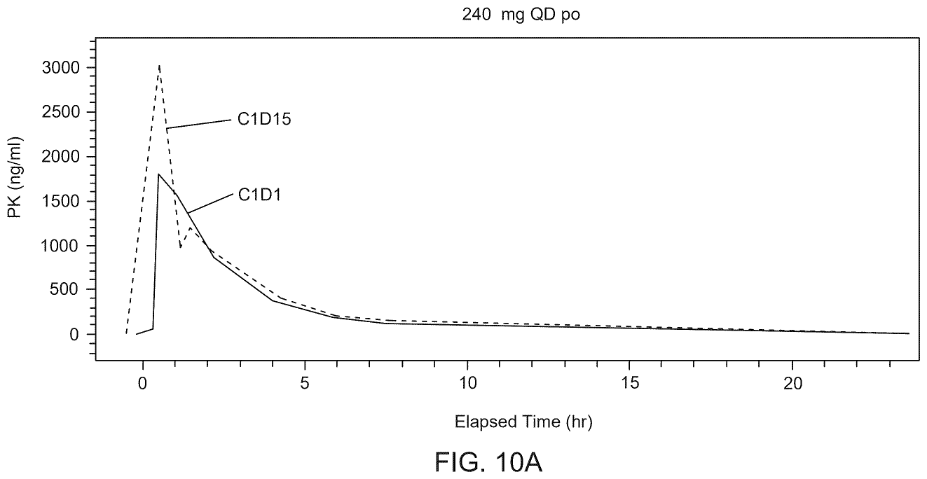

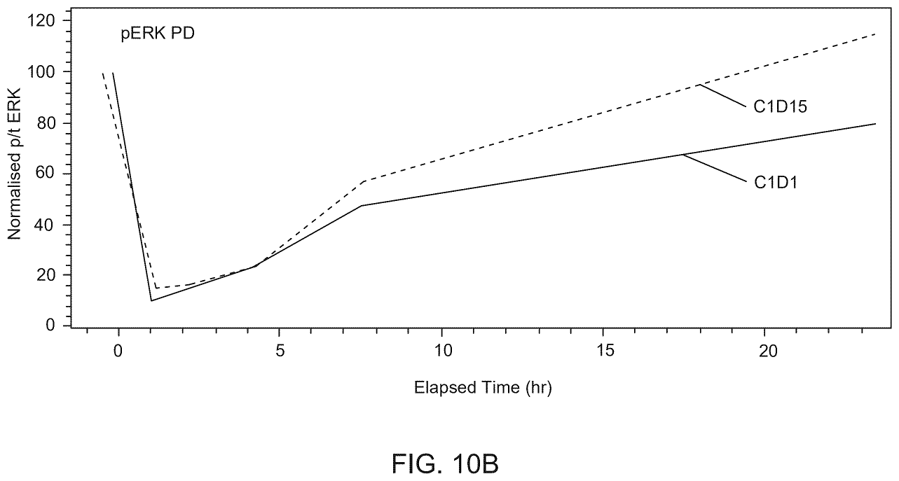

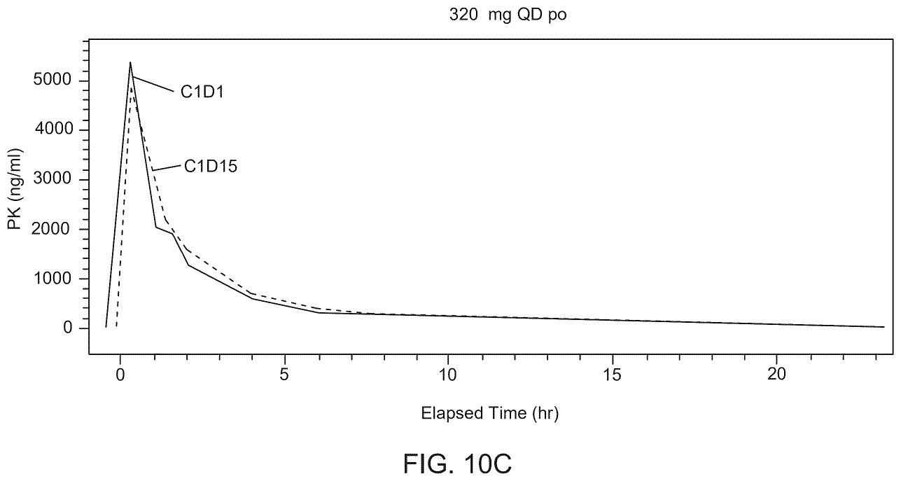

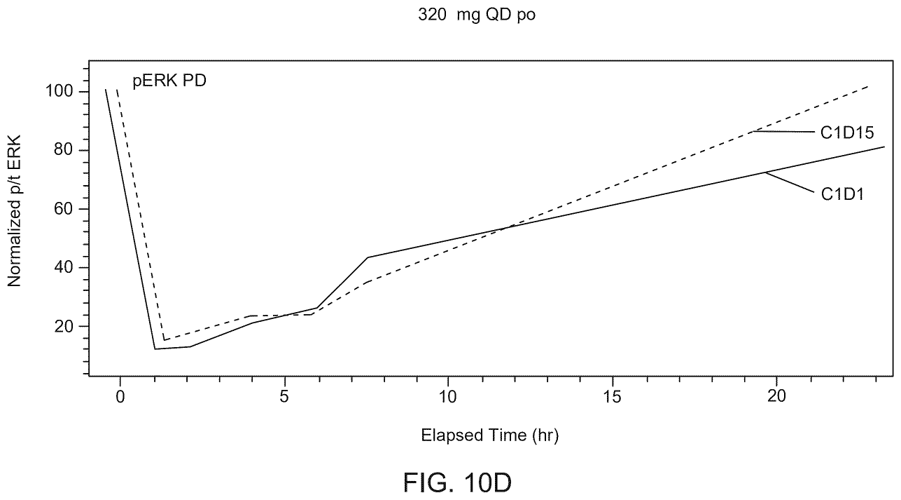

FIG. 10C is a graph showing plasma concentration (PK) for Compound A from patient 16 after receiving 320 mg Compound A administered orally (po) once daily (QD) at cycle 1, day 1 and at cycle 1, day 15. FIG. 10D is a graph showing PK/PD (pERK) for Compound A from patient 16 after receiving 320 mg Compound A administered orally (po) once daily (QD) at cycle 1, day 1 and at cycle 1, day 15.

The cohort dose escalation levels included oral administration of Compound A at a dose of 240 mg QD, for up to twelve 28-day cycles. Once daily administration allowed for more than 10 half-lives between doses. Timed serial PK samples were collected on C1D1 and C1D15 and trough PK samples are collected at C1D2, C1D8, C1D16, C2D1 and C3D1. FIG. 10A is a graph showing plasma concentration (PK) for Compound A from patient 13 after receiving 240 mg Compound A administered orally (po) once daily (QD) at cycle 1, day 1 and at cycle 1, day 15. FIG. 10B is a graph showing PK/PD (pERK) for Compound A from patient 13 after receiving 240 mg Compound A administered orally (po) once daily (QD) at cycle 1, day 1 and at cycle 1, day 15.

This is an example of developing a medicine for a broad population of cancer patients using a universal-RAS therapy in the dose-escalation portion of the Phase 1/2a study for Compound A in patients with advanced solid tumors. The study's Safety Review Committee completed its evaluation and determined that doses up to and including 320 mg (including, for example, 240 mg) once daily were tolerable with no dose limiting toxicities. Enrollment in the Phase 1b expansion portion of the study will commence and is designed to evaluate two dosing cohorts of 12 patients each at an oral dose of 240 mg or 320 mg once daily, with three patients already in the study have so far been dosed at the 320 mg dose level.

This unique therapy aims for Universal-RAS activity: treating patients with any mutation in KRAS, NRAS, or HRAS. As such, there are plans to enroll 21 additional patients in next part of the study to materially add to a dataset for Compound A in patients with RAS-mutant solid tumors.

Compound A achieved significant levels of PK Cmax, demonstrated a half-life of approximately two hours, and was well tolerated with no dose limiting toxicities or serious adverse events observed in patients with pancreatic and colorectal cancer. Upon review of the cumulative data from the eight patients dosed in the Phase 1a study, the SRC endorsed 240 mg daily and 320 mg daily as the dose levels for the expansion cohort and agreed with initiating the Phase 1b expansion.

Compound A is contemplated to achieve universal-RAS activity that selectively impacts cancer cells to a greater extent than healthy cells, through deep cyclic inhibition of the MAPK pathway with once-daily oral dosing. Compound A is currently being evaluated in a Phase 1/2a study in patients with advanced solid tumors harboring RAS mutations for whom there are limited treatment options (NCT05585320).

Compound A was well tolerated with no DLTs or SAEs observed and no drug-related adverse events beyond Grade 1 observed. All patients listed in Table 5 subsequently cleared the dose limiting toxicity (DLT) window. An additional patient was enrolled at 160 mg QD p.o. (NRAS-G13R Melanoma); independent of DLT dose escalation.

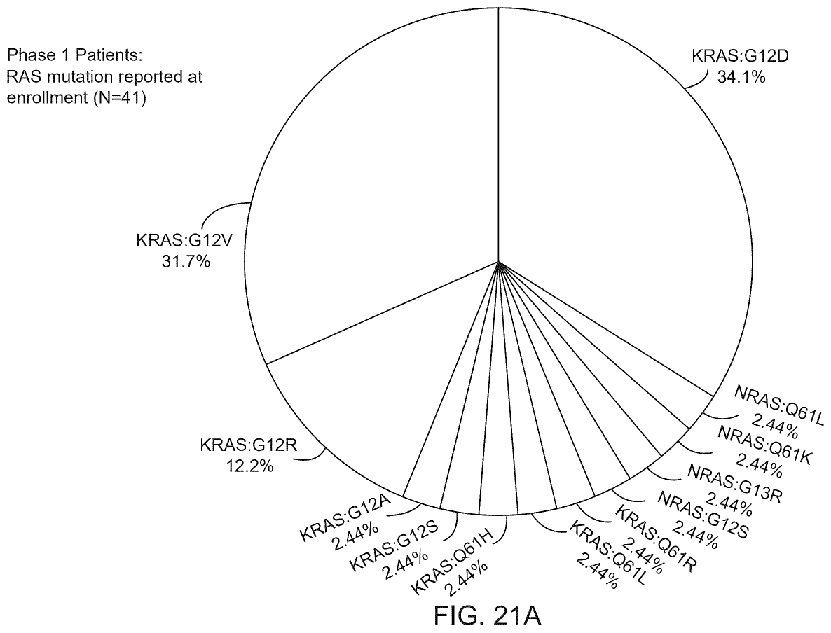

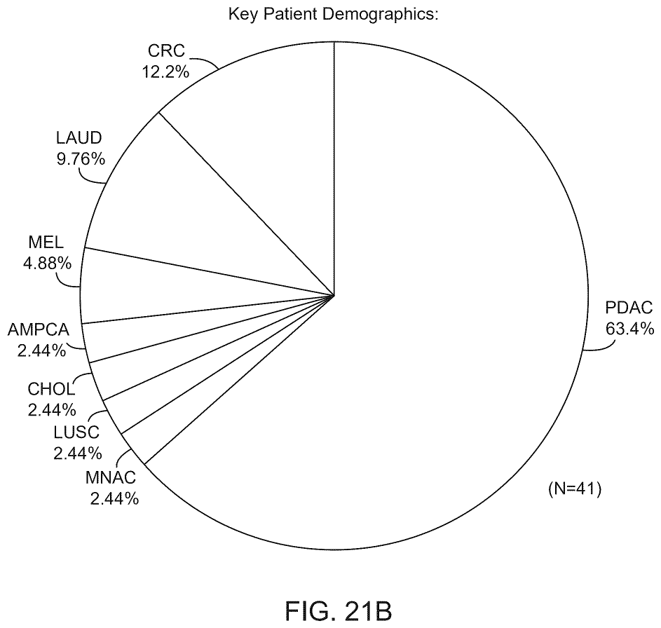

FIG. 21A is a graph showing the RAS mutations reported at enrollment (N=41) for phase 1 human clinical trial patients treated with Compound A. About two-thirds of the patients treated with Compound A as a third or later line of therapy, up to 7th line. RAS mutations were reported in 41 patients diagnosed with various forms of RAS mutated cancer each treated with Compound A (N=41). The 41 patients treated with Compound A included 26 pancreatic ductal adenocarcinoma (PDAC), 5 colorectal cancer (CRC), 4 lung adenocarcinoma (LUAD), 2 melanoma (MEL), 1 lung squamous cell carcinoma (LUSC), 1 ampulla of water carcinoma (AMPCA), 1 mesonephric adenocarcinoma (MNAC), 1 cholangiocarcinoma (CHOL).

FIG. 21B is a graph showing cancer diagnosis for the patients reported at enrollment (N=41) for phase 1 human clinical trial patients treated with Compound A. A total of 82% of the 41 patients treated with Compound A never had a prior partial response (PR) or complete response (CR) to any prior therapy before treatment with Compound A, and the patients had a range of 1-6 prior lines of therapy before receiving Compound A (median 2 prior lines of therapy). The 41 patients treated with Compound A included 34 patients with prior treatment history, of which only 6 are known to have had a PR in response (no CR's) to any prior treatment for metastatic disease (excludes adjuvant). Among these 34 patients with prior treatment history, only 6 are known to have had a partial response (PR) and none are known to have had a complete response (CR), in response to any prior treatment for metastatic disease (excludes adjuvant), Compound A treatment was the median third line of therapy (range 2nd-7th line).

About two-thirds of the patients treated with Compound A as a third or later line of therapy, up to 7^{th} line. Specifically, among 34 patients treated with Compound A, a total of 32.4% received Compound A as a second line therapy, 38.2% of patients received Compound A as a third line therapy, 14.7% received Compound A as a fourth line of therapy, 8.82% received Compound A as a fifth line of therapy, 2.94% received Compound A as a sixth line of therapy and 2.94% received compound A as a seventh line of therapy. RAS mutations were reported in 41 patients diagnosed with various forms of RAS mutated cancer each treated with Compound A (N=41). The 41 patients treated with Compound A included 26 pancreatic ductal adenocarcinoma (PDAC), 5 colorectal cancer (CRC), 4 lung adenocarcinoma (LUAD), 2 melanoma (MEL), 1 lung squamous cell carcinoma (LUSC), 1 ampulla of vater carcinoma (AMPCA), 1 mesonephric adenocarcinoma (MNAC), 1 cholangiocarcinoma (CHOL).

A total of 82% of the 41 patients treated with Compound A never had a prior partial response (PR) or complete response (CR) to any prior therapy before treatment with Compound A, and the patients had a range of 1-6 prior lines of therapy before receiving Compound A (median 2 prior lines of therapy). The 41 patients treated with Compound A included 34 patients with prior treatment history, of which only 6 are known to have had a PR in response (no CR's) to any prior treatment for metastatic disease (excludes adjuvant). Among these 34 patients with prior treatment history, only 6 are known to have had a partial response (PR) and none are known to have had a complete response (CR), in response to any prior treatment for metastatic disease (excludes adjuvant), Compound A treatment was the median third line of therapy (range 2nd-7th line).

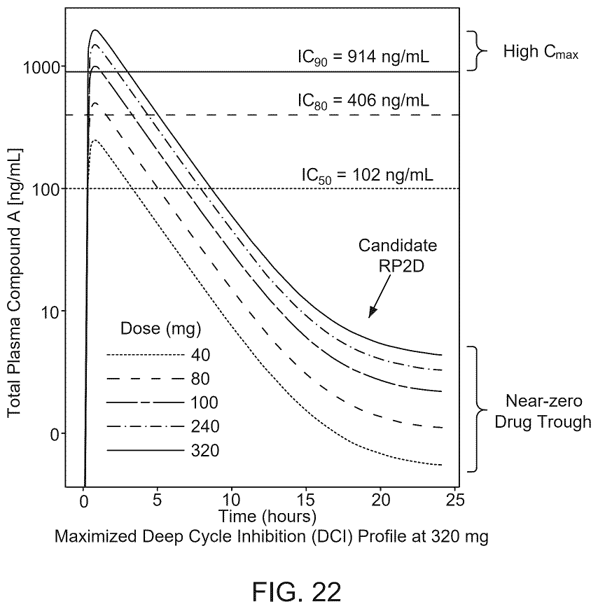

The exploratory PK/PD results of 19 patients treated with Compound A were analyzed (N=19). The Compound A plasma drug concentrations (ng·L) were measured over time after doses of Compound A at 40, 80, 160, 240 and 320 PO QD. FIG. 22 is a graph showing pharmacokinetic (PK) and pharmacodynamic (PD) data obtained from patients treated with Compound A.

Modeled PK profiles were calculated based on measurements of plasma concentrations (ng/mL) of Compound A versus time (h) from 19 patients on a semilogarithmic scale for different dose groups. Approximately dose linear from 40 to 320 mg PO QD with no drug accumulation. A tight relationship was observed between plasma concentrations and phosphorylated ERK (p-ERK) to total ERK (t-ERK) ratios; Longer time above pMEK IC90 at 320 mg (4.0 hr) vs. 240 mg (3.3 hr). Overall, a tight relationship was observed between the plasma concentrations and p-ERK to total ERK ratios, as well as a low variance in PD (p-ERK) profiles at the 320 mg compared to the 240 mg PO QD dose levels. Overall, Compound A was well-tolerated by patients in the clinical trial, with 100% suppression of acquired RAS alterations observed and individual target lesion regression in about half of the patients. Among the patients treated with Compound A:
Best individual lesion regressions: −35.7% at 320 mg in 2 L (vs. −11.4% at 240 mg);
Best RECIST SLD: −18.9% at 320 mg in 2 L (vs. −7.1% at 240 mg);
Longest duration on therapy: 162 days (5+ months) at 240 mg; no TRAEs; and
53% of patients had ≥1 target lesions regress at 320 mg or 240 mg.

A total of 11 patients receiving Compound A had post-baseline scans at 320 mg, 6 patients with post-baseline scans at 240 mg, 3 patients with scans at lower doses, 7 patients have started treatment but not yet scanned, 5 patients are pending data entry and 8 patients progressed before they could receive a post-baseline scan, and 1 was not evaluable. 6 of 20 (30%) patients with completed RECIST scans, showed best sum of longest diameters (SLD) of 0% to −18.9%, and 4 of 20 (20%) showed best SLD less than zero. (Note: 2 patients at 320 mg with −35.7% as best individual lesion regression, both in 2 L).

### Example 9A

### Compound A in Combination with Gemcitabine and Nab-Paclitaxel (mGnP) or in Combination with Modified FOLFIRINOX (mFFX) for Patients with PDAC

In this example, a method of treating pancreatic cancer with Compound A is provided. Specifically, methods of treating pancreatic ductal adenocarcinoma (PDAC) and other exocrine tumors with Compound A are provided.

Table 15 provides a summary of prior clinical trials for the treatment of pancreatic cancer.

TABLE 15
Pancreatic Cancer Clinical Trials

| | | Line of | |
|---|---|---|---|
| Clinical Trial | Treatment | Treatment | ORR |
| Phase III MPACT | Gemcitabine | 1^{st} Line | 7% |
| Phase III MPACT | Gemcitabine + | 1^{st} Line | 23% |
| | nab-paclitaxel | | |
| Phase III | FOLFIRINOX | 1^{st} Line | 32% |
| PRODIGE/ACCORD 11 | | | |
| Phase III NAPOLI-3 | NALIRIFOX | 1^{st} Line | 42% |
| Phase III NAPOLI-1 | Nal-IRI + | 2^{nd} Line | 17% |
| | FU/LV | | |
| Phase III MPACA-3 | mFOLFIRINOX | 2^{nd} Line | 15% |

Compound A is evaluated in a Phase 1/2a, multicenter, open-label, nonrandomized study to characterize the safety and anti-tumor activity of Compound A administered to participants with RAS-mutated or RAS/MAPK pathway activated advanced or metastatic solid tumors as monotherapy and in combination with approved agents. This is a Phase 1/2a, multicenter, open-label, nonrandomized study to characterize the safety and anti-tumor activity of Compound A administered to participants with RAS-mutated or RAS/MAPK pathway activated advanced or metastatic solid tumors as monotherapy and in combination with approved agents. This study is comprised of 2 phases, beginning with Phase 1 Dose Exploration (including monotherapy dose escalation and dose expansion stages, as well as cohorts designed to evaluate the safety and tolerability of Compound A in combination with approved agents) followed by Phase 2a featuring multiple proof-of-concept (POC) generating monotherapy and combination cohorts in malignancies of interest.

Participants are categorized into the following treatment groups (arms):
Treatment Group A: Compound A Monotherapy
Treatment Group B: Compound A in combination with modified gemcitabine and nab-paclitaxel (mGnP) in participants with previously untreated, unresectable advanced or metastatic PDAC
Treatment Group C: Compound A in combination with modified FOLFIRINOX (mFFX) in participants with previously untreated unresectable advanced or metastatic PDAC

Once adequate Compound A monotherapy safety and tolerability data are collected and evaluated, dose escalation cohorts evaluating the safety, tolerability, and PK of Compound A in combination with approved agents is initiated for enrollment in Treatment Groups B and C. In some aspects, the dose of Compound A to be used in combination with the anti-cancer treatments represented in Treatment Groups B and C is 320 mg QD. In addition, Compound A can also be administered in combination with these agents, at 240 mg QD, one dose level lower.

In addition, methods of treatment can include the administration of Compound A in combination with mGnP or mFFX. Approximately 30 participants per indication cohort can be enrolled, based on a cohort-specific Simon's 2-stage design.

Monotherapy Compound A is administered in a once-daily regimen for up to twelve 28-day cycles (48 weeks). If multiple daily dose regimens are pursued (e.g., BID), the total daily dose administered will not exceed the highest tolerable QD dose evaluated to date.

In some methods of treatment, Compound A is administered in combination with approved anti-cancer agents, and Compound A is administered once-daily at the dose that optimally displays DCI, while the combination agents will be administered in commonly used modified regimens that maintain effectiveness while reducing chemotherapy-related toxicities. Combination treatment continues until PD (radiographic or clinical) or other treatment discontinuation criteria are met. Individual components of the chemotherapy regimens can be discontinued if necessary to manage toxicities known to be associated with the agent(s). Likewise, if the entire chemotherapy regimen must be discontinued due to intolerability, participants may continue to receive Compound A as monotherapy until PD or other treatment discontinuation criteria are met. Similarly, participants who achieve a disease response of sufficient depth and duration that may warrant cessation of the approved agents may continue to receive maintenance Compound A until PD or other treatment discontinuation criteria are met. Other recognized maintenance regimens in combination with Compound A may be allowed with approval from the Sponsor MM.

Treatment will be administered in an inpatient or outpatient setting, as deemed appropriate by the Investigator and/or per discussion with the Sponsor. Participant safety will be monitored throughout the study by an SRC established by the Sponsor.

Methods of treatment may include the administration of Compound A in combination with gemcitabine for the treatment of pancreatic adenocarcinoma in a patient in need thereof.

### Example 9B

### Phase 1 Interim Population PK/PD Modeling and Recommended Phase 2 Dose Exploration for Compound A

### Objectives

Activating RAS mutations are present in a third of all cancers. Approved MEK inhibitors chronically inhibit the downstream signaling of RAS, causing significant toxicity. Compound A was designed to have high oral bioavailability and a short half-life with a near-zero drug trough, to achieve Deep Cyclic Inhibition (DCI). Preclinical data indicated that DCI can significantly improve the safety margin by allowing daily pathway recovery in healthy tissues while limiting adaptive resistance in tumor cells. The currently described analysis was designed to identify Phase 2 dosing regimens that achieve the desired DCI pattern in the targeted patient populations.

### Methods

A first-in-human study Compound A recently completed Phase 1 dose expansion in the clinical trial of Example 16A. PK/PD sampling was performed after overnight fasting, pre-dose and at 0.25, 0.5, 1, 1.5, 2, 4, 6, 8 and 24 h post-dose, on Days 1 and 15 of the study (1-24 hours for PD). An ex-vivo surrogate PD endpoint was the inhibition of MEK and downstream ERK phosphorylation in the A549 (KRAS-G12S) cell line, expressed as the ratio of phosphorylated/total kinase, relative to pre-dose baseline. Non-compartmental analysis and population PK/PD analysis were performed using Phoenix® 8.3 and NONMEM® 7.5, respectively.

### Results

Interim PK data are available from 21 out of 45 participants treated with Compound A as single agent at daily doses of 40 mg (n=1), 80 mg (n=1), 160 mg (n=3), 240 mg and 320 mg (n=8 each). Most PK profiles showed a rapid absorption with Tmax within 1 hour of dosing. A two-compartment model with first-order absorption and lag time plus first-order elimination best described the interim PK dataset. Weight-based allometric scaling was applied to clearance, distribution volume and inter-compartmental clearance. The mean elimination half-life at steady-state ranged from 1.4-2.5 hours across doses, without indications of non-linearity. Less than 5% of unchanged Compound A is renally cleared. PD assay results correlated to Compound A plasma concentrations, with maximal inhibition (target engagement) achieved mostly at 1 h, returning to baseline phospho-ERK (pERK) levels between 8 and 24 h. A direct Imax model, with a Hill coefficient fixed to 1, could be successfully applied to describe PK/PD and to calculate time above and below selected inhibitory concentrations. The derived pERK IC50 of 102 ng/mL (48 nM unbound) corresponded well with in vitro pERK inhibition data.

### Example 9C

### Preliminary Phase 1 Safety and Activity of Compound A, an Orally Dosed Universal RAS Inhibitor that Drives Deep Cyclic Inhibition of the MAPK Pathway at MEK, in Patients with Advanced Unresectable or Metastatic Solid Tumors

### Background

About 33% of tumors exhibit activating RAS mutations, making it a significant target for cancer therapy. MEK inhibitors disrupt RAS downstream signaling, but earlier inhibitors have been associated with significant toxicities. Compound A, a MEK inhibitor, inhibits both MEK and ERK phosphorylation, thereby preventing CRAF bypass and hyperactivation of MEK. With a short half-life of 2 hours and once daily dosing (QD), Compound A facilitates deep, pulsatile inhibition of the MAPK pathway at MEK, a process termed Deep Cyclic Inhibition (DCI). This mechanism improves safety and tolerability by allowing daily pathway recovery in healthy tissues while limiting adaptive resistance in tumor cells.

### Methods

A first-in-human Phase 1 study of Compound A was conducted across five U.S. sites. The primary objective was to assess the safety, tolerability and to establish the recommended Phase 2 dose of Compound A in patients with advanced, RAS-mutated solid tumors. A rapid dose escalation scheme was employed with broad inclusion criteria. The study evaluated pharmacokinetics (PK), pharmacodynamics (PD), (circulating tumor DNA) ctDNA, ex vivo pERK and initial clinical efficacy.

### Initial Results

The Phase 1 study enrolled 45 subjects, including 30 with pancreatic ductal adenocarninoma (PDAC), 5 with colorectal cancer, 5 with lung cancer, 2 with melanoma, 1 with cholangiocarcinoma plus 2 other cancers. Two candidate optimal doses, 240 mg and 320 mg QD, were evaluated and supported DCI of the MAPK pathway. Treatment-related adverse events (TRAEs) occurring in ≥10% of patients were transient and limited mainly to grade 1 or 2. No dose-limiting toxicities or serious TRAEs were reported. Early signs of clinical activity in a heavily pretreated, advanced metastatic patient population were promising. Paired CT scans (32) and ctDNA analyses (27) revealed the following: (1.) 21 patients (66%) had a RECIST sum of longest diameters (SLD) <20%, and 8 patients (25%) had an SLD ≤0%; (2.) 13 patients (41%) exhibited regression in at least one target lesion (ranging from −4.8% to −50.0%); (3.) reductions in mean ctDNA levels were observed in 9 patients (33%), with decreases ranging from −24% to −81%; and (4.) no new RAS variants emerged in ctDNA.

### Updated Results

The phase 1 study enrolled 54 subjects, including 34 PDAC, 8 colorectal, 5 lung, 2 melanoma, 2 appendiceal, 1 cholangiocarcinoma plus 2 others. The evaluation of two candidate optimal doses, 240 and 320 mg QD, supported DCI of the MAPK pathway. Treatment-related adverse events (TRAEs) occurring in ≥10% of patients were transient and limited mainly to grade 1 or 2, when observed. No DLTs or serious TRAEs were noted. Although RECIST responses were not observed nor an endpoint of Phase 1, promising signs of clinical activity in heterogeneous, heavily pretreated, advanced metastatic patients were evident. Paired CT scans (40) and ctDNA (36) revealed: (1.) 27 (68%) had SLDs <20%, 9 (23%) had SLDs 0% to −25%, (2.) 15 (38%) had ≥1 target lesion regression (−2.0% to −66.7%), (3.) mean ctDNA reductions were observed in 13 (36%) patients (−3% to −81%), (4.) no new mutation variants in RAS ctDNA and (5.) dose-dependent inhibition ex vivo pERK1. A summary of the total treated patients is described in Table 16.

Compound A safety data suggests that deep cyclic inhibition (DCI) of MEK is well tolerated at doses up to 240 and 320 mg QD po, where Cmax drug free fractions typically exceeded 1,000 nM followed by daily, near-zero drug troughs. DCI was designed to maximally inhibit the MAPK pathway at MED in a pulsatile fashion.

TABLE 16
Total Treated Patients (n = 54) Patients at 240
(N = 19) or 320 mg (N = 30) QD po*

| Maximum Severity | | | | | |
|---|---|---|---|---|---|
| of TRAE's** | | | | | |
| TRAE's occurring | | | | | |
| in >/= 10% of | | | | | Any |
| patients n(%) | Grade 1 | Grade 2 | Grade 3 | Grade 4 | Grade |
| Rash *** | 8(15) | 5(9) | 1(2) | 0(0) | 14(26) |
| Diarrhea | 9(17) | 3(6) | 0(0) | 0(0) | 12(22) |
| Nausea | 8(15) | 1(2) | 0(0) | 0(0) | 9(17) |
| Fatigue | 6(11) | 3(6) | 0(0) | 0(0) | 9(17) |
| Vomiting | 6(11) | 2(4) | 0(0) | 0(0) | 8(15) |
| Subretinal fluid | 7(13) | 0(0) | 0(0) | 0(0) | 7(13) |
| Retinopathy | 3(6) | 3(6) | 0(0) | 0(0) | 6(11) |

*= 54 treated patients also included 1 patient treated at 40 mg QD p0, 1 patient treated at 80 mg po and 3 patients treated at 160 mg QD po.
**= All treatment-related adverse events (TRAE's), regardless of occurrence frequency or grade, were reversible and deemed non-serious ***= preferred terms including in the rash term include dermatitis acneiform, photosensitivity reaction, rash, rash macular, rash maculo-papular, rash pruritic, rash pustular

### Phase 1 Case Study: Patient 1 (KRAS G12V PDAC)

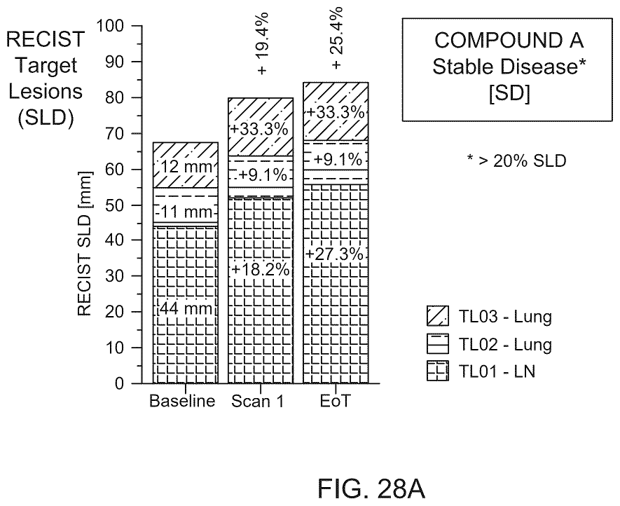

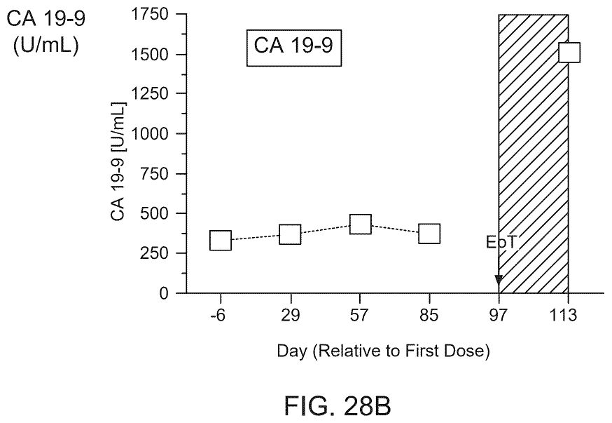

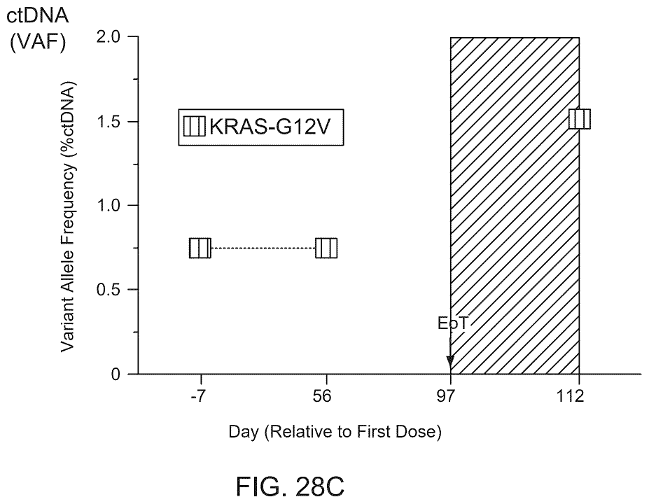

Patient 1 is a 66-year-old Hispanic male diagnosed with metastatic pancreatic cancer (adeno), who received 6 prior lines of treatment (stable disease). The sixth line of treatment was IFL. The patient was then treated with 240 mg QD po of Compound A for 97 days, resulting in stable disease (>20% SLD). FIG. 28A is a bar graph showing RECIST SLD (mm) for target lesions from Patient 1 in a Phase 1 clinical trial treated with Compound A. FIG. 28B is a graph showing CA-19-9 measurements from Patient 1 in a Phase 1 clinical trial treated with Compound A. FIG. 28C is a graph showing ctDNA measurements for KRAS G12V from Patient 1 in a Phase 1 clinical trial treated with Compound A.

### Phase 1 Case Study: Patient 2 (KRAS G12D PDAC)

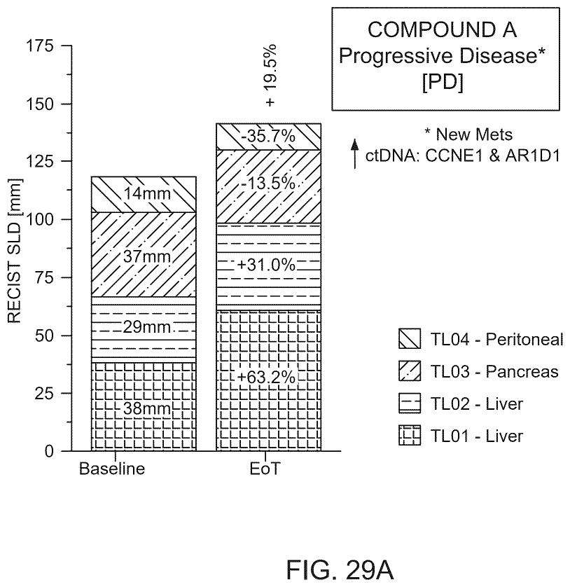

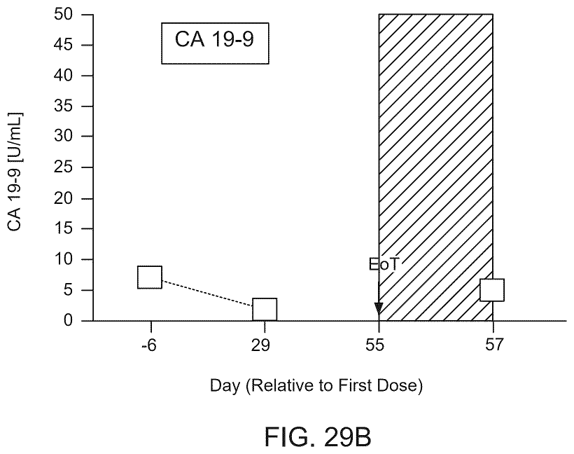

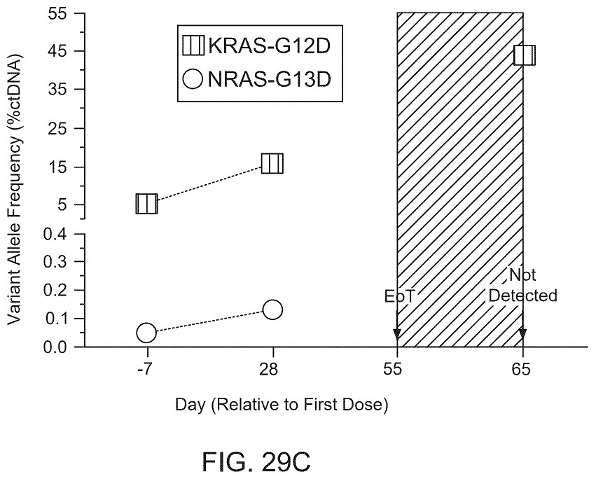

Patient 2 is a 74-year-old Asian male diagnosed with metastatic pancreatic cancer (adeno), who received FOLFIRINOX as a prior line of treatment (partial response). The patient was then treated with 320 mg QD po of Compound A for 55 days. FIG. 29A is a bar graph showing RECIST SLD (mm) for target lesions from Patient 2 in a Phase 1 clinical trial treated with Compound A. FIG. 29B is a graph showing reduction in CA-19-9 measurements from Patient 2 in a Phase 1 clinical trial treated with Compound A. FIG. 29C is a graph showing ctDNA measurements for KRAS G12D and NRAS G13D from Patient 2 in a Phase 1 clinical trial treated with Compound A. The NRAS mutation was identified and eliminated in Patient 2.

### Phase 1 Case Study: Patient 3 (KRAS G12D PDAC)

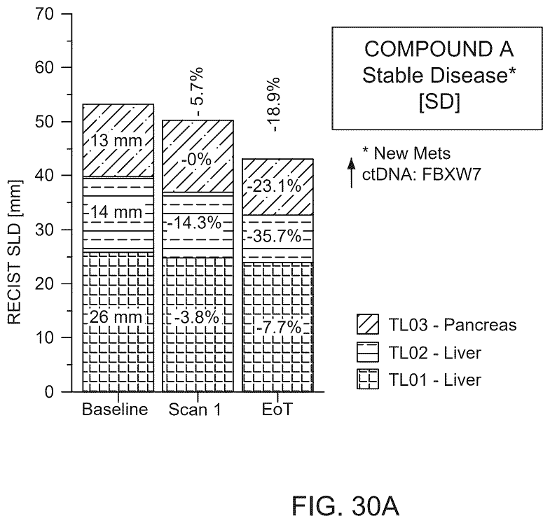

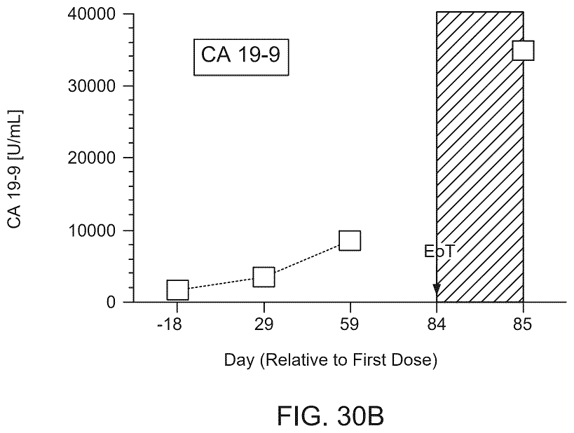

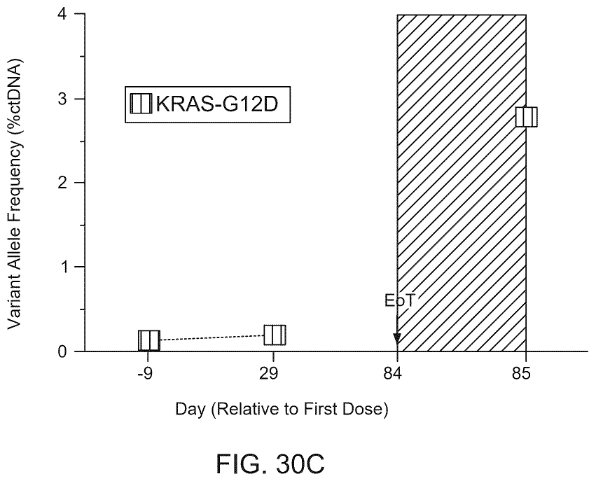

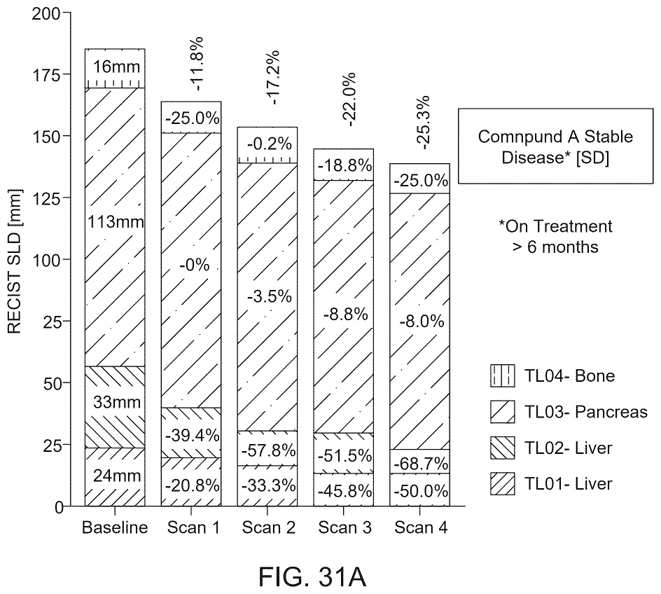

Patient 3 is a 64-year-old Caucasian male diagnosed with metastatic pancreatic cancer (adeno), who received FOLFIRINOX as a prior line of treatment (stable disease). The patient was then treated with 320 mg QD po of Compound A for 84 days, resulting in lesions regressing and 6-8 hours of pain relief. FIG. 30A is a bar graph showing RECIST SLD (mm) for target lesions from Patient 3 in a Phase 1 clinical trial treated with Compound A. FIG. 30B is a graph showing CA-19-9 measurements from Patient 3 in a Phase 1 clinical trial treated with Compound A. FIG. 30C is a graph showing ctDNA measurements for KRAS G12D from Patient 3 in a Phase 1 clinical trial treated with Compound A.

### Phase 1 Case Study: Patient 4 (KRAS G12D PDAC)

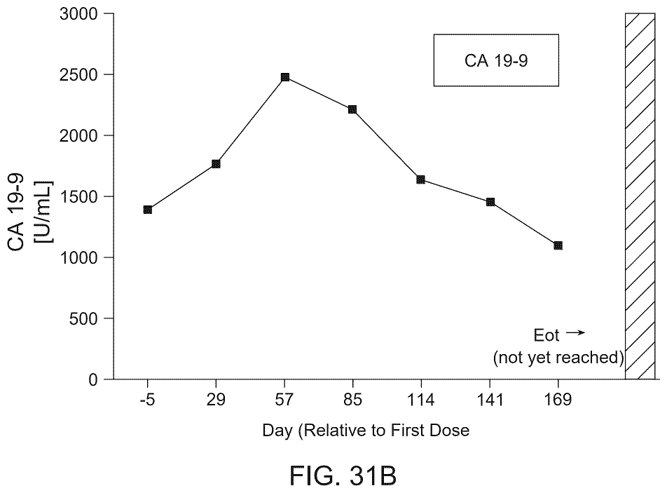

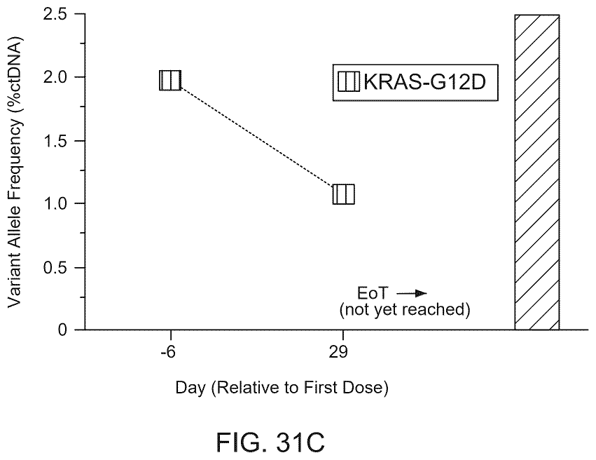

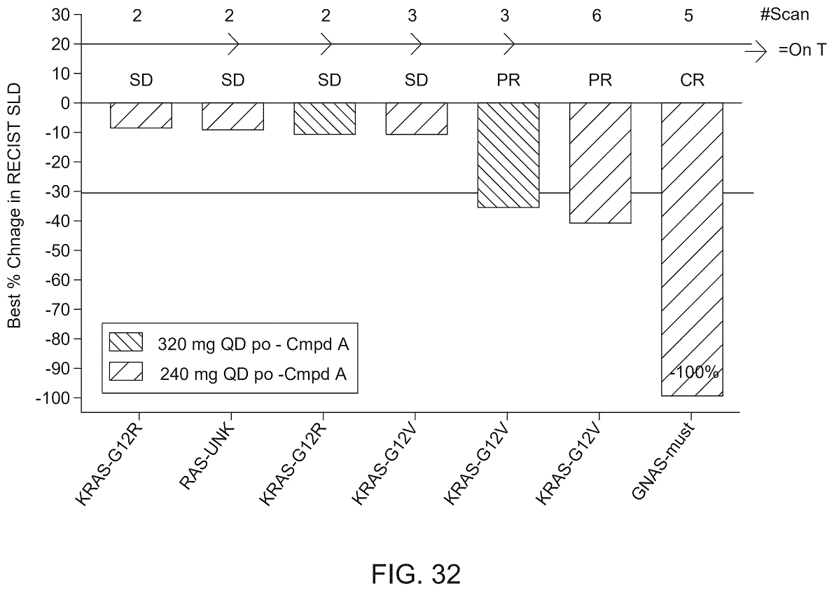

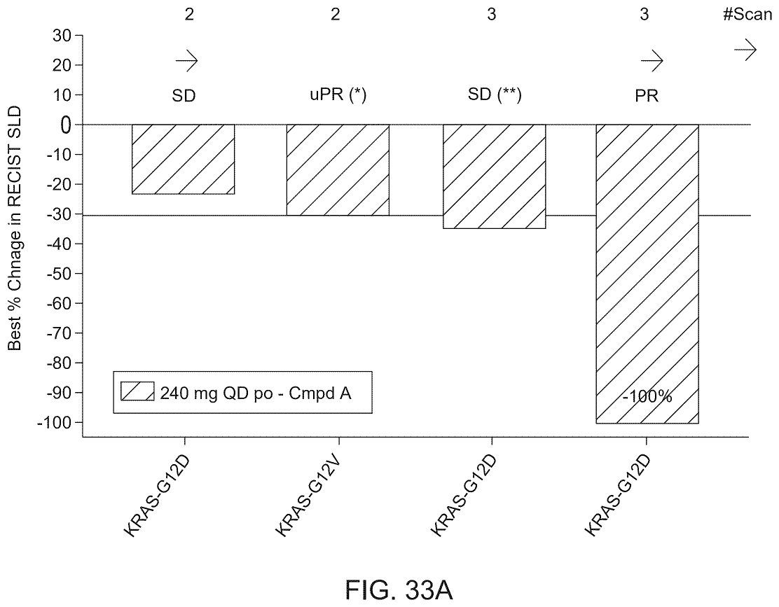

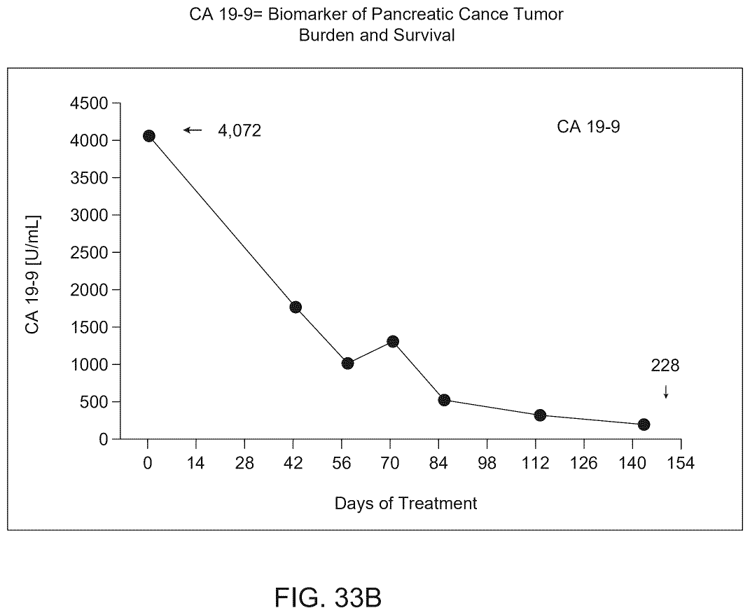

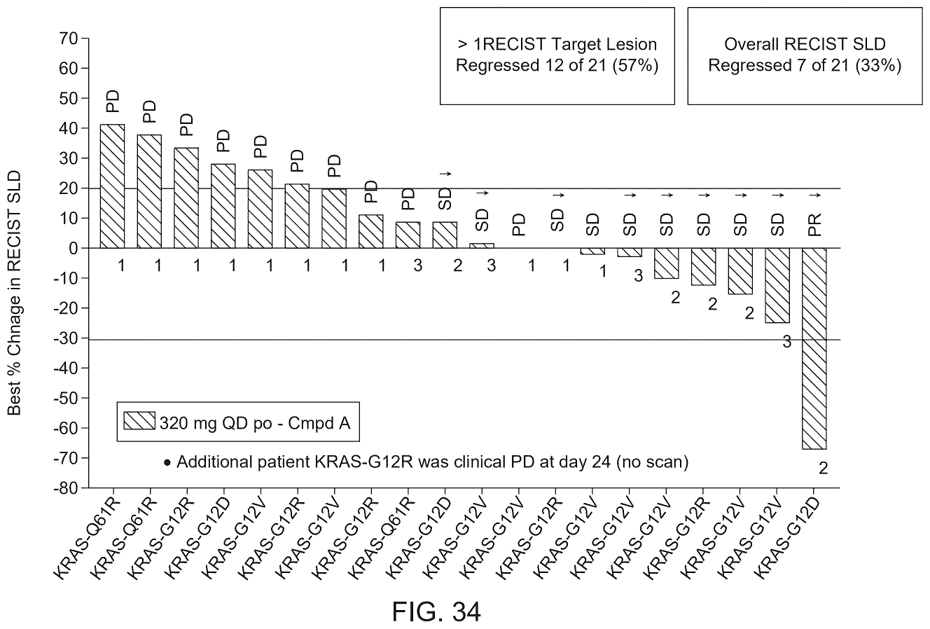

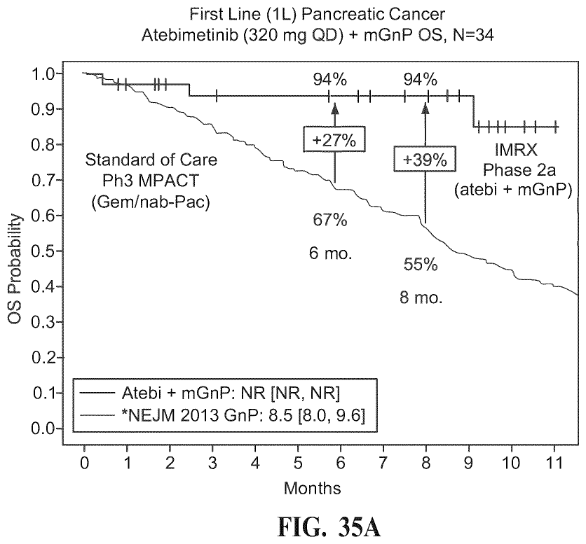

Patient 4 is a 70-year-old Caucasian male diagnosed with metastatic pancreatic cancer (adeno), who received FOLFIRINOX as a first line of treatment (progressive disease) and a combination of gemcitabine/cisplatin/nab-paclitaxel as a second line therapy (progressive disease). The patient was then treated with 240 mg QD po of Compound A for over 150 days, resulting in improved quality of life (QoL) and 7% weight gain, and reduction of CA-19-9 levels and reduction in ctDNA for KRAS G12D. FIG. 31A is a bar graph showing RECIST SLD (mm) for target lesions from Patient 4 in a Phase 1 clinical trial treated with Compound A. FIG. 31B is a graph showing reduction of CA-19-9 measurements from Patient 4 in a Phase 1 clinical trial treated with Compound A. FIG. 31C is a graph showing ctDNA measurements for KRAS G12D from Patient 4 in a Phase 1 clinical trial treated with Compound A. Quality of Life (QoL) was assessed by overall improvement on Functional Assessment of Anorexia/Cachexia Treatment (FAACT) questionnaire (v4) (DOI: 1016670403148).

### Conclusions

Compound A, targeting MAPK-pathway addicted tumors at MEK with through a novel DCI approach, was well tolerated and demonstrated both lesion and molecular level responses as a monotherapy in a heavily pretreated Phase 1 patient population. Phase 2a studies are currently underway.

Compound A is a short-lived dual MED inhibitor (MEKi) that is resistant to CRAF-bypass and has a unique pulsatile pharmacokinetic and pharmacodynamic mechanism (i.e., DCI MEKi). The primary objective of the Phase 1 clinical trial was to evaluate safety, tolerability and to identify a candidate recommended phase 2 dose (RP2D). A candidate RP2D was identified with a highly differentiated safety and tolerability profile. Pharmacodynamic inhibition of pERK upon testing human plasma samples ex vivo exceeded 90% at Cmax for most patients dosed at 240 or 320 mg QD po, providing optimal Compound A DCI MEKi profiles. Antitumor activity was observed at both 240 and 320 mg QD po, as evidenced by deepening RECIST target lesion regressions as well as reductions in CA 19-9 and relevant ctDNA biomarkers. Emerging clinical data suggest that Compound A (i.e., DCI MEKi) provides class-differentiated activity and tolerability that warrants further evaluation in both monotherapy and select combination therapies. Drug efficacy will be further evaluated in a Phase 2a clinical trial (NCT05585320), actively enrolling at 320 mg Compound A QD po monotherapy in melanoma, lung and pancreatic cancer, and at 240 or 320 mg Compound A QD po of Compound A plus gemcitabine- or fluorouracil-containing chemotherapy in first-line pancreatic cancer patients.

### Example 10A

### Preliminary Phase 2a Clinical Trial of Compound A in Pancreatic Cancer

The method of treatment using Compound A is designed to achieve universal-RAS inhibition, selectively targeting cancer cells more than healthy cells through Deep Cyclic Inhibition (DCI) of the MAPK pathway with once-daily dosing. Compound A is currently under investigation in a Phase 1/2a study targeting patients with advanced solid tumors that exhibit RAS mutations, detailed in clinical trial NCT05585320.

In certain formulations, Compound A was administered alongside gemcitabine/nab-paclitaxel for treating pancreatic cancer. Benchmarks established by the Phase 3 MPACT study for first-line treatment with gemcitabine/nab-paclitaxel in pancreatic cancer patients include one complete response (CR) out of 431 patients, a 23% overall response rate, and a 48% disease control rate (Von Hoff et al., N Engl J Med 2013; 369:1691-1703). A modified regimen of gemcitabine/nab-paclitaxel has demonstrated an 18.6% overall response rate (Ahn D H, et al. Therapeutic Advances in Medical Oncology. 2017; 9(2):75-82).

This example, and as otherwise described herein, details findings from the Phase 2a segment of a clinical trial evaluating the efficacy of Compound A in combination with modified gemcitabine/nab-paclitaxel. Initial results showed complete or partial responses in the first two out of five treated patients, reflecting an initial response rate of 40% and an initial disease control rate of 80%. Treatment continues for all five patients. These early outcomes were for dosages of either 240 mg QD or 320 mg QD of Compound A. The combination of Compound A with modified gemcitabine/nab-paclitaxel was well-tolerated, aligning with the existing safety profiles of the individual treatments.

In this example, Compound A was administered to patients diagnosed with pancreatic cancer, such as patients diagnosed with pancreatic ductal adenocarcinoma (PDAC). In general, more than 90% of PDAC patients display mutation in RAS and/or activation of the MAPK pathway. There is a high unmet clinical need in metastatic setting with very limited options for patients. The preclinical and translational data obtained with Compound A support monotherapy and combination opportunities. The Phase 1 Compound A clinical trial sites included many patients with GI-tumor diagnoses (including patients diagnosed with PDAC). A total of 20 patients diagnosed with KRAS mutated PDAC were treated with 320 mg QD of Compound A in a Phase 2 clinical trial. The treated patients had the following KRAS mutations: Q61R (2 patients), G12D (3 patients), G12R (4 patients), and G12V (11 patients). Initial treatments showed stable disease after 1 scan (1 patient), progressive disease after 2 scans (1 patient) or progressive disease after 1 scan (2 patients).

The first two patients in the Phase 2a arm evaluating Compound A with modified gemcitabine/nab-paclitaxel in first line pancreatic cancer have recorded complete or partial responses for an initial response rate of 40% (2/5) and disease control rate of 80% (4/5), with the other three patients earlier in the course of treatment and all five continuing on treatment.

One patient diagnosed with KRAS G12D PDAC having a total SLD of 186 mm (baseline) was treated with 240 mg QD Compound A, resulting in a total SLD reduction of −11.8% in Scan 1, −17.2% SLD reduction in Scan 2, −22.0% SLD reduction in scan 3 and −25% SLD reduction in Scan 4. Prior to treatment with Compound A, this patient received FOLFIRINOX as a first line treatment, and a combination of gemcitabine cisplatin and nab-paclitaxel as a second line therapy, followed by 240 mg QD of Compound A po.

During this study, patients diagnosed with PDAC receive treatment with Compound A as a monotherapy. Compound A or a pharmaceutically acceptable salt thereof is orally administered once daily (QD), at a dose of 320 mg of Compound A. Table 17A is a list of patients who received a combination of Compound A as a monotherapy in a Phase 2a clinical trial.

TABLE 17A

| | | | Baseline | SLD reduction(s) |
|---|---|---|---|---|
| Patient | Mutation | Cmpd A dose | SLD (mm) | (scan no.)** |
| 001 | GNAS | 240 mg QD | 20 | −100%(1) |
| | | | | −100% (2) |
| | | | | −100% (3) |
| 002 | KRAS G12V | 240 mg QD* | 120 | −8.3% (1) |
| | | | | −10% (2) |
| | | | | −40% (3) |
| 003 | KRAS G12V | 240 mg QD* | 74 | −4.1% (1) |
| 004 | KRAS G12R | 240 mg QD* | | |
| 005 | None (WT) | 240 mg QD | 41 | |
| 006 | KRAS G12R | 320 mg QD* | 19 | |
| 007 | KRAS G12D | 320 mg QD* | 101 | |

*prior adjuvant treatment
**the scans were conducted about 6 weeks apart from each other

Table 17B shows initial results from a Phase 2a arm evaluating Compound A in combination with modified gemcitabine/nab-paclitaxel in first line pancreatic cancer patients.

TABLE 17B

| | | Dose | | | | | |
|---|---|---|---|---|---|---|---|
| | | (p.o.) | | | | | |
| | MAPK | Level | % Change | % Change | % Change | % Change | % Change |
| | Mutation | for | in SLD | in SLD | in SLD | in SLD | SLD |
| Patient | Variant | Cmpd A | 1^{st} Scan | 2^{nd} Scan | 3^{rd} Scan | 4^{th} Scan | 5^{th} Scan |
| 1 | GNAS- | 240 mg | −100% | −100% | −100% | −100% | next |
| | T105fsS* | QD | | | | | scan |
| 2 | KRAS- | 240 mg | −8% | −10% | −40% | next | |
| | G12V* | QD | | | | scan | |
| 3 | KRAS- | 240 mg | −4% | next | | | |
| | G12V* | QD | | scan | | | |
| 4 | Unk.^{#} | 240 mg | +6% | next | | | |
| | | QD | | scan | | | |
| 5 | KRAS- | 240 mg | −8.6% | next | | | |
| | G12R* | QD | | scan | | | |

| | |
|---|---|
| Disease Control Rate (DCR): | 80% |

*Detected in prior genetic test
^{#}Unknown (Unk.); MAPK pathway variant not detected in plasma cfDNA or prior genomic test
∘ Partial response result classified as “unconfirmed” pending subsequent scan

During this study, patients diagnosed with PDAC receive treatment with a combination of Compound A and a combination of gemcitabine and nab-paclitaxel. Compound A or a pharmaceutically acceptable salt thereof is orally administered once daily (QD), at a dose of 240 mg of Compound A, or at a dose of 320 mg of Compound A per day. Table 17C is a list of patients who received a combination of Compound A with a combination therapy of gemcitabine/nab-paclitaxel. The phase 2a protocol employs a modified gemcitabine+nab-paclitaxel (mGnP) regimen where the combination therapy of gemcitabine/nab-paclitaxel chemotherapy is administered on Day 1 and Day 15 of each 28-day cycle as follows:
1. Nab-paclitaxel 125 mg/m² via IV infusion over 30 min, followed immediately by,
2. Gemcitabine 1000 mg/m² via IV infusion over 30 min.
3. Then, starting on day 2, Compound A is dosed daily at 240 mg via oral administration.

TABLE 17C

| | | | Baseline | SLD reduction(s) |
|---|---|---|---|---|
| Patient | Mutation | Cmpd A dose | SLD (mm) | (scan no.) |
| 001 | GNAS | 240 mg QD | 20 | −100%(1) |
| | | | | −100% (2) |
| | | | | −100% (3) |
| 002 | KRAS G12V | 240 mg QD* | 120 | −8.3% (1) |
| | | | | −10% (2) |
| | | | | −40% (3) |
| 003 | KRAS G12V | 240 mg QD* | 74 | −4.1% (1) |
| 004 | KRAS G12R | 240 mg QD* | | |
| 005 | None (WT) | 240 mg QD | 41 | |
| 006 | KRAS G12R | 320 mg QD* | 19 | |
| 007 | KRAS G12D | 320 mg QD* | 101 | |

*prior adjuvant treatment

During this study, patients diagnosed with PDAC receive first line treatment with a combination of Compound A and a FOLFIRINOX combination chemotherapy. Compound A or a pharmaceutically acceptable salt thereof is orally administered once daily (QD), at a dose of 240 mg of Compound A, or at a dose of 320 mg of Compound A per day. The patients diagnosed with PDAC receive second line treatment with Compound A as a monotherapy. Compound A was orally administered once daily (QD), at a dose of 240 mg or 160 mg of Compound A per day. Table 17D is a list of patients who received a combination of Compound A with a combination therapy of modified FOLFIRINOX therapy (Patient 010 had a DPD deficiency resulting in inadequate 5FU metabolism, received a reduce dose of 16 mg of Compound A QD and then came off treatment). FOLFIRINOX is a multi-agent chemotherapy regiment composed of the alkylating agent oxaliplatin, the topoisomerase I inhibitor irinotecan, the antimetabolite fluorouracil, and leucovorin, a folic acid analogue given to potentiate the activity of fluorouracil. This protocol employed a modified FOLFIRINOX (mFFX) regimen where the chemotherapy will be administered on Day 1 and Day 15 of each 28-day cycle for up to 6 cycles, as follows:
1. Oxaliplatin 85 mg/m² via IV infusion over 2 h
2. Folinic acid (leucovorin) 400 mg/m² via IV infusion over 2 h
3. Irinotecan 150 mg/m² via IV infusion over 90 min
4. Fluorouracil 2400 mg/m² continuous IV infusion given via home-infusion pump over 46 hours beginning Day 1 and ending on Day 3

TABLE 17D

| | | | Baseline | SLD reduction(s) |
|---|---|---|---|---|
| Patient | Mutation | Cmpd A dose | SLD (mm) | (scan no.) |
| 009 | KRAS G12D | 240 mg QD | 293 | −26.9% (1) |
| | | | | −34.4% (2) |
| | | | | −31.2% (3) |
| 010 | KRAS G12V | 240 mg QD | 200 | −21.5% (1) |
| | | 160 mg QD | | −30.5% (2) |
| 011 | KRAS G12D | 240 mg QD* | 14 | 0 |
| 012 | (unknown) | 240 mg QD | | |
| 013 | KRAS G12D | 240 mg QD | | |

*prior adjuvant treatment

Patients diagnosed with RAS mutated melanoma received treatment with Compound A as a second or third line treatment after treatment with an immuno-oncology therapy. Compound A or a pharmaceutically acceptable salt thereof is orally administered once daily (QD), at a dose of 240 mg of Compound A, or at a dose of 320 mg of Compound A per day.

Patients diagnosed with RAS mutated non-small cell lung cancer received treatment with Compound A as a monotherapy. Compound A or a pharmaceutically acceptable salt thereof is orally administered once daily (QD), at a dose of 240 mg of Compound A, or at a dose of 320 mg of Compound A per day.

Patients diagnosed with RAS mutated appendiceal and cholangiocarcinoma cancer received treatment with Compound A as a monotherapy. Compound A or a pharmaceutically acceptable salt thereof is orally administered once daily (QD), at a dose of 240 mg of Compound A, or at a dose of 320 mg of Compound A per day.

### Example 10B

### Preliminary Phase 2a Clinical Trial of Compound A in Melanoma

In the Phase 2a, Compound A was evaluated as both a monotherapy and in combinations with select, approved chemotherapeutic agents. The Phase 2a portion of the clinical trial included an arm focused on RAS mutant melanoma. In this example, Compound A was administered to patients diagnosed with melanoma, such as patients diagnosed with RAS-mutated melanoma. A total of 5 patients diagnosed with NRAS mutated PDAC were treated with 320 mg QD of Compound A in a Phase 2 clinical trial. The treated patients had the following NRAS mutations: Q61H (1 patient), Q61R (2 patients), G12V (1 patient). Initial treatments showed stable disease after 1 scan (1 patient), or progressive disease after 1 scan (1 patient), with remaining scans pending.

### Example 10C

### Preliminary Phase 2a Clinical Trial of Compound A in Lung Cancer

In the Phase 2a, Compound A was evaluated as both monotherapy and in combinations with select approved chemotherapeutic agents. The Phase 2a portion of the clinical trial included an arm focused on RAS mutant non-small cell lung cancer (NSCLC). In this example, Compound A was administered to patients diagnosed with lung cancer, such as patients diagnosed with RAS-mutated NSCLC. A total of 4 patients diagnosed with KRAS mutated NSCLC were treated with 320 mg QD of Compound A in a Phase 2 clinical trial. The treated patients had the following KRAS mutations: G12D (1 patient), F156L (1 patient), G13A (1 patient) and G12F (1 patient). Initial treatments showed stable disease after 1 scan (3 patients).

### Example 11

### Case Study of Compound A Administered Patient 001 in Phase 2a Clinical Trial Study Described in Example 10A

### Patient Summary:

Patient 001 is a 45-year-old female who was diagnosed with primary adenocarcinoma of the head of the pancreas with metastatic disease to the biliary tract (mPDAC).

### Past Medical/Oncology History:

Past medical history was notable for placement of biliary and hepatic stents due to bile obstruction by the primary tumor, infection due to the stents in right upper quadrant pain, back pain, constipation and vomiting.

### Participation in Compound A Clinical Trial:

The patient consented to enroll in study Compound A Phase 2a—group B safety lead-in with modified Gemcitabine plus nab-Paclitaxel (mGnP) plus Compound A starting on Day 1. The treatment and schedule for modified Gemcitabine plus nab-Paclitaxel (mGnP) in combination with Compound A is detailed below. The first cycle of chemotherapy was administered on 6 Mar. 2024 and the first dose of Compound A was administered on Day 23. On Day 29, all medications had to be held until Day 32 due to Grade 2 elevation of ALT and AST which was caused by the obstruction of the stent. On Day 32, the ALT and AST were grade 1, thus Compound A was restarted at dose of 160 mg daily until Day 36, when the patient went back to full dose of 240 mg daily of Compound A. The patient continued therapy and has not had any other dose interruptions or reductions. The only adverse event the patient experienced related to Compound A was grade 1 abdominal cramping from Day 29 to Day 47.

### Scan Data:

At baseline, the patient was noted to have one target lesion (20 mm lesion in the head of the pancreas) and no non-target lesions were identified. The first on-treatment scan was completed on Day 70 and this showed a complete regression of the target lesion and was an overall complete response per RECIST v1.1. The second on-treatment scan was completed on Day 113 and this confirmed that the lesion was no longer visible and was also determined to be a complete response.

mGnP:

The phase 2a Compound A protocol employs a modified gemcitabine+nab-paclitaxel (mGnP) regimen where chemotherapy is administered on Day 1 and Day 15 of each 28-day cycle as follows:
1. Nab-paclitaxel 125 mg/m² via IV infusion over 30 min, followed immediately by,
2. Gemcitabine 1000 mg/m² via IV infusion over 30 min.
3. Then, starting on day 2, Compound A is dosed daily at 240 mg via oral administration.

### Example 12

### Phase 2a Clinical Trial Data Update for Compound A in Pancreatic Cancer

Administering Compound A in combination with gemcitabine and nab-paclitaxel to patients with pancreatic cancer nearly doubled both the overall response rate (ORR) and the disease control rate (DCR), and provided a 70-fold increase in the complete response (CR) rate in the treated patients. Specifically, a 70-fold increase in the CR was observed in pancreatic cancer patients treated with Compound A in combination with gemcitabine and nab-paclitaxel therapy. Additionally, a surprisingly high (43%) ORR and 86% DCR was observed for patients diagnosed with pancreatic cancer and receiving the combination of Compound A and modified gemcitabine/nab-paclitaxel. These results are nearly double (187% of ORR and 179% of DCR) the corresponding benchmark ORR and DCR rates previously observed for gemcitabine with nab-paclitaxel without Compound A. Furthermore, a highly differentiated tolerability was observed in 96 patients treated with Compound A alone or Compound A in combination with modified gemcitabine/nab-paclitaxel.

The results obtained for Compound A can be compared to prior benchmark results from prior clinical trials without Compound A in Table 18 below.

TABLE 18

| | |
|---|---|
| | Gemcitabine + |
| | nab-Paclitaxel |
| FOLFIRINOX* | (GnP)** |

Efficacy

| | | |
|---|---|---|
| Objective Response Rate (ORR) | 32% | 23% |
| Disease Control Rate (DCR) | 70% | 48% |
| Complete Response (CR) | 0.6% | 0.2% |
| Progression Free Survival (PFS) | 6.4 months | 5.5 months |
| Overall Survival (OS) | 11.1 months | 8.5 months |

Safety

| | | |
|---|---|---|
| Grade 3-4 neutropenia | 46% | 38% |
| Grade 3-4 fatigue | 24% | 17% |
| Grade 3-4 diarrhea | 13% | 6% |

*= Phase III PRODIGE/ACCORD 11 clinical trial
**= Phase III MPACT clinical trial.

The benchmark ORR for pancreatic cancer patients treated with gemcitabine and nab-paclitaxel combination therapy, based on the Phase III MPACT clinical trial, is 23%. The benchmark ORR for treating pancreatic cancer patients treated with modified FOLFIRINOX combination therapy, based on the Phase III PRODIGE/ACCORD 11 clinical trial, is 32%. The benchmark ORR for monotherapy treatments in pancreatic cancer patients, based on Phase II QUILT-3.010 clinical trial, is 3%.

The ORR analysis included all evaluable patients who received their first dose of Compound A at least 14 weeks prior to the cutoff point for the data. This allowed 2 potential scans beyond the baseline scan. Patients who had 2 such scans prior to 14 weeks were also included. Scans typically occur about every 6 weeks (+/−1 week). PD refers to progressive disease. PR refers to partial response. SD reers to stable disease. SLD refers to the sum of the longest tumor diameter. DCR refers to disease control rate (CR+PR+SD). On T refers to On Treatment. Patients treated with 240 mg QD or 320 mg of Compound A combined with the modified GnP therapy demonstrated significantly higher ORR (45%), (DCR (86%), and (14%), compared to the benchmark rates for pancreatic cancer therapies as discussed herein.

In addition, responses were also observed in patients treated with Compound A in combination with modified FOLFIRINOX therapy as both a first line pancreatic cancer treatment (−100% PR) and second line pancreatic cancer treatment (−67% PR). Patients who had 2 such scans prior to 14 weeks were also included. Two patients with subsequently verified rare genomic variants (e.g., DPD, UGT1A1) that significantly impact ability to metabolize chemotherapy had their chemotherapy regimen dose reduced and are excluded in the analysis (both still achieved overall RECIST SLD regressions). The TEAEs observed with Compound A in combination with modified FOLFIRINOX (mFFX) were generally comparable with or favorable to adverse events reported for FFX in Phase III benchmark PRODIGE/ACCORD study.

Overall, the clinical trial results demonstrate methods of treatment comprising the administration of Compound A in combination with the disclosed combination therapies and use of Compound A as a potential replacement of existing MEK inhibitors for patients diagnosed with tumors driven by BRAF, RAS and other conditions.

In addition to combination therapies, Compound A was also evaluated as a monotherapy in pancreatic cancer patients. The ORR observed in these monotherapy treatments was a surprising 167% greater than the benchmark ORR from the Phase II QUILT-3.010 clinical trial. The ORR for this monotherapy treatment was 5%, with 1 one patient achieving a PR, 10 patients achieving SD and 10 patients showing PD. The DCR was 52%. At the time of the data cutoff, 9/21 patients (43%) remained on treatment. The data in

In the event that one of the chemotherapy components must be discontinued due to toxicities, treatment with the remaining agent may be continued in combination with Compound A.

Although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the invention.

What is claimed is:

1. A method of treating pancreatic cancer, comprising:
administering atebimetinib, or a pharmaceutically acceptable salt thereof, to a patient in need thereof once per day (QD) in a total dose providing 240 mg or 320 mg of atebimetinib throughout one or more 28-day treatment cycles, in combination with a modified gemcitabine and nab-paclitaxel chemotherapy (mGnP) chemotherapy, or a modified FOLFIRINOX chemotherapy (mFFX) during the one or more 28-day treatment cycles, to treat the pancreatic cancer in a patient in need thereof, wherein
(a) the mGnP chemotherapy consists of administering to the patient in need thereof only on days 1 and 15 of the 28-day treatment cycle: 125 mg/m² nab-paclitaxel to the patient in need thereof, and administering 1000 mg/m² gemcitabine to the patient in need thereof; and
(b) the mFFX chemotherapy consists of administering to the patient in need thereof only on days 1 and 15 of the 28-day treatment cycle: 85 mg/m² oxaliplatin to the patient in need; administering 150 mg/m² irinotecan to the patient in need thereof; and further administering 2,400 mg/m² fluorouracil to the patient in need thereof beginning on Day 1 and ending on Day 3 of the 28-day treatment cycle.

2. The method of claim 1, wherein the pancreatic cancer is pancreatic ductal adenocarcinoma (PDAC).

3. The method of claim 2, wherein the total dose of atebimetinib is 320 mg, and the atebimetinib is orally administered (po) once daily (QD) to the patient in need thereof throughout the one or more treatment cycles to treat the pancreatic cancer.

4. The method of claim 3, wherein the atebimetinib is administered in combination with mGnP chemotherapy, wherein the method does not comprise administering mFFX chemotherapy for one or more 28-day treatment cycles.

5. The method of claim 4, wherein the pancreatic cancer harbors one or more KRAS mutations.

6. The method of claim 5, wherein the KRAS mutations comprise one or more KRAS G12 mutations.

7. The method of claim 6, wherein the one or more KRAS mutations comprise one or more mutations selected from the group consisting of: G12D, G12V, G12R, G12H and G12C mutations.

8. The method of claim 7, wherein the patient has not previously received treatment for the PDAC.

9. The method of claim 3, wherein the atebimetinib is administered in combination with mFFX chemotherapy, wherein the method does not comprise administering mGnP chemotherapy for one or more treatment cycles.

10. The method of claim 9, wherein the pancreatic cancer harbors one or more KRAS mutations.

11. The method of claim 10, wherein the one or more KRAS mutations comprises a KRAS G12 mutation.

12. The method of claim 11, wherein the patient has not previously received treatment for the PDAC.

13. The method of claim 2, wherein the total dose of atebimetinib is 240 mg, and the atebimetinib is orally administered (po) once daily (QD) to the patient in need thereof throughout the one or more 28-day treatment cycles to treat the pancreatic cancer.

14. The method of claim 13, wherein the pancreatic cancer harbors one or more KRAS mutations.

15. The method of claim 14, wherein the one or more KRAS mutations comprises a KRAS G12 mutation.

16. The method of claim 15, wherein the patient has not previously received treatment for the PDAC.

17. A method of treating pancreatic ductal adenocarcinoma (PDAC) cancer, comprising:
orally administering atebimetinib, or a pharmaceutically acceptable salt thereof, to a patient in need thereof once per day (QD) in a total dose providing 320 mg of atebimetinib, in combination with gemcitabine and nab-paclitaxel to treat the PDAC cancer in the patient in need thereof.

18. The method of claim 17, wherein the PDAC harbors one or more KRAS G12 mutations.

19. The method of claim 18, wherein the one or more KRAS mutations comprises one or more mutations selected from the group consisting of: G12D, G12V, G12R, G12H and G12C mutations.

20. A method of treating pancreatic ductal adenocarcinoma (PDAC) cancer, comprising:
orally administering atebimetinib, or a pharmaceutically acceptable salt thereof, to a patient in need thereof once per day (QD) in a total dose providing 320 mg of atebimetinib, in combination with a chemotherapy comprising oxaliplatin, irinotecan and fluorouracil.
